(19) United States
(12) Patent Application Publication
(10) Pub. No.: US 2023/0390416 A1
(43) Pub. Date: Dec. 7, 2023

(54) COMPOSITIONS FOR INDUCING TUMOR IMMUNITY AND REDUCING DRUG TOLERANCE

(71) Applicant: The Brigham and Women's Hospital, Inc., Boston, MA (US)

(72) Inventors: Aaron Goldman, Melrose, MA (US); Shiladitya Sengupta, Waltham, MA (US); Siva Kumar Natarajan, Ann Arbor, MI (US)

(21) Appl. No.: 18/032,058

(22) PCT Filed: Oct. 18, 2021
(86) PCT No.: PCT/US2021/055434

§ 371 Date: Apr. 14, 2023

Related U.S. Application Data
(60) Provisional application No. 63/092,611, filed on Oct. 16, 2020.

(51) Int. Cl. A61K 47/69; A61K 31/337; A61K 45/06

(52) U.S. Cl. CPC A61K 47/6935; A61K 31/337; A61K 45/06

(57) ABSTRACT

Described herein are HSP-90 inhibitors conjugated to lipids, compositions comprising the conjugates, and methods of use thereof for treating cancer.

### CLAIM OF PRIORITY

This application claims the benefit of U.S. Provisional Application Ser. No. 63/092,611, filed on Oct. 16, 2020. The entire contents of the foregoing are incorporated herein by reference.

### FEDERALLY SPONSORED RESEARCH OR DEVELOPMENT

This invention was made with Government support under Grant No. CA214411 awarded by the National Institutes of Health. The Government has certain rights in the invention.

### TECHNICAL FIELD

Described herein are HSP-90 inhibitors conjugated to lipids, and methods of use thereof for treating cancer.

### BACKGROUND

The high mortality in breast cancer is primarily due to most late-stage patients relapsing on chemotherapy and becoming resistant to other drugs (1). This is particularly true in breast cancers that are negative for the cell surface human epidermal growth factor receptor 2 (HER2) and estrogen and progesterone receptors (ER and PR, respectively), known as triple negative breast cancer (TNBC) (2). Indeed, the primary treatment for TNBC includes taxanes alone or combined with anthracyclines (3). Despite some success, recurrence and resistance happens at a substantially higher rate than other breast cancer subtypes, which associates significantly diminished likelihood of survival (4,5). The mechanisms of resistance in TNBC are poorly defined and even emerging modalities such as immunotherapy, in which drugs aim to re-activate immune cells to induce tumor rejection and eradication (6), have yet to markedly enhance duration of response (7-9). Elucidating the drivers and contributors of resistance and identifying modalities to target these mechanisms in TNBC is therefore a critical need towards achieving a sustainable cure.

### SUMMARY

Provided herein are conjugates comprising a heat shock protein 90 (HSP90) inhibitor conjugated to a lipid. In some embodiments, the conjugate is an amphiphile. In some embodiments, the HSP90 inhibitor is radicicol or an analog thereof, e.g., an analog of radicicol selected from KF25706, KF58333, radester, and pochonin D. In some embodiments, the lipid is a cholestanoid (preferably cholesterol), phosphatidylcholine (PC), phosphatidylethanolamine (PE), phosphatidic acid (PA), a phosphatidylserine (PS), or phosphatidylglycerol (PG). In some embodiments, the lipid is cholesterol or phosphatidylcholine (PC).

In some embodiments, the HSP90 inhibitor is conjugated to the lipid via a linker, e.g., as known in the art or described herein. In some embodiments, the linker is selected from the group consisting of: —O—, —S—, —S—S—, —NR¹, —C(O)—, —C(O)O—, —C(O)NR¹, —SO—, —SO₂—, —SO₂NR¹—, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl; wherein one or more methylenes can be interrupted or terminated by O, S, S(O), SO₂, N(R¹)₂, C(O), C(O)O, C(O)NR¹, cleavable linking group, substituted or unsubstituted aryl, substituted or unsubstituted heteroaryl, substituted or unsubstituted heterocyclic, and wherein R¹ is hydrogen, acyl, aliphatic or substituted aliphatic, carbamate, or amide, pH-sensitive, glutathione sensitive, protease sensitive, peptide, disulfide, thioether, and β-glucuronide linkers.

In some embodiments, the linker is C(O), C(O)CH₂CH₂C(O), or C(O)NH(CH₂)₂NHC(O)(CH₂)₂C(O).

In some embodiments, the conjugate has the structure of Formula I or Formula II:

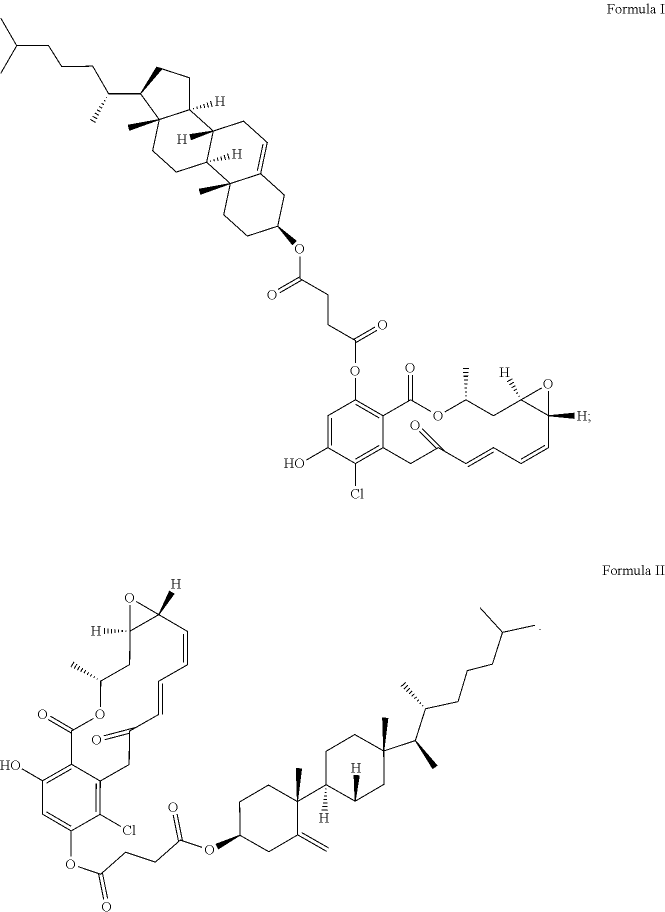

Also provided herein are compositions comprising a conjugate as described herein. In some embodiments, the composition comprises about 1% to about 99% (w/w) of the conjugate. In some embodiments, the composition further comprises an additional lipid in addition to the conjugate. In some embodiments, the composition comprises about 1% to about 99% (w/w) of the additional lipid. In some embodiments, the additional lipid is a lipid conjugated with polyethylene glycol (PEG), optionally wherein the PEG conjugated lipid is selected from the group consisting of PEG conjugated diacylglycerols and dialkylglycerols, PEG-conjugated phosphatidylethanolamine and phosphatidic acid, PEG conjugated ceramides, PEG conjugated dialkylamines, PEG conjugated 1,2-diacyloxypropan-3-amines, and any combinations thereof. In some embodiments, the PEG conjugated lipid is 1,2-distearoyl-sn-glycem-3-phosphoethanolamine-N-[amino(polyethylene glycol)-2000] (DSPE-PEG2000).

In some embodiments, the composition further comprises a phospholipid, preferably wherein the composition comprises about 1%> to about 99% (w/w) of the phospholipid.

In some embodiments, the composition comprises the conjugate and the phospholipid in about 10:1 to about 1:10 ratio, and/or wherein the composition comprises the phospholipid and the lipid in about 10:1 to about 1:10 ratio.

In some embodiments, the phospholipid is selected from phosphatidyl cholines, phosphatidyl cholines with acyl groups having 6 to 22 carbon atoms, phosphatidyl ethanolamines, phosphatidyl inositols, phosphatidic acids, phosphatidyl serines, sphingomyelin, phosphatidyl glycerols, and any combinations thereof, preferably wherein the phospholipid is selected from the group consisting of phosphatidylcholine, phosphatidylglycerol, lecithin, β,γ-dipalmitoyl-a-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, dimyristoyl phosphatidyl choline (DMPC), dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), -phosphatidylethanolamine (POPE), dioleoyl-phosphatidylethanolamine 4-(N-maleimidomethyl)-cyclohexane-1-carboxylate (DOPE-mal), and any combinations thereof.

In some embodiments, the phosphatidylcholine is L-a-phosphatidylcholine.

In some embodiments, the composition further comprises an anticancer agent in addition to the conjugate. In some embodiments, the anticancer agent is a taxane; a platinum compound, an alkylating agent; or an anti-metabolite. In some embodiments, the taxane is paclitaxel.

In some embodiments, the composition comprises the conjugate, a PEG conjugated lipid, and a phospholipid.

In some embodiments, the PEG conjugated lipid is DSPE-PEG2000 and the phospholipid is phosphatidylcholine.

In some embodiments, the composition comprises the conjugate, the PEG conjugated lipid, and the phospholipid in ratio from about 10-0.1:10-0.1:10-0.1, or wherein the ratio is about 1.4:1:3 or about 10:5:1.

In some embodiments, the composition is a nanoparticle, optionally a liposome or polymeric nanoparticle. In some embodiments, the nanoparticle is about 5 nm to about 500 nm in diameter, preferably wherein the nanoparticle 200-300 nm, or about 225 nm, in diameter.

Also provided herein are pharmaceutical compositions comprising a conjugate or composition as described herein, and a pharmaceutically acceptable carrier.

Additionally, provided herein are methods of treating cancer. The methods comprise administering a therapeutically effective amount of a conjugate as described herein to a subject in need thereof. In some embodiments, the methods further include administering an anticancer agent in addition to the conjugate. In some embodiments, the anticancer agent is a taxane; a platinum compound, an alkylating agent; or an anti-metabolite, preferably wherein the taxane is paclitaxel, wherein the anticancer agent is administered before the conjugate. Further, provided herein are methods of treating cancer comprising administering a therapeutically effective amount of a composition as described herein to a subject in need thereof. Also provided are the conjugates and compositions described herein for use in a method of treating cancer in a subject in need thereof.

In some embodiments, the cancer is selected from the group consisting of: breast cancer; ovarian cancer; glioma; gastrointestinal cancer; prostate cancer; carcinoma, lung carcinoma, hepatocellular carcinoma, testicular cancer; cervical cancer; endometrial cancer; bladder cancer; head and neck cancer; lung cancer; gastroesophageal cancer, and gynecological cancer, preferably wherein the cancer is triple negative breast cancer (TNBC).

In some embodiments, the methods described herein further include administering one or more additional anti-cancer therapies to the patient. In some embodiments, the additional therapy is selected from the group consisting of immunotherapy, preferably NK-cell based immunotherapy; surgery; chemotherapy, preferably a taxane; radiation therapy; thermotherapy; hormone therapy; laser therapy; anti-angiogenic therapy; and any combinations thereof; preferably wherein when the additional therapy is NK-cell based immunotherapy, the NK-cell based immunotherapy is administered after the composition or conjugate as described herein

Provided herein are compositions for treating drug resistant cancer cells comprising an Hsp-90 inhibitor-lipid conjugate. In some embodiments, the composition further comprises a taxane, e.g., selected from docetaxel, paclitaxel, abraxane, and cabazitaxel.

In some embodiments, the composition is entrapped or confined in a lipid bilayer, e.g., a bilayer is composed of phosphotidylcholine.

In some embodiments, the inhibitor and lipid are conjugated to each other via linkage selected from a succinate ester linkage, a thioether linkage, a diselenide linkage, a thioketal linkage, an arylboronic ester linkage, an aminoacrylate linkage, an oligoproline linkage, a peroxylate ester linkage, or a mesoporous silicon linkage. In some embodiments, the linkage is a succinate ester linkage. In some embodiments, the linkage further comprises polyethylene glycol as a spacer between the succinate ester and the lipid. In some embodiments, the lipid is selected from cholesterol, phosphatidylcholine (PC), phosphatidylethanolamine (PE), phosphatidic acid (PA), phosphatidylserine (PS), and phosphatidylglycerol (PG).

In some embodiments, the HPS-90 inhibitor is selected from radicicol or derivatives thereof, Ganatespib, BIIB021, 17-AAG (Tanaspemycin), CH5138303, Onalespib, Luminespib, KW-2478, PU—H71, XL888, TAS-116, NMS-E973, and KW2478.

Also provided herein are compositions comprising a taxane and a radicicol-cholesterol conjugate entrapped or confined in a lipid bilayer. In some embodiments, the taxane rapidly releases into cells that uptake the composition prior to the Hsp90 inhibitor and the Hsp90 inhibitor releases slowly into the cells that uptake the composition thereafter. In some embodiments, the composition comprises a larger amount of the Hsp90 inhibitor.

Additionally, provided herein is the use of a composition as described herein to pre-treat a patient having a drug resistant cancer prior to administering a natural killer cell therapy. In some embodiments, the natural killer cell therapy is selected from administration of natural killer cells (NK cells, i.e., CD3⁻ cells), e.g., derived from healthy donor derived peripheral blood, induced pluripotent stem cells (iPSC), umbilical cord stem cells or other natural sources; or NK-92 cells, NK-101 cells or other NK cells obtained and expanded from patients with NK lymphomas; and variants of each of these, which can be genetically modified by chimeric antigen receptors (CAR-NK cells).

Further, provided herein are methods for increasing the number of NKG2D ligand receptors on tumor cells, comprising treating the tumor cells with a composition as described herein, thereby attracting and activating endogenous and adoptive NK cells thereby. Also provided are methods for increasing sensitivity of drug resistant cancer cells to kinase inhibitors or other cancer chemotherapies comprising pre-treating the cancers cells with a composition as described herein.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-I. Drug-induced resistant cancer cells diminish immune surveillance of local NK via release of inhibitory cytokines, in vitro.
(A) Schematic overviews the experimental design to generate drug tolerant cancer cells (DTCCs).
(B) Schematic overviews the experimental design for co-culture of natural killer cells with parental or DTCCs.
(C) Top, cell viability analysis of parental or DTCCs in the presence of varying concentrations of NK92-MI n>9 (left panel) or CD56+ primary human peripheral blood NK n=3 (right panel). Data represent mean±SEM, ***p<0.001, **p<0.01. Bar graph represents mean±SEM. Bottom, Cell viability analysis in parental or DTCCs generated in MDA-MB-468 and SUM159 TNBC cancer cells lines in the presence or absence of the indicated NK:cancer cell co-culture. Bar graph shows mean+/−SEM. *p<0.05 **p<0.01 by t-test, N>3.
(D) Experimental design for 0.2 μm pore-separated co-culture of NK-92MI with parental cells or DTCCs.
(E) Quantification of cell surface biomarkers on NK-92MI following co-incubation with the indicated breast cancer cells for 24 h. N≥3 in biological replicate, bar graph represents mean±SEM, *p<0.05 by t-test.
(F) Schematic overviews the experimental design to isolate cytokines from parental or DTCCs following 4 hour (4 h), 8 hour (8 h) or 24 hour (24 h) culture in fresh media.
(G) Heat map of cytokine expression at different time intervals displayed as the log 2 fold change comparing DTCCs to parental. Hierarchical clustering was performed using Euclidean distance.
(H) Top, cell viability analysis in parental cells co-cultured with NK-92MI cells (1:1 population ratio) in the presence of absence of the indicated cytokines (10 ng/ml) or a cocktail containing: VEGF, G-CSF, GRO, RANTES and IL1a. Cell viability for each cytokine-NK cell combination was normalized to a cancer-only control in which cancer cells were treated with cytokines in the absence of co-culture with NK cells. N=8 in biological replicate, bar graph represents mean±SEM, *p<0.05. Bottom, cell viability analysis in the indicated drug naïve parental cell lines treated with a co-culture with NK92-MI and G-CSF, GM-CSF or vehicle control (10 ng/ml). Data represented as the log 2 fold change comparing the mean of the vehicle control group. Graphs represent the mean+/−SEM, N>3.
(I) Venn diagram shows the top 10 cytokines released by DTCCs vs. parental cells after 24 hour incubation from the indicated cell lines.

FIGS. 2A-H. Hsp90 controls survival and NK cell recognition axes in drug tolerant cancer cells, which can be reversed using radicicol.
(A) Upper panel shows experimental design schematic to study time-dependent protein phosphorylation: parental MDA-MB-231 cells were incubated with a sublethal dose of docetaxel (25 nM) for 0, 4, 12, and 24 h, where zero net proliferation is observed. Lower panels quantify the optical density of protein spots from kinase array studied at different time points used to construct the chemical reaction network (B, C) (N=2 each time point).
(B) Systems biology network interconnects Hsp90 with proteins involved in cell survival, via suppression of caspase-3 (bottom box) or regulation of NK cell recognition via expression of MHC class I polypeptide-related sequence A (MICA), a ligand for NKG2D (top box).
(C) Hsp90 links multiple oncogenic kinases the inhibition of apoptosis (Caspase-3). To model the sensitizing effect of docetaxel, X is included to represent other survival and anti-apoptotic pathways. Connections labeled xA, xH, xE, and βDX represent the effect of docetaxel, and connection labeled αRH represents the effect of radicicol. The naming conventions for the constants are as follows: bₚᵣₒₜₑᵢₙ for production constants, bₚᵣₒₜₑᵢₙ₂ for inhibition scaling constants, dₚᵣₒₜₑᵢₙ for decay constants, k_{reacting protein-activated protein} for reaction constants, α_{reacting protein-inhibited protein} for inhibition constants, xₚᵣₒₜₑᵢₙ for activation by docetaxel, and β_{reacting protein-inhibited protein} for removal of a protein from the model.
(D) Representative western blot showing expression of Hsp90 in MDA-MB-231 parental and DTCCs. N=3 in biological replicate.
(E) Quantification of Hsp90 signal fluorescent intensity in each cell line, expressed as the fold change in relative fluorescence from parental cells. Minimum of 22 individual cells from a minimum of three random fields were quantified from each biological replicate (N=3) per cell line. Bar graph represents mean±SEM, **p<0.01 by t-test.
(F) Quantification of Hsp90 optical density from western blot in FIG. 2D. N=3 per cell line. Bar graph represents mean±SEM, *p<0.05 by t-test.
(G) Representative western blot of phosphorylated HSF-1 (Ser326) evaluated in parental MDA-MB-231 cells under pressure of docetaxel for the indicated amount of time. Blot is representative of similar results from three independent experiments.
(H) Representative western blot analysis of the indicated phosphorylated proteins and total proteins implicated in the Hsp90 pathway, over-expressed in DTCCs compared to parental cells. Blot is representative of similar results from three independent experiments.

FIGS. 3A-P. Sequencing the combination of taxanes and radicicol reduces the proportion of drug tolerant cancer cells and increases NK cell surveillance and cytolysis via NHCA expression in residual populations, in vitro; anticancer efficacy of drug-schedule, in vitro and in silico; effect of Hsp90 inhibitors on NK cell cytolysis
(A) Drug treatment schematic overviews the in vitro approach to sequence docetaxel or radicicol in different, time separated order.
(B) In vitro drug synergy was determined using the Chau-Talalay method. Two schedules of drugs in combination were administered to the indicated TNBC parental cell lines (A). Schedule 1: docetaxel followed by radicicol; Schedule 2: radicicol followed by docetaxel. Plots generated using constant ratio drug combination. Values falling below 1.0 combination index=synergy.
(C) Representative flow cytometry graphs show MICA/B expression in DTCCs treated with vehicle control or radicicol (5 μM) overnight. Values represent the percent (%) positively expressing cells over negative control threshold ±SEM, ***p<0.001. N=11 in biological replicate.
(D) Quantification of MICA/B mean fluorescence, corrected for background by negative control. Data is expressed as the fold change vs. vehicle control and bar graph represents mean±SEM, ***p<0.001. N=11 in biological replicate.
(E) Schematic overviews the experimental design to study the effect of radicicol on NK cytolysis of DTCCs. Note: NK cells are not exposed directly to radicicol in this experimental design.
(F) Quantification of cell viability in DTCCs exposure to radicicol and then co-cultured with NK-92MI (n>10) or primary human NK cells (n=3) in increasing population density. Bar graph represents mean±SEM, ***p<0.001.
(G) Schematic describes the experimental design for NK cytolysis in DTCCs following siRNA gene knockdown of MICA. Note: NK cells are not exposed directly to radicicol in this experimental design.
(H) Quantification of cell viability of MICA depleted DTCCs following the schematic outlined in panel G. Bar graph represents mean±SEM, *p<0.05. N≥4 in biological replicate.
(I) In vitro drug synergy was determined using the Chau-Talalay method. Two schedules of drugs in combination were administered to the indicated TNBC parental cell lines (A). Schedule 1: docetaxel followed by radicicol; Schedule 2: radicicol followed by docetaxel. Plots generated using constant ratio drug combination. Values falling below 1.0 combination index=synergy.
(J) In silico prediction model of drug schedule on signaling pathways. Normalized protein levels are shown for two different treatment schedules [1: Hsp90, 2: SRC, 3: ERK, 4: STAT3, 5: Akt, 6: Caspase-3]. The first drug was administered at the beginning of the experiment (t=0), followed by the second drug after 48 h (t=48). The Docetaxel-Radicicol schedule results in lower normalized protein levels for the “survival proteins” and higher levels for Caspase-3.
(K) Schematic describes the experimental design for panels (L) and (M).
(L) Quantification of cell viability in MDA-MB-231 DTCCs treated as per panel (K). Graph represents mean+/−SEM, N>8, **p<0.01 by ttest.
(M) Quantification of cell viability in SUM 159 DTCCs treated as per panel (K). graph represents mean+/−SEM, N>3. *p<0.05 by t-test.
(N) Schematic describes the experimental design for panel (O)
(O) Quantification of MDA-MB-231 DTCC viability following the treatment schematic outlined in panel (N).
(P) Representative western blot analysis of MICA in MDA-MB-231 DTCCs that were co-incubated with siRNA targeted two unique sequences of MICA. GAPDH loading control.

FIGS. 4A-G. Characterization of a docetaxel-radicicol nanoparticle (DocRad-NP); modeling the effect of free drug docetaxel and radicicol compared to DocRad NPs on Hsp90 related proteins and caspase-3
(A) Schematic of the docetaxel-radicicol nanoparticle (DocRad-NP) structure with respect to the location of docetaxel and radicicol in the lipid bilayer.
(B) Structural schematic to illustrate the synthesis of the radicicol-cholesterol compound which is inserted into the lipid bilayer of the nanoparticle.
(C) Quantification and distribution of the hydrodynamic diameter of DocRad-NP Histogram is representative of three independent experiments producing similar results.
(D) Quantification of physical stability of DocRad-NP on storage at 4° C. measured as the difference in Zeta potential (mV, squares) and size (nm, circles). Line graph is representative of three independent experiments producing similar results.
(E) Release kinetics of Docetaxel and Radicicol from DocRad-NP in PBS (pH 7.4) or 4T1 mammary carcinoma cell lysate. N=3 in biological replicate. *p<0.05 comparing the % radicicol release and docetaxel release at the indicated time point.
(F) Normalized protein levels are compared for free drug administration (solid lines) and nanoparticles (dashed lines) for docetaxel and radicicol. For docetaxel and radicicol together, the experiments showed that the delayed release of radicicol in the nanoparticles (DocRad NP) took greater advantage of the sensitizing effect of docetaxel.
(G) Normalized protein levels are compared for free drug administration (solid lines) and nanoparticles (dashed lines) for radicicol only. For free drug radicicol, the experiments showed that the effect of the nanoparticles is slightly lessened compared to the free drug but with a longer lasting effect. In both experiments, both the free drug and nanoparticle treatments administer the same total amount of drug, however radicicol is released slowly due to conjugation to cholesterol.

FIGS. 5A-G. In vitro characterization of radicicol nanoformulation confirms increased anticancer effect, sustained inhibition of Hsp90-related survival axis and enhanced MICA/B expression, as compared to the free drug radicicol.
(A) Schematic overviews the in vitro experimental design DTCCs transiently treated with radicicol or Rad-NP
(B) Western blot analysis of DTCCs following transient exposure to Rad-NP or radicicol free drug, as described in (A).
(C) Cell viability analysis of DTCCs derived from several luminal or TNBC cell lines following constant treatment with indicated concentration-equivalent doses of single-loaded NP and the dual loaded DocRad-NP.
(D) Quantification of MICA/B expression on DTCCs after a transient exposure (4 hours) with radicicol free drug or equivalent dose of Rad-NP and following washout and incubation for an additional 20 hours (read out at 24 hours total incubation). N>3 in biological replicate Bar graph represents mean±SEM, ***p<0.001 by one-way ANOVA, N=3 in biological replicate.
(E) Quantification of MICA/B fluorescence in the indicated treatment conditions by flow cytometry. Bar graph represents mean±SEM, ***p<0.001 by one-way ANOVA, n=3 in biological replicate.
(F) Quantification of cell number following exposure to docetaxel nanoparticle (Doc-NP) or chimeric nanoparticle (DocRad-NP) for 48 hours and sequential administration of NK-92MI (24 hours).
(G) Schematic summarizes the effect of Rad-NP compared to free drug based on empirical data.

FIGS. 6A-F. DocRad-NP reduces tumor burden, sustains inhibition of pro-survival proteins and primes residual tumor cells for NK surveillance via upregulation of the NKG2D ligand receptor, MULT-1, in vivo; in vivo toxicity analysis.
(A-D) Orthotopic syngeneic mammary carcinoma model (4T-1) receiving the following treatments: vehicle, docetaxel, radicicol, docetaxel and radicicol, or a 2-in-1 docetaxel radicicol nanoparticle (DocRad-NP) delivered at equivalent doses. N=4 per group. Immunohistochemistry (IHC) images were determined by a clinical pathologist blinded to the treatment condition as a representation of the overall effect of treatment from each treatment group.
(A) Quantification of tumor growth curves from Arrows indicate specific days the mice were treated. (Top) Representative tumors from mice harvested at the end of treatment. **p<0.01 by two-way ANOVA (Doc+rad vs. DocRad NP). Animals were treated with docetaxel on days 1 and 3 (blue arrows). Black arrows indicate subsequent treatment regimens.
(B) Representative confocal microscopy shows fluorescence intensity of TUNEL (indication of apoptosis). Scale bar=120 μm.
(C) Representative images from IHC. Scale bar=75 μm.
(D) Representative images from IHC. Inset of magnified representative section to show staining distribution and intensity. Scale bar=75 μm.
(E) Representative images from IHC serial sections of the same tissue region. Scale bar=75 μm.
(F) Body weight was analyzed over the course of treatment. Arrows indicate days when drugs were administered.

FIGS. 7A-H. Confirmation of a dynamic role for tumor infiltrated NK cells in drug-induced cancer cell death using human TNBC samples.
(A-F) Ex vivo human tumor model system used to study spatial distribution of natural killer (NK) cells in the tumor and stroma under drug pressure. All tissue biopsies are from triple negative breast cancer patients, n=7 patient samples, fragments from each patient biopsy are plated into triplicate fragments per treatment ‘arm’. Patient demographic and metadata can be found in supplemental data files.
(A) Schematic overviews the ex vivo tumor model, comprising live human tissue fragments from biopsy plated into culture wells and treated with vehicle or drug as described in methods. Image was reproduced with permission. Inky Mouse Studios, 2018 all rights reserved.
(B) Schematic overviews the analytical process of using thin-cut serial FFPE sections to discern tumor vs. stroma (H&E), drug-induced cell death via immunohistochemistry (IHC) of apoptosis (cleaved caspase-3) and overlay multiplex IHC (mIHC) for identification of natural killer cells PanCK⁻CD3⁻ CD56⁺.
(C) Representative mIHC overviews the strategy to identify and quantify the spatial arrangement of NK cells (teal) vs. T-cells (red) in the stroma via measurement of distance to the tumor interface (red line; Dₜ).
(D) Quantification of cleaved caspase-3 presented as a waterfall plot. Histogram represents the log 2 fold change of drug vs. vehicle. A cut-off of 0.5 demarcated by the dashed line separates samples as caspase-3 Hi (black bars) vs. Lo (grey bars).
(E) Spearman correlation rank order heatmap. Five various cellular localization, density and spatial arrangement metrics were analyzed for correlation within ‘caspase-3 Lo’ and ‘caspase-3 Hi’ samples. Positive correlations are displayed in red and negative correlations in blue color. Color intensity and the size of the circle are proportional to the correlation coefficients.
(F) Quantification of NK cell density and spatial arrangement represented as the log 2 fold change of drug vs. vehicle. Bar graph represents mean±SEM, *p<0.05.
(G) Representative tissue microarray (TMA) of hematoxylin and eosin (H&E) staining shows how a typical experiment is performed from a single patient tumor biopsy. Drug treatment is performed in triplicate per treatment ‘arm’ (box illustrates a single example drug ‘arm’).
(H) Representative experimental workflow for assessing caspase-3 activity. Identification of tumor area bis H&E, serial section 4 mm slices from formalin fixed paraffin embedded (FFPE) is then performed to stain for cleaved caspase-3 by immunohistochemistry (IHC). HALO is deployed to quantify the expression of cleaved caspase-3. Three independent tumor regions are assessed per tissue fragment and normalized to total number of cells. Expression levels in drug treatment is subtracted from the vehicle control ‘arms’ to provide a final value of drug-induced cleaved caspase-3.

FIGS. 8A-I. Lipid-based targeting of drug tolerant cancer cells (DTCCs).
A. Schematic representation of the experimental strategy for the generation of acute drug tolerant cancer cells (DTCCs) in vitro. B. Representative florescent microscopy image of the lipid raft formation in drug naïve cancer cells (DNCCs) and DTCCs. Lipid raft labeling (red) and DAPI (blue). Scale bar=10 μm Right panel shows 20×image, scale bar=3 μm. C. The experimental workflow of the lipid screening strategy. D. Histogram shows the normalized uptake of the lipid raft-targeting agents in DNCCs and in DTCCs. The fluorescent intensity obtained in each case has been recorded and normalized according to the DNCC fluorescence intensity. **p<0.01 ****p<0.0001 by t-test, N>3 in biological replicate. E. Trace shows uptake of NBD-PC [5 μM] into MDA-MB-231 DTCCs or DNCCs over time. Units are in arbitrary fluorescence as determined by flow cytometry, N=3 in biological replicate. F. Representative image showing the colocalization of lipid rafts and NBD-PC or NBD-cholesterol. DTCCs were incubated with NBD-PC/Cholesterol and stained with lipid raft labeling agent. This data demonstrates the internalization of the NBD-PC lipid across the lipid raft present on the cellular membrane performed in biological replicates. Scale bar=10 μm. G. Heatmap shows the change in fluorescence intensity of DTCCs relative to DNCCs from two TNBC cell lines (log 2 fold change) as determined by flow cytometry. Arrows indicate lipids with increased uptake in DTCCs of both cell lines tested. H. Structures of lipids used for the lipid screening assay. I. Kinetics of internalization of fluorescent lipid in DTCC and DNCC. NBD PC was added to the DNCC and DTCC and the amount of internalization has been checked at definite time interval. Data shows the higher rate of internalization of NBD-PC in case of DTCC.

FIG. 9. Chemical reaction schemes for radicicol conjugate.

### DETAILED DESCRIPTION

Intratumor heterogeneity, cancer stem cells (CSC) and mutational evolution have long been implicated as the drivers of both intrinsic and acquired drug resistance (10). An emerging paradigm, however, is drug-induced resistance, or tolerance, which has been described as phenotypic transitions within subpopulations of cancer cells in the presence of drugs(11), which we previously showed can arise from non-CSC via protein expressions, kinase scaffolding and signaling activations (12). The heat shock protein 90 (Hsp90) plays a broad role in cellular signaling, including a direct effect on protein kinases, operating as an ATP-dependent dimeric molecular chaperone to form the core of large complexes with cochaperones and substrates (13). Indeed, combinations of Hsp90 inhibitors and chemotherapies have been studied (14) with the goal of targeting multiple pro-survival pathways including signal transducer and activator of transcription (STAT), extracellular signal regulated kinases (ERK), Src family kinases (SFK) and Phosphoinositide 3-kinases (PI3K) families of proteins, which are augmented under external stress(15). However, targeting Hsp90 in clinical studies has been somewhat lackluster(16) suggesting novel approaches that deploy rational combinations of drugs could help to address the existing challenges.

A concerted effort to understand the biological interaction between tumor, stroma and immune cells within the tumor immune microenvironment (TIME) will contribute to clinical treatment success (17). Not only are the activity and exhaustion status of cytolytic immune cells, such as CD8+ cytotoxic T-cells and natural killer (NK) cells, implicated in tumor rejection (18), their spatial arrangement and locations within the tumor are critical for prognostic benefit of anticancer cytotoxics and cancer immunotherapies(19). Attempts to improve tumor surveillance via augmenting immune cell activities (20) or suppressing the ‘don’t eat me signals' on tumor cells have been tested (21). Few studies, however, have sought to increase tumor cell surface ligands that invigorate NK or T-cell surveillance such as MHC class I polypeptide-related sequence A, B (MICA/B) (22) to ‘unmask’ tumors from immune-evasion.

Resolving drug resistance is penultimate to finding a sustainable cure for cancer. While conventional models of drug resistance rely on stochastic mutations conferred through Darwinian evolution, drug-induced resistance is seen as a measure of cellular ‘fitness’ wherein the entirety of the tumor ecosystem contributes to the effect while under drug pressure. There is a paucity of literature to support how drug-induced resistant cancer cells and other cells, such as NK, ‘cooperate’ or ‘compete’ to drive tumor growth. We focused on the role of innate NK in drug-induced resistance and based on this information we engineered potential therapeutic strategies and established Hsp90 as a putative ‘lynch pin’ in the survival signaling pathway while simultaneously ‘putting the brakes’ on the surveillance of NK cells for tumor cell clearance. To some degree, we relied on the systems biology model to establish the shortest molecular relationship among this effect. While this simplified the protein interactions involved and may overestimate the effect of Hsp90 due to its simplicity, it provided the necessary evidence that the effect of Hsp90 on Src, ERK, STAT3, and Akt are significantly changing the cell's response to docetaxel while simultaneously depressing innate immune surveillance.

This study interrogated the TIME in drug-induced resistance and the role that chaperones contribute as druggable targets in this effect. Using in vitro co-culture experiments molecular and computational screening approaches, cancer nanomedicines as a tool and in vivo translational models, a tumor-targeted, engineered therapeutic approach was developed that re-invigorates or reawakens NK cells to combat resistance phenotypes that emerge under drug pressure.

The present results were surprising because Hsp-90 inhibitors have been shown to actually de-activate NK cells. See, e.g., Bae et al., J Immunol. 2013 Feb. 1; 190(3):1360-71 (See also FIGS. 3N-O, where Hsp90 inhibition alone depressed the function of NK cells against tumor cells). These findings demonstrate that it is the bioengineered NP that targets the tumor and avoids inhibition of the NK cells by releasing the payload within the cancer cells themselves. This is shown by the immunohistochemistry and the confocal microscopy (FIG. 6) results. There was preferential uptake into the tumor cells which avoids harming the NK cells. Thus, it is counter intuitive that an Hsp90 inhibitor NP would “re-awaken” or at the very least, not harm the NK cells. Also surprising is the incorporation efficiency as compared to the NPs exemplified in U.S. Pat. No. 10,300,143. In that patent, the incorporation efficiency was in the range of 40 percent for PI-828 and 55-65% for PI-103, whereas in the molecule(s) exemplified herein it was 80-90%.

Compositions

Described herein are Hsp-90 inhibitor-lipid conjugates, compositions comprising the Hsp-90 inhibitor-lipid conjugates, and methods of using them, e.g., for treating drug tolerant cancer cells (DTCCs) and cancers that have become drug tolerant, or for reducing the risk that a cancer will become drug tolerant.

Such compositions comprise an Hsp90 inhibitor-lipid conjugate, comprising an HSP90 inhibitor covalently linked to a lipid, optionally via a linker.

HSP90 Inhibitors

The term “HSP90 inhibitors”, as used herein, includes, but is not limited to, compounds targeting, decreasing or inhibiting the intrinsic ATPase activity of HSP90; degrading, targeting, decreasing or inhibiting the HSP90 client proteins via the ubiquitin proteasome pathway. Compounds targeting, decreasing or inhibiting the intrinsic ATPase activity of HSP90 are especially compounds, proteins or antibodies that inhibit the ATPase activity of HSP90, e.g., 17-allylamino, 17-demethoxygeldanamycin (17-AAG), 17-DMAG (Alvespimycin), IPI-504 (17-AAG Hydroquinone; Retaspamycin), IPI-493 (17-AG), Macbecin and other geldanamycin derivatives; other geldanamycin-related compounds; radicicol inhibitors; and radicicol analogs that bind and inhibit HSP90 (e.g., radester, pochonin D, oxime- (e.g., KF25706 and KF58333), cyclopropyl- and cyclopropane-analogues, zearalenol, and other macrolactams, e.g., as described in Dutton et al., Org. Biomol. Chem., 2014, 12, 1328-1340). In some embodiments, the HPS-90 inhibitor can be radicicol, ganatespib, BIIB021, 17-AAG (Tanaspemycin), CH5138303, onalespib, luminespib, KW-2478, PU—H71, XL888, TAS-116, NMS-E973, TAS-116, or KW2478. See also the HSP90 inhibitory compounds described in Wang et al., J. Med. Chem. 2016, 59, 12, 5563-5586; Sidera and Patsavoudi, Recent patents on Anti-Cancer Drug Discovery, 2014, 9, 1-20.

Linkers

The inhibitor can be conjugated to the lipid via a linkage, for example via a succinate ester linkage, a thioether linkage, a diselenide linkage, a thioketal linkage, an arylboronic ester linkage, an aminoacrylate linkage, an oligoproline linkage, a peroxylate ester linkage, or a mesoporous silicon linkage. In some embodiments, the linker is selected from the group consisting of: —O—, —S—, —S—S—, —NR—, —C(O)—, —C(O)O—, —C(O)NR¹—, —SO—, —SO₂—, —SO₂NR¹—, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl; wherein one or more methylenes can be interrupted or terminated by O, S, S(O), SO₂, N(R¹)₂, C(O), C(O)O, C(O)NR¹, cleavable linking group, substituted or unsubstituted aryl, substituted or unsubstituted heteroaryl, substituted or unsubstituted heterocyclic, and wherein R¹ is hydrogen, acyl, aliphatic or substituted aliphatic. In some embodiments, the linker is C(O), C(O)CH₂CH₂C(O), or C(O)NH(CH₂)₂NHC(O)(CH₂)₂C(O). The linkage may further comprise one or more polyethylene glycol moieties as a spacer between the linkage, for example the succinate ester, and the lipid. Methods known in the art can be used to modify the HSP90 inhibitor to add a linker.

TABLE A
Exemplary linkers

| ROS- | |
|---|---|
| sensitive | |
| linkers | Chemical structure and oxidation |
| Thioether, selenide and telluride (solubility change) | 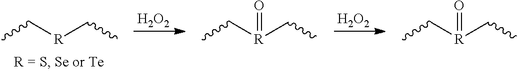 |
| Thioether, selenide and telluride (cleavage) | 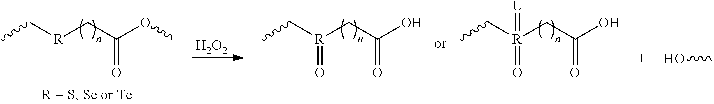 |
| Diselenide | 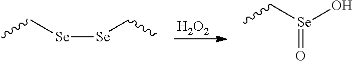 |
| Thioketal | 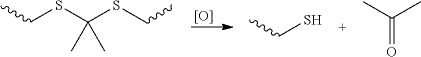 |
| Arylboronic ester | 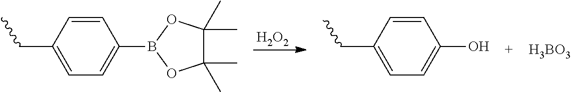 |
| Amino- acrylate | 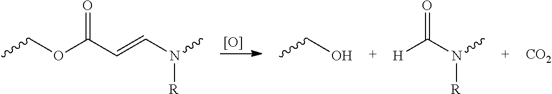 |
| Oligoproline | 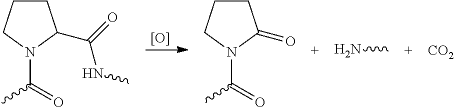 |
| Peroxalate ester | 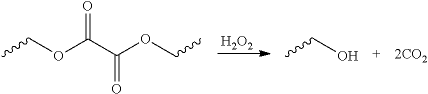 |
| Mesoporous silicon | 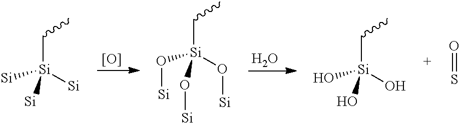 |

In some embodiments, the linker comprises at least one cleavable linking group; see, e.g., U.S. Pat. No. 9,789,193.

In preferred embodiments, the linker provides slow release of the active molecule from the nanoparticle. Exemplary linkers for slow release include sulfatase linkers (see, e.g., Bargh et al., Chem. Sci., 2020, 11, 2375-2380); carbamate linkers; amide linkers; glutathione sensitive linkers, e.g., SPDB (SPP); protease sensitive linkers; pH sensitive linkers;

In some embodiments, the linker comprises a pH-sensitive linker that is sensitive to hydrolysis at certain pH values. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661) could be used. Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929). In some embodiments, the linker is a malonate linker (Johnson et al., 1995, Anticancer Res. 15:1387-93), a maleimidobenzoyl linker (Lau et al., 1995, Bioorg-Med-Chem. 3(10): 1299-1304), a maleimidocaproyl (“mc”) linker (Doronina et al., 2006, Bioconjug Chem. 17:114-24), or a 3′-N-amide analog (Lau et al., 1995, Bioorg-Med-Chem. 3(10):1305-12). Peptide (e.g., valine-citrulline (Val-Cit) dipeptide linker, Glutamic acid-valine-citrulline (Glu-Val-Cit) linker, phenylalanine-lysine (Phe-Lys) dipeptide linker) and hydrazine linkers can also be used. See, e.g., US 20060024317; US 20100092496; US 20150079114; and WO 2009143412.

The conjugates described herein can include linkers comprising a disulfide bridge. The linkers can comprise functional or reactive moieties capable of covalently binding to a lipid and an HSP90i. Exemplary functional groups include hydroxyl, amine, thiol, carboxyl, aldehyde, glyoxal, dione, alkenyl, alkynyl, alkedienyl, azide, acrylamide, vinyl sulfone, hydrazide, aminoxy, maleimide, dithiopyridine, and iodoacetamide moieties. The linkers can further include a C₁₋₂₀ alkyl on either side of the disulfide bridge. The alkyl chain can be linear or branched, saturated or unsaturated, unsubstituted or substituted. For example, the linkers can have a general formula: X₁-L₁-S—S-L₂-X₂ wherein: X₁ and X₂ are each independently a functional or reactive moiety as described above (e.g., hydroxyl, amine, thiol, carboxyl, aldehyde, glyoxal, dione, alkenyl, alkynyl, alkedienyl, azide, acrylamide, vinyl sulfone, hydrazide, aminoxy, maleimide, dithiopyridine, and iodoacetamide moieties); L₁ and L₂ are each independently a C₁₋₂₀ alkyl; and S—S is a disulfide bridge.

A variety of other disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935). See, e.g., US 20060024317; US 20100092496; US 20150079114; and WO 2009143412, all of which are incorporated herein in their entirety. In some embodiments, the linkers are maleimide linkers, e.g., as described in Doronin et al., Bioconjugate Chemistry 2006, 17, 1, 114-124 (e.g., maleimidocaproyl-valine-citrulline-p-aminobenzyloxycarbonyl (maleimidocaproyl-Val-Cit-PABC); maleimidocaproyl-Val-Cit; maleimidocaproyl-PABC; or maleimidocaproyl) or β-glucuronide linkers (e.g., as described in Ravasco et al., Chem. Eur. J. 2019, 25:43 and Jeffrey et al., ACS Med Chem Lett. 2010 Sep. 9; 1(6): 277-280, optionally comprising dimethylethylene diamine (DMED)).

See also the linkers described in U.S. Pat. No. 10,300,070; and Irby et al., Mol Pharm. 2017 May 1; 14(5): 1325-1338; Shrivastava et al., Curr Pharm Des. 2020; 26(27):3187-3202; Adhikari et al., Int J Pharm. 2017 Aug. 30; 529(1-2):629-641; Banerjee and Kundu, Daru. 2018 Sep.; 26(1):65-75; and Heyes et al., Journal of Controlled Release 112 (2006) 280-290.

Lipids

The term “lipid” as used herein means a substance that is soluble in organic solvents and includes, but is not limited to, oils, fats, sterols, triglycerides, fatty acids, phospholipids, and the like.

Without limitations the lipid can be selected from the group consisting of sterol lipids, fatty acids, fatty alcohols, glycerolipids (e.g., monoglycerides, diglycerides, and triglycerides), phospholipids, glycerophospholipids, sphingolipids, prenol lipids, saccharolipids, polyketides, and any combination thereof. The lipid can be a polyunsaturated fatty acid or alcohol. The term “polyunsaturated fatty acid” or “polyunsaturated fatty alcohol” as used herein means a fatty acid or alcohol with two or more carbon-carbon double bonds in its hydrocarbon chain. The lipid can also be a highly unsaturated fatty acid or alcohol. The term “highly polyunsaturated fatty acid” or “highly polyunsaturated fatty alcohol” as used herein means a fatty acid or alcohol having at least 18 carbon atoms and at least 3 double bonds. The lipid can be an omega-3 fatty acid. The term “omega-3 fatty acid” as used herein means a polyunsaturated fatty acid whose first double bond occurs at the third carbon-carbon bond from the end opposite the acid group.

In some embodiments, the lipid can be selected from the group consisting of cholesterol; 1,3-Propanediol Dicaprylate/Dicaprate; 10-undecenoic acid; 1-dotriacontanol; 1-heptacosanol; 1-nonacosanol; 2-ethyl hexanol; Androstanes; Arachidic acid; Arachidonic acid; arachidyl alcohol; Behenic acid; behenyl alcohol; Capmul MCM C10; Capric acid; capric alcohol; capryl alcohol; Caprylic acid; Caprylic/Capric Acid Ester of Saturated Fatty Alcohol C12-C18; Caprylic/Capric Triglyceride; Caprylic/Capric Triglyceride; Ceramide phosphorylcholine (Sphingomyelin, SPH); Ceramide phosphorylethanolamine (Sphingomyelin, Cer-PE); Ceramide phosphorylglycerol; Ceroplastic acid; Cerotic acid; Cerotic acid; ceryl alcohol; Cetearyl alcohol; Ceteth-10; cetyl alcohol; Cholanes; Cholestanes; cholesterol; cis-11-eicosenoic acid; cis-11-octadecenoic acid; cis-13-docosenoic acid; cluytyl alcohol; Dihomo-.gamma.-linolenic; Docosahexaenoic acid; egg lecithin; Eicosapentaenoic acid; Eicosenoic acid; Elaidic acid; elaidolinolenyl alcohol; elaidolinoleyl alcohol; elaidyl alcohol; Erucic acid; erucyl alcohol; Estranes; Ethylene glycol distearate (EGDS); Geddic acid; geddyl alcohol; glycerol distearate (type I) EP (Precirol ATO 5); Glycerol Tricaprylate/Caprate; Glycerol Tricaprylate/Caprate (CAPTEX 355 EP/NF); glyceryl monocaprylate (Capmul MCM C8 EP); Glyceryl Triacetate; Glyceryl Tricaprylate; Glyceryl Tricaprylate/Caprate/Laurate; Glyceryl Tricaprylate/Tricaprate; glyceryl tripalmitate (Tripalmitin); Henatriacontylic acid; Heneicosyl alcohol; Heneicosylic acid; Heptacosylic acid; Heptadecanoic acid; Heptadecyl alcohol; Hexatriacontylic acid; isostearic acid; isostearyl alcohol; Lacceroic acid; Laurie acid; Lauryl alcohol; Lignoceric acid; lignoceryl alcohol; Linoelaidic acid; Linoleic acid; linolenyl alcohol; linoleyl alcohol; Margaric acid; Mead; Melissic acid; melissyl alcohol; Montanic acid; montanyl alcohol; myricyl alcohol; Myristic acid; Myristoleic acid; Myristyl alcohol; neodecanoic acid; neoheptanoic acid; neononanoic acid; Nervonic; Nonacosylic acid; Nonadecyl alcohol; Nonadecylic acid; Nonadecylic acid; Oleic acid; oleyl alcohol; Palmitic acid; Palmitoleic acid; palmitoleyl alcohol; Pelargonic acid; pelargonic alcohol; Pentacosylic acid; Pentadecyl alcohol; Pentadecylic acid; Phosphatidic acid (phosphatidate, PA); Phosphatidylcholine (lecithin, PC); Phosphatidylethanolamine (cephalin, PE); Phosphatidylinositol (PI); Phosphatidylinositol bisphosphate (PIP2); Phosphatidylinositol phosphate (PIP); Phosphatidylinositol triphosphate (PIP3); Phosphatidylserine (PS); polyglyceryl-6-distearate; Pregnanes; Propylene Glycol Dicaprate; Propylene Glycol Dicaprylocaprate; Propylene Glycol Dicaprylocaprate; Psyllic acid; recinoleaic acid; recinoleyl alcohol; Sapienic acid; soy lecithin; Stearic acid; Stearidonic; stearyl alcohol; Tricosylic acid; Tridecyl alcohol; Tridecylic acid; Triolein; Undecyl alcohol; undecylenic acid; Undecylic acid; Vaccenic acid; alpha-Linolenic acid; and gamma-Linolenic acid.

In some embodiments, the lipid is cholesterol. In some embodiments, the cholesterol can further comprise succinate and/or succinic acid for conjugating with the chemotherapeutic agent.

In preferred embodiments, the lipid can be, for example, a cholesterol (or any other cholestanoid, i.e., any steroid based on a cholestane skeleton and its derivatives, e.g., C27 bile acids), a phosphatidylcholine (PC), a phosphatidylethanolamine (PE), a phosphatidic acid (PA), a phosphatidylserine (PS), or a phosphatidylglycerol (PG) (see, e.g. FIG. 8H). In preferred embodiments, the lipids show preferential uptake into drug resistant cancer cells, i.e., cancer cells that have been previously exposed to a chemotherapy and that no longer respond to that chemotherapy.

Compositions

Also described herein are compositions comprising a conjugate as described herein. In some embodiments, the composition comprises about 1% to about 99% (w/w) of the conjugate. In some embodiments, the composition further comprises a lipid in addition to the conjugate, e.g., a bilayer- or particle-forming lipid. In some embodiments, the composition comprises about 1% to about 99% (w/w) of the additional lipid. In some embodiments, the composition comprises the conjugate and the additional lipid in about 10:1 to about 1:10 ratio. In some embodiments, the additional lipid is a lipid conjugated with polyethylene glycol (PEG). In some embodiments, the PEG conjugated additional lipid is selected from the group consisting of PEG conjugated diacylglycerols and dialkylglycerols, PEG-conjugated phosphatidylethanolamine and phosphatidic acid, PEG conjugated ceramides, PEG conjugated dialkylamines, PEG conjugated 1,2-diacyloxypropan-3-amines, and any combinations thereof. In some embodiments, the PEG conjugated additional lipid is 1,2-distearoyl-sn-glycem-3-phosphoethanolamine-N-[amino(polyethylene glycol)-2000] (DSPE-PEG2000).

In some embodiments, the composition further comprises a phospholipid. In some embodiments, the composition comprises about 1% to about 99% (w/w) of the phospholipid. In some embodiments, the composition comprises the conjugate and the phospholipid in about 10:1 to about 1:10 ratio. In some embodiments, the composition comprises the phospholipid and the lipid in about 10:1 to about 1:10 ratio. In some embodiments, the phospholipid is selected from phosphatidyl cholines, phosphatidyl cholines with acyl groups having 6 to 22 carbon atoms, phosphatidyl ethanolamines, phosphatidyl inositols, phosphatidic acids, phosphatidyl serines, sphingomyelin, phosphatidyl glycerols, and any combinations thereof. In some embodiments, the phospholipid is selected from the group consisting of phosphatidylcholine, phosphatidylglycerol, lecithin, beta,gamma-dipalmitoyl-alpha-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, dimyristoyl phosphatidyl choline (DMPC), dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), -phosphatidylethanolamine (POPE), dioleoyl-phosphatidylethanolamine 4-(N-maleimidomethyl)-cyclohexane-1-carboxylate (DOPE-mal), and any combinations thereof. In some embodiments, the phosphatidylcholine is L-a-phosphatidylcholine.

In addition to the conjugate, the composition (e.g., a particle comprising the conjugate) can further include one or more additional lipids and/or other components such as cholesterol. Without wishing to be bound by a theory, other lipids can be included in the compositions for a variety of purposes, such as to prevent lipid oxidation, to stabilize bilayer, to reduce aggregation during formation or to attach ligands onto the particle surface. Any of a number of lipids can be present, including but not limited to, amphipathic, neutral, cationic, anionic lipids, sterols, and phospholipids. Further, such lipids can be used alone or in any combination with each other. In some embodiments, the composition further comprises a lipoprotein particle, e.g., HDL or LDL. The composition can comprise from about 1% to about 99% (w/w) of the additional lipid or component. Further the additional lipid or component can be present in 10:1 to 1:10 ratio with the conjugate. If two or more different additional lipids are present in the composition, each lipid can be independently in 10:1 to 1:10 ratio with the conjugate. Further, if two or more different additional lipids are present in the composition, the two lipids can be in 10:1 to 1:10 ratio. Without limitations, two different components (conjugate and lipid or two different lipids) of the composition can be in ratio 10:1 to 1:10, 5:1 to 1:5, or 2.5:1 to 1:2.5. In some embodiments, two different components in the composition can be in ratio of about 1:1, about 1:1.2, about 1:1.5, about 1:1.7, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, or about 1:10. If the composition comprises more than two components ratio between any two components can be independent of ratio between any other two components.

Additional components that can be present in the particle composition can include bilayer stabilizing components such as polyamide oligomers (see, e.g., U.S. Pat. No. 6,320,017), peptides, proteins, detergents, lipid-derivatives, such as PEG conjugated to phosphatidylethanolamine, PEG conjugated to phosphatidic acid, PEG conjugated to ceramides (see, U.S. Pat. No. 5,885,613), PEG conjugated dialkylamines, PEG conjugated 1,2-diacyloxypropan-3-amines, and PEG conjugated to 1,2-distearoyl-sn-glycem-3-phosphoethanolamine (DSPE). In some embodiments, the bilayer stabilizing component is DSPE-PEG2000.

The composition can also include components selected to reduce aggregation of particles during formation, which can result from steric stabilization of particles which prevents charge-induced aggregation during formation. Suitable components that reduce aggregation include, but are not limited to, polyethylene glycol (PEG)-modified lipids (i.e., PEG conjugated lipids), monosialoganglioside Gm1, and polyamide oligomers (“PAO”) such as (described in U.S. Pat. No. 6,320,017). Exemplary suitable PEG-modified lipids include, but are not limited to, PEG-modified diacylglycerols and dialkylglycerols, PEG-modified phosphatidylethanolamine and phosphatidic acid, PEG-ceramide conjugates (e.g., PEG-CerC14 or PEG-CerC20), PEG-modified dialkylamines, PEG-modified 1,2-diacyloxypropan-3-amines, and PEG conjugated DSPE (e.g., DSPE-PEG2000). Other compounds with uncharged, hydrophilic, steric-barrier moieties, which prevent aggregation during formation, like PEG, Gm1, or ATTA, can also be coupled to lipids to reduce aggregation during formation. ATTA-lipids are described, e.g., in U.S. Pat. No. 6,320,017, and PEG-lipid conjugates are described, e.g., in U.S. Pat. Nos. 5,820,873, 5,534,499 and 5,885,613. Typically, the concentration of the lipid component selected to reduce aggregation is about 0.1 to 15% (by mole percent of lipids). It should be noted that aggregation preventing compounds do not necessarily require lipid conjugation to function properly. Free PEG or free ATTA in solution can be sufficient to prevent aggregation. If the liposomes are stable after formulation, the PEG or ATTA can be dialyzed away before administration to a subject.

Neutral lipids, when present in the composition, can be any of a number of lipid species which exist either in an uncharged or neutral zwitterionic form at physiological pH. Such lipids include, but are not limited to, diacylphosphatidylcholine, diacylphosphatidylethanolamine, ceramide, sphingomyelin, dihydrosphingomyelin, cephalin, and cerebrosides. The selection of neutral lipids for use in liposomes described herein is generally guided by consideration of, e.g., liposome size and stability of the liposomes in the bloodstream. Preferably, the neutral lipid component is a lipid having two acyl groups, (i.e., diacylphosphatidylcholine and diacylphosphatidylethanolamine) Lipids having a variety of acyl chain groups of varying chain length and degree of saturation are available or can be isolated or synthesized by well-known techniques. In one group of embodiments, lipids containing saturated or unsaturated fatty acids with carbon chain lengths in the range of C₆ to C₂₂ (e.g., C₆, C₈, C₁₀, C₁₂, C₁₄, C₁₆, C₁₈, C₂₀, or C₂₂) are preferred. Additionally, lipids having mixtures of saturated and unsaturated fatty acid chains can be used. In some embodiments, the neutral lipids can be phosphatidylcholine, DOPE, DSPC, POPC, DMPC, DPPC or any related phosphatidylcholine. The neutral lipids useful in the present invention can also be composed of sphingomyelin, dihydrosphingomyeline, or phospholipids with other head groups, such as serine and inositol.

When present in the composition, the sterol component can be any of those sterols conventionally used in the field of liposome, lipid vesicle or lipid particle preparation. A preferred sterol is cholesterol.

When present in the composition, the cationic lipids can be any of a number of lipid species which carry a net positive charge at about physiological pH. Such lipids include, but are not limited to, N,N-dioleyl-N,N-dimethylammonium chloride (“DODAC”); N-(2,3-dioleyloxyl)propyl-N,N—N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3.beta.-(N—(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N, N-dimethyl-2,3-dioleyloxy)propylamine (“DODMA”), N-(1,2-dimyristyloxyprop-3-yl)-N,N-dimethyl-N-hydroxyethyl ammonium bromide (“DMRIE”), 5-carboxyspermylglycine diocaoleyamide (“DOGS”), and dipalmitoylphosphatidylethanolamine 5-carboxyspermyl-amide (“DPPES”). Additionally, a number of commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL). Other cationic lipids suitable for lipid particle formation are described in WO98/39359, WO96/37194. Other suitable cationic lipids are described, for example in US Patent Application Publication No. 2011/0997720 and PCT Patent Application Publication No. WO 2009/132131 and No. WO 2009/132131, content of all of which is incorporated herein by reference in its entirety.

When present in the composition, the anionic lipid can be any of a number of lipid species which carry a net negative charge at about physiological pH. Such lipids include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

As used herein, the term “amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids.

In some embodiments, the composition can further comprise a targeting agent. In some embodiments, the targeting agent is selected from the group consisting of peptides, polypeptides, proteins, enzymes, peptidomimetics, glycoproteins, antibodies (monoclonal or polyclonal) and portions and fragments thereof, lectins, nucleosides, nucleotides, nucleoside and nucleotide analogues, nucleic acids, monosaccharides, disaccharides, trisaccharides, oligosaccharides, polysaccharides, lipopolysaccharides, vitamins, steroids, hormones, cofactors, receptors, receptor ligands, and analogs and derivatives thereof. In some embodiments, the targeting agent is iRGD.

Also suitable for inclusion in the compositions described herein are programmable fusion lipids. Particles containing programmable fusion lipids have little tendency to fuse with cell membranes and deliver their payload until a given signal event occurs. This allows the composition to distribute more evenly after administration into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the particle membrane over time. By the time the particle is suitably distributed in the body, it has lost sufficient cloaking agent so as to be fusogenic. With other signal events, it is desirable to choose a signal that is associated with the disease site or target cell, such as lower pH at a site of tumor.

One or more complementary surface active agent can be added to the compositions, for example as complements to the characteristics of an amphiphilic agent or to improve particle stabilizing capacity or enable an improved solubilization. Such complementary agents can be pharmaceutically acceptable non-ionic surfactants which preferably are alkylene oxide derivatives of an organic compound which contains one or more hydroxylic groups. For example ethoxylated and/or propoxylated alcohol or ester compounds or mixtures thereof are commonly available and are well known as such complements to those skilled in the art. Examples of such compounds are esters of sorbitol and fatty acids, such as sorbitan monopalmitate or sorbitan monopalmitate, oily sucrose esters, polyoxyethylene sorbitane fatty acid esters, polyoxyethylene sorbitol fatty acid esters, polyoxyethylene fatty acid esters, polyoxyethylene alkyl ethers, polyoxyethylene sterol ethers, polyoxyethylene-polypropoxy alkyl ethers, block polymers and cethyl ether, as well as polyoxyethylene castor oil or hydrogenated castor oil derivatives and polyglycerine fatty acid esters. Suitable non-ionic surfactants, include, but are not limited to various grades of PLURONIC, POLOXAMER, SPAN, TWEEN, POLYSORBATE, TYLOXAPOL, EMULPHOR, or CREMOPHOR and the like. The complementary surface active agents can also be of an ionic nature, such as bile duct agents, cholic acid or deoxycholic their salts and derivatives or free fatty acids, such as oleic acid, linoleic acid and others. Other ionic surface active agents are found among cationic lipids like C₆-C₂₄ alkylamines or alkanolamine and cationic cholesterol esters.

In some embodiments, the composition comprises a PEG conjugated lipid and a phospholipid.

The composition can also include a targeting moiety, e.g., a targeting moiety that is specific to a cell type or tissue. The targeting moiety is also referred to as a targeting ligand or targeting agent herein. Targeting of particles with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013,556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fla. (1995). Other targeting moieties, such as ligands, cell surface receptors, glycoproteins, vitamins (e.g., riboflavin), aptamers and monoclonal antibodies, can also be used. The targeting moieties can include the entire protein or fragments thereof. Targeting mechanisms generally require that the targeting agents be positioned on the surface of the liposome in such a manner that the targeting moiety is available for interaction with the target, for example, a cell surface receptor.

In one approach, a targeting moiety, such as receptor binding ligand, can be linked to a component (e.g., a lipid) of the composition. In some embodiments, the ligand can be conjugated with PEG. A variety of different targeting agents and methods are known and available in the art, including those described, e.g., in Sapra, P. and Allen, T M, Prog. Lipid Res. 42(5):439-62 (2003); and Abra, R M et al., J. Liposome Res. 12:1-3, (2002). Other lipids conjugated with targeting moieties are described in US Patent Application Publication No. US2009/0247608 and No. US2012/0046478, content of both of which is incorporated herein by reference in its entirety.

Without limitation, a ligand can be selected from the group consisting of peptides, polypeptides, proteins, enzymes, peptidomimetics, glycoproteins, antibodies (monoclonal or polyclonal) and portions and fragments thereof, lectins, nucleosides, nucleotides, nucleoside and nucleotide analogues, nucleic acids, monosaccharides, disaccharides, trisaccharides, oligosaccharides, polysaccharides, lipopolysaccharides, vitamins, steroids, hormones, cofactors, receptors, receptor ligands, and analogs and derivatives thereof.

In some embodiments, the targeting ligand can be selected from the group consisting of polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, polyphosphazine, polyethylenimine, cspermine, spermidine, polyamine, pseudopeptide-polyamine, peptidomimetic polyamine, dendrimer polyamine, arginine, amidine, protamine, thyrotropin, melanotropin, lectin, surfactant protein A, mucin, transferrin, bisphosphonate, polyglutamate, polyaspartate, an aptamer, asialofetuin, hyaluronan, procollagen, insulin, transferrin, albumin, acridines, cross-psoralen, mitomycin C, TPPC4, texaphyrin, Sapphyrin, polycyclic aromatic hydrocarbons (e.g., phenazine, dihydrophenazine), bile acids, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, 03-(oleoyl)lithocholic acid, 03-(oleoyl)cholenic acid, dimethoxytrityl, or phenoxazine), RGD peptide, radiolabeled markers, haptens, naproxen, aspirin, dinitrophenyl, HRP, AP, lectins, vitamin A, vitamin E, vitamin K, vitamin B, folic acid, B12, riboflavin, biotin, pyridoxal, taxon, vincristine, vinblastine, cytochalasin, nocodazole, japlakinolide, latrunculin A, phalloidin, swinholide A, indanocine, myoservin, tumor necrosis factor alpha (TNFalpha), interleukin-1 beta, gamma interferon, GalNAc, galactose, mannose, mannose-6P, clusters of sugars such as GalNAc cluster, mannose cluster, galactose cluster, an aptamer, integrin receptor ligands, chemokine receptor ligands, serotonin receptor ligands, PSMA, endothelin, GCPII, somatostatin, cellular adhesion molecules (CAMS), and any combinations thereof.

A targeting agent can bind to and/or penetrate a specific cell type(s) at a greater rate than to other cell types, e.g. cancer cells as compared to healthy cells. A targeting agent can be selected from the group consisting of peptides, polypeptides, proteins, peptidomimetics, glycoproteins, lectins, nucleosides, nucleotides, nucleic acids, monosaccharides, disaccharides, trisaccharides, oligosaccharides, polysaccharides, lipopolysaccharides, vitamins, steroids, hormones, cofactors, receptors, receptor ligands, antibodies, antigen binding fragments of antibodies, and analogs and derivatives thereof. Targeting agents that preferentially bind to and/or cross the membrane of cancer cells are known in the art, e.g. iRGD, RGD, Lyp-1 peptide (CGNKRTRGC; SEQ ID NO:3), NGR peptide, iNGR, RGR peptide, CAR peptide, tCAR peptide (CARSKNK; SEQ ID NO: 2); FSH-33, Allatostatin 1, the pentapeptide CREKA, Hepatocarcinoma targeting peptide, Peptide GFE, anti-EGFR antibodies and/or antibody fragments, in particular Cetuximab, CendR, iRGD peptide (RGD-CendR hybrid peptide), small molecules, antibodies and/or antibody fragments binding to cancer-specific epitopes like e.g. CEA, Gastrin-releasing peptide receptors, Somatostatin receptors, Galanin receptors, Follicle-stimulating hormone receptors, p32 protein, Fibroblast growth factor receptors, HepG2, Epidermal growth factor receptors, Integrin .alpha.v.beta.6, Neuropilin-1 receptor and VEGF receptors and variants or combinations thereof. In some embodiments, a targeting agent can be iRGD, e.g. a peptide having the sequence CRGDKGPDC (SEQ ID NO: 1).

A targeting agent can be present, e.g. on the surface of a nanoparticle described herein and/or partially embedded in the membrane or lipid layer of a nanoparticle described herein. Methods of incorporating a targeting agent are known in the art and non-limiting examples are described elsewhere herein. In some embodiments, a composition described herein can comprise a two or more targeting agents, e.g. a composition can comprise a combination of nanoparticles, each comprising a different targeting agent and/or a composition can comprise nanoparticles which each comprise multiple targeting agents. In some embodiments, a composition described herein can comprise one targeting agent, two targeting agents, three targeting agents, or more targeting agents.

The composition comprising the conjugate can be in the form of a particle. Generally, the particle can be of any shape or form, e.g., spherical, rod, elliptical, cylindrical, capsule, or disc; and these particles can be part of a network or an aggregate. In some embodiments, the particle is a microparticle or a nanoparticle. As used herein, the term “microparticle” refers to a particle having a particle size of about 1 um to about 1000 um. As used herein, the term “nanoparticle” refers to particle having a particle size of about 0.1 nm to about 1000 nm. As used herein, the term “particle” encompasses liposomes, emulsions, vesicles and lipid particles. Without limitations, the particle can have any size from nm to millimeters.

Generally, the particles disclosed herein are nanoparticles and have an average diameter of from about 100 nm to about 500 nm. In some embodiments, the particles have an average diameter of from about 150 nm to about 400 nm, from about 200 nm to about 300 nm, from about 200 nm to about 250 nm, from about 75 nm to about 125 nm, from about 50 nm to about 500 nm, from about 75 nm to about 200 nm, from about 100 to about 175 nm, from about 125 nm to about 175 nm, from about 40 nm to about 90 nm, or from about 50 nm to about 80 nm.

In some embodiments a nanoparticle can be less than about 1 um in diameter, e.g., about 1 um or less in diameter, about 500 nm or less in diameter, about 400 nm or less in diameter, about 300 nm or less in diameter, about 200 nm or less in diameter, about 100 nm or less in diameter, about 50 nm or less in diameter, or about 10 nm or less in diameter. In some embodiments a nanoparticle can be less than 1 um in diameter, e.g., 1 um or less in diameter, 500 nm or less in diameter, 400 nm or less in diameter, 300 nm or less in diameter, 200 nm or less in diameter, 100 nm or less in diameter, 50 nm or less in diameter, or 10 nm or less in diameter. In some embodiments, the nanoparticles in a composition can be from about 1 nm to about 1 um in diameter, e.g. from about 1 nm to about 500 nm in diameter, from about 1 nm to about 200 nm in diameter, from about 10 nm to about 200 nm in diameter, from about 100 nm to about 200 nm in diameter, or from about 10 nm to about 100 nm in diameter. In some embodiments, the nanoparticles in a composition can be from 1 nm to 1 um in diameter, e.g. from 1 nm to 500 nm in diameter, from 1 nm to 200 nm in diameter, from 10 nm to 200 nm in diameter, from 100 nm to 200 nm in diameter, or from 10 nm to 100 nm in diameter.

In some embodiments, nanoparticles can be selected to be of specific sizes, e.g. is about (±10%) 225 nm in diameter. Methods of selecting nanoparticles of a particular size and/or range of sizes are known in the art and can include, by way of non-limiting example, filtration, sedimentation, centrifugation, and/or chromatographic methods, e.g. SEC.

It will be understood by one of ordinary skill in the art that particles usually exhibit a distribution of particle sizes around the indicated “size.” Unless otherwise stated, the term “particle size” as used herein refers to the mode of a size distribution of particles, i.e., the value that occurs most frequently in the size distribution. Methods for measuring the particle size are known to a skilled artisan, e.g., by dynamic light scattering (such as photocorrelation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

In some embodiments, the particles can be substantially spherical. What is meant by “substantially spherical” is that the ratio of the lengths of the longest to the shortest perpendicular axes of the particle cross section is less than or equal to about 1.5. Substantially spherical does not require a line of symmetry. Further, the particles can have surface texturing, such as lines or indentations or protuberances that are small in scale when compared to the overall size of the particle and still be substantially spherical. In some embodiments, the ratio of lengths between the longest and shortest axes of the particle is less than or equal to about 1.5, less than or equal to about 1.45, less than or equal to about 1.4, less than or equal to about 1.35, less than or equal to about 1.30, less than or equal to about 1.25, less than or equal to about 1.20, less than or equal to about 1.15 less than or equal to about 1.1. Without wishing to be bound by a theory, surface contact is minimized in particles that are substantially spherical, which minimizes the undesirable agglomeration of the particles upon storage. Many crystals or flakes have flat surfaces that can allow large surface contact areas where agglomeration can occur by ionic or non-ionic interactions. A sphere permits contact over a much smaller area.

The particles can be, e.g., monodisperse or polydisperse and the variation in diameter of the particles of a given dispersion can vary. In some embodiments, the particles have substantially the same particle size. Particles having a broad size distribution where there are both relatively big and small particles allow for the smaller particles to fill in the gaps between the larger particles, thereby creating new contact surfaces. A broad size distribution can result in larger spheres by creating many contact opportunities for binding agglomeration. The particles described herein are within a narrow size distribution, thereby minimizing opportunities for contact agglomeration. What is meant by a “narrow size distribution” is a particle size distribution that has a ratio of the volume diameter of the 90th percentile of the small spherical particles to the volume diameter of the 10th percentile less than or equal to 5. In some embodiments, the volume diameter of the 90th percentile of the small spherical particles to the volume diameter of the 10th percentile is less than or equal to 4.5, less than or equal to 4, less than or equal to 3.5, less than or equal to 3, less than or equal to 2.5, less than or equal to 2, less than or equal to 1.5, less than or equal to 1.45, less than or equal to 1.40, less than or equal to 1.35, less than or equal to 1.3, less than or equal to 1.25, less than or equal to 1.20, less than or equal to 1.15, or less than or equal to 1.1.

Geometric Standard Deviation (GSD) can also be used to indicate the narrow size distribution. GSD calculations involved determining the effective cutoff diameter (ECD) at the cumulative less than percentages of 15.9% and 84.1%. GSD is equal to the square root of the ratio of the ECD less than 84.17% to ECD less than 15.9%. The GSD has a narrow size distribution when GSD<2.5. In some embodiments, GSD is less than 2, less than 1.75, or less than 1.5. In one embodiment, GSD is less than 1.8.

In some embodiments, the composition is in the form of a liposome. As used herein, the term “liposome” encompasses any compartment enclosed by a lipid layer. Liposomes can have one or more lipid membranes. Liposomes can be characterized by membrane type and by size. Small unilamellar vesicles (SUVs) have a single membrane and typically range between 0.02 and 0.05·mu·m in diameter; large unilamellar vesicles (LUVS) are typically larger than 0.05·mu·m. Oligolamellar large vesicles and multilamellar vesicles have multiple, usually concentric, membrane layers and are typically larger than 0.1·mu·m. Liposomes with several nonconcentric membranes, i.e., several smaller vesicles contained within a larger vesicle, are termed multivesicular vesicles.

In order to form a liposome the lipid molecules comprise elongated non-polar (hydrophobic) portions and polar (hydrophilic) portions. The hydrophobic and hydrophilic portions of the molecule are preferably positioned at two ends of an elongated molecular structure. When such lipids are dispersed in water they spontaneously form bilayer membranes referred to as lamellae. The lamellae are composed of two mono layer sheets of lipid molecules with their non-polar (hydrophobic) surfaces facing each other and their polar (hydrophilic) surfaces facing the aqueous medium. The membranes formed by the lipids enclose a portion of the aqueous phase in a manner similar to that of a cell membrane enclosing the contents of a cell. Thus, the bilayer of a liposome has similarities to a cell membrane without the protein components present in a cell membrane.

A liposome composition can be prepared by a variety of methods that are known in the art. See e.g., U.S. Pat. Nos. 4,235,871, 4,897,355 and 5,171,678; published PCT applications WO 96/14057 and WO 96/37194; Feigner, P. L. et al., Proc. Natl. Acad. Sci., USA (1987) 8:7413-7417, Bangham, et al. M. Mol. Biol. (1965) 23:238, Olson, et al. Biochim. Biophys. Acta (1979) 557:9, Szoka, et al. Proc. Natl. Acad. Sci. (1978) 75: 4194, Mayhew, et al. Biochim. Biophys. Acta (1984) 775:169, Kim, et al. Biochim. Biophys. Acta (1983) 728:339, and Fukunaga, et al. Endocrinol. (1984) 115:757, content of all of which is incorporated herein by reference in its entirety.

The liposomes can be prepared to have substantially homogeneous sizes in a selected size range. One effective sizing method involves extruding an aqueous suspension of the liposomes through a series of polycarbonate membranes having a selected uniform pore size; the pore size of the membrane will correspond roughly with the largest sizes of liposomes produced by extrusion through that membrane. See e.g., U.S. Pat. No. 4,737,323, content of which is incorporated herein by reference in its entirety.

The compositions described herein can also be in the form of an emulsion. Emulsions are typically heterogenous systems of one liquid dispersed in another in the form of droplets (Idson, in Pharmaceutical Dosage Forms, Lieberman, Rieger and Banker (Eds.), 1988, Marcel Dekker, Inc., New York, N.Y, volume 1, p. 199; Rosoff, in Pharmaceutical Dosage Forms, Lieberman, Rieger and Banker (Eds.), 1988, Marcel Dekker, Inc., New York, N.Y, Volume 1, p. 245; Block in Pharmaceutical Dosage Forms, Lieberman, Rieger and Banker (Eds.), 1988, Marcel Dekker, Inc., New York, N.Y, volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the conjugate disclosed herein can be present as a solution in either the aqueous phase or the oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers can broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y, volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y, volume 1, p. 285; Idson, in Pharmaceutical Dosage Forms, Lieberman, Rieger and Banker (Eds.), Marcel Dekker, Inc., New York, N.Y, 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y, volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials can also be included in emulsion formulations and contribute to the properties of emulsions. These include, but are not limited to, fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y, volume 1, p. 335; Idson, in Pharmaceutical Dosage Forms, Lieberman, Rieger and Banker (Eds.), 1988, Marcel Dekker, Inc., New York, N.Y, volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The applications of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y, volume 1, p. 245; Idson, in Pharmaceutical Dosage Forms, Lieberman, Rieger and Banker (Eds.), 1988, Marcel Dekker, Inc., New York, N.Y, volume 1, p. 199).

The compositions can include one, two, or more different conjugates as described herein. In some embodiments, the composition further comprises an anticancer agent in addition to the conjugate. In some embodiments, the anticancer agent is a platinum compound, paclitaxel; carboplatin; bortezomib; vorinostat; rituximab; temozolomide; rapamycin; an alkylating agent; cyclosphosphamide; an alkyl sulfonate; busulfan; improsulfan; piposulfan; an aziridine; an ethylenimine; a methylamelamine; an acetogenin; a camptothecin; a cryptophycin; a nitrogen mustard; a nitrosurea; an antibiotic; a enediyne antibiotic; a bisphosphonate; doxorubicin; a mitomycin; an anti-metabolite; a folic acid analogue; a purine analog; a pyrimidine analog; an androgen; an anti-adrenal; an epothilone; a maytansinoid; a trichothecene; gemcitabine; 6-thioguanine; mercaptopurine; methotrexate; vinblastine; etoposide; ifosfamide; mitoxantrone; vincristine; vinorelbine; novantrone; teniposide; edatrexate; daunomycin; aminopterin; xeloda; ibandronate; irinotecan; a topoisomerase inhibitor; a retinoid; capecitabine; combretastatin; leucovorin; lapatinib; and erlotinib.

Chemotherapeutic Agents

In some embodiments, no active compounds other than the HSP90 inhibitor are included in the compositions. Tn some embodiments, other active compositions are included; for example, the compositions can also include chemotherapeutic agents. As used herein, the term “chemotherapeutic agent” refers to any chemical or biological agent with therapeutic usefulness in the treatment of diseases characterized by abnormal cell growth. Such diseases include tumors, neoplasms and cancer as well as diseases characterized by hyperplastic growth. These agents can function to inhibit a cellular activity upon which the cancer cell depends for continued proliferation. In some aspect of all the embodiments, a chemotherapeutic agent is a cell cycle inhibitor or a cell division inhibitor. Categories of chemotherapeutic agents that are useful in the methods of the invention include alkylating/alkaloid agents, antimetabolites, hormones or hormone analogs, and miscellaneous antineoplastic drugs. Most of these agents are directly or indirectly toxic to cancer cells. In one embodiment, a chemotherapeutic agent is a radioactive molecule. One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed. 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). In some embodiments, the chemotherapeutic agent can be a cytotoxic chemotherapeutic. The term “cytotoxic agent” as used herein refers to a substance that inhibits or prevents the function of cells and/or causes destruction of cells. The term is intended to include radioactive isotopes (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents, and toxins, such as small molecule toxins or enzymatically active toxins of bacterial, fungal, plant or animal origin, including fragments and/or variants thereof.

The term chemotherapeutic agent is a broad one covering many chemotherapeutic agents having different mechanisms of action. Generally, chemotherapeutic agents are classified according to the mechanism of action. Many of the available agents are antimetabolites of development pathways of various tumors, or react with the DNA of the tumor cells. There are also agents which inhibit enzymes, such as topoisomerase I and topoisomerase II, or which are antimiotic agents.

Chemotherapeutic agents include, but are not limited to, an aromatase inhibitor; an antiestrogen, an anti-androgen (especially in the case of prostate cancer) or a gonadorelin agonist; a topoisomerase I inhibitor or a topoisomerase II inhibitor; a microtubule active agent, an alkylating agent, an anti-neoplastic anti-metabolite or a platin compound; a compound targeting/decreasing a protein or lipid kinase activity or a protein or lipid phosphatase activity, a further anti-angiogenic compound or a compound which induces cell differentiation processes; a bradykinin 1 receptor or an angiotensin II antagonist; a cyclooxygenase inhibitor, a bisphosphonate, a heparanase inhibitor (prevents heparan sulphate degradation), e.g., PI-88, a biological response modifier, preferably a lymphokine or interferons, e.g. interferon .gamma., an ubiquitination inhibitor or an inhibitor which blocks anti-apoptotic pathways; an inhibitor of Ras oncogenic isoforms or a farnesyl transferase inhibitor; a telomerase inhibitor, e.g., telomestatin; a protease inhibitor, a matrix metalloproteinase inhibitor, a methionine aminopeptidase inhibitor, e.g., bengamide or a derivative thereof; a proteasome inhibitor, e.g., PS-341 (bortezomib/Velcade); agents used in the treatment of hematologic malignancies or FMS-like tyrosine kinase inhibitors; an HSP90 inhibitors; histone deacetylase (HDAC) inhibitors; mTOR inhibitors; somatostatin receptor antagonists; integrin antagonists; anti-leukemic compounds; tumor cell damaging approaches, such as ionizing radiation; EDG binders; anthranilic acid amide class of kinase inhibitors; ribonucleotide reductase inhibitors; S-adenosylmethionine decarboxylase inhibitors; antibodies against VEGF or VEGFR; photodynamic therapy; angiostatic steroids; ATI receptor antagonists; ACE inhibitors; and the like.

Other chemotherapeutic agents include, but are not limited to, plant alkaloids, hormonal agents and antagonists, biological response modifiers, preferably lymphokines or interferons, antisense oligonucleotides or oligonucleotide derivatives; or miscellaneous agents or agents with other or unknown mechanism of action.

In some embodiments, the chemotherapeutic agent is a taxane. The term “Taxane” generally refers to diterpene-containing compounds produced by the plants of the genus Taxus (e.g., yews, such as, but not limited to, Taxus baccata, Taxus brevifolia, Taxus canadensis, Taxus chinensis, Taxus cuspidata, Taxus floridana, Taxus globosa, Taxus sumatrana, Taxus walUchiana), and synthetic and semisynthetic forms thereof. See, e.g., U.S. Pat. No. 9,789,193. The basic taxane core structure may further be substituted or may contain unsaturations in the ring to yield a number of compounds, generically known as taxanes. Generally, such compounds may block cell growth by stopping mitosis by interfering with microtubules. The term “diterpene,” as used herein, means chemical compounds having a carbon skeleton derived from four isoprene units. The taxane group of compounds includes paclitaxel and docetaxel.

Taxanes can be isolated from natural sources, and can also be prepared synthetically from naturally occumng precursors. Paclitaxel (TAXOL, Bnstol-Myers Squibb), for example, can be prepared from baccatin by attachment of protecting groups to the hydroxyl groups of baccatin that are to become the hydroxyl groups of paclitaxel, converting the precursor baccatin to paclitaxel, and then removing the protecting groups from the hydroxyl groups to obtain paclitaxel (see, e.g., WO93/10076; K. V Rao, U.S. Pat. No. 5,200,534; R. A. Holton, U.S. Pat. No. 5,015,744; PCT US92/07990; V. J. Stella and A. E. Mathew, U.S. Pat. No. 4,960,790; K. C. Nicolau, Nature 3j54 (1993), pp. 464-466; Nicolau, K. C. et al. Nature 367 (1994) pp. 630-634; Holton, R. A., et al. J. Am. Chem. Soc. H6 (1994) pp. 1597-1600; WO93/16059, int. pub. date Aug. 19, 1993; EP 528.729, published Feb. 24, 1993; EP 522,958, published Jan. 13, 1993; WO91/13053, int. pub. date Sep. 5, 1991; EP 414,610, int. pub. date Feb. 27, 1991; the contents of these documents are incorporated herein by reference). Non-limiting examples of taxanes can include paclitaxel and docetaxel, derivatives thereof, and mixtures thereof.

Taxanes can be used effectively to treat a variety of cancers. Paclitaxel, for example, has been found to have activity against ovatan and breast cancers, as well as against malignant melanoma, colon cancer, leukemias and lung cancer (see, e.g., Borman, Chemical & Engineering News, Sep. 2, 1991, pp. 11-18; The Pharmacological Basis of Therapeutics (Goodman Gilman et al., eds.), Pergamon Press, New York (1990), p. 1239; Suffness, Antrtumor Alkaloids, in: “The Alkaloids, Vol. XXV,” Academic Press, Inc. (1985), Chapter 1, pp. 6-18; Rizzo et al., J. Pharm. & Biomed. Anal. .sctn.(2):159-164 (1990); and Biotechnology 9:933-938 (October. 1991). Paclitaxel acts against cancer cells by binding to tubulin in the cells nuclei, thereby blocking the disassembly of microtubules and consequently, inhibiting cell division (Schiff et al., Nature 277:665 (1979). In one embodiment, the taxane is paclitaxel.

One exemplary composition comprises or consists of a taxane and a radicicol-cholesterol conjugate intercalated, entrapped, or confined in a lipid bilayer, e.g., inside of, in the membrane of, or on the surface of a nanoparticle. In this composition as well as in some of the other compositions described herein, the taxane rapidly releases into the cells that uptake the composition prior to the Hsp90 inhibitor, and the Hsp90 inhibitor releases more slowly into the cells that uptake the composition thereafter. In addition, differing amounts of the two are preferably provided. For example, in the taxane radicicol composition, a larger amount of radicicol can be included in the NP.

The use of a taxane, for example docetaxel, in the NP achieves a couple of goals. First, the docetaxel is ‘encapsulated’ in the Hsp90 nanoparticle at a defined ratio (in the example(s) the Docetaxel, the Radicicol-cholesterol conjugate, the L-α-phosphatidylcholine, and the DSPE-PEG2000 are included at 0.01:0.09:0.6:0.3 molar ratios) and it is shown that the release of the docetaxel from the NP was faster than the release of the Hsp90, which creates an environment of cancer cells susceptible to Hsp90 inhibition and therefore cell death. The results herein demonstrate that docetaxel should be released first to induce a phenotype in cancer cells that creates susceptibility to Hsp90 inhibition.

Methods of Treatment

The methods described herein include methods for the treatment of disorders associated with abnormal apoptotic or differentiative processes, e.g., cellular proliferative disorders or cellular differentiative disorders, e.g., cancer, including both solid tumors and hematopoietic cancers. In some embodiments, the disorder is a solid tumor, e.g., breast, prostate, pancreatic, brain, hepatic, lung, kidney, skin, or colon cancer. Generally, the methods include administering a therapeutically effective amount of a treatment comprising a composition or conjugate as described herein, to a subject who is in need of, or who has been determined to be in need of, such treatment. The present methods can be used, e.g., in mammalian subjects, e.g., human or non-human veterinary subjects (e.g., non-human primate, mouse, rat, dog, cat, horse, or cow).

As used in this context, to “treat” means to ameliorate at least one symptom of the disorder associated with abnormal apoptotic or differentiative processes. For example, a treatment can result in a reduction in tumor size or growth rate. Administration of a therapeutically effective amount of a compound described herein for the treatment of a condition associated with abnormal apoptotic or differentiative processes will result in a reduction in tumor size or decreased growth rate, a reduction in risk or frequency of reoccurrence, a delay in reoccurrence, a reduction in metastasis, increased survival, and/or decreased morbidity and mortality, inter alia.

Examples of cellular proliferative and/or differentiative disorders include cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and liver origin.

As used herein, the terms “cancer”, “hyperproliferative” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as affecting lung, breast, thyroid, lymphoid, gastrointestinal, and genitourinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the cancer is triple negative breast cancer.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Preferably, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Sternberg disease.

Described herein are methods of using the compositions to treat patients having a drug-resistant cancer, or cells that have become drug resistant, e.g., TNBC that has become resistant to treatment with taxanes and/or anthracyclines. Also provided herein are methods for increasing the number of NKG2D ligand receptors on tumor cells comprising treating the tumor cells with a composition of the invention, thereby attracting and activating endogenous and adoptive NK cells thereby.

In some embodiments, the methods include administering an NK cell-based cancer immunotherapy, e.g., adoptive NK cell transfer, natural killer cells (NK cells, i.e., CD3⁻ cells), e.g., derived from healthy donor derived peripheral blood, induced pluripotent stem cells (iPSC), umbilical cord stem cells, oNKord cells (allogeneic partial HLA-matched NK cells derived from UCB-CD34+ progenitors), placenta-expanded NK cells (CYNK-001), CTV-1 lysate-primed human NK cells (CNDO-109-NK cells), or other natural sources; or NK-92 cells, NK-101 cells, or other NK cells obtained and expanded from patients with NK lymphomas, or variants of each of these, which can be genetically modified by chimeric antigen receptors (e.g., CAR-NK cells), Bi- and tri-specific killer engagers, BiKEs and TriKEs (e.g., CD16/IL-15/CD33 TriKE, GTB-3550), Tri-functional NK cell engagers (NKCEs) that crosslink both NKp46 and CD16; see, e.g., Liu et al., Journal of Hematology & Oncology 14, Article number: 7 (2021). Preferably, the methods include administering a composition, e.g., a nanoparticle, as described herein, and waiting until the composition is cleared from the systemic circulation before treating with adoptive NK cells (see FIGS. 3N and 3O).

The present methods can includes administering a composition as described herein to the tumor cells, e.g., to activate the NK cells.

The present methods can be used for increasing sensitivity of drug resistant cancer cells to kinase inhibitors or other cancer chemotherapies comprising pre-treating the cancers cells with a composition as described herein. In some embodiments, the methods include administering a chemotherapeutic agent, e.g., a taxane, e.g., docetaxel, before administering a conjugate as described herein.

Preferably, the methods include administering a conjugate as described herein and a chemotherapeutic agent together or substantially simultaneously (e.g., within 2 hours, 1 hour, 30 minutes, 20 minutes, 10 minutes, or 5 minutes of each other). Preferably the present conjugates provide slow release of the Hsp90 inhibitor from the nanoparticle/conjugate. Without wishing to be bound by theory, it is believed that this has the effect of increasing the immunogenic phenotype of cancer cells and targeting drug-induced resistance conferred by the chemotherapeutic.

In some embodiments, a composition described herein comprising a lipid-HSP90i conjugate is administered in combination with standard of care chemotherapy for a cycle or two or three. After allowing time for the pharmacokinetic clearance of the nanoparticle, an NK cell-based cancer immunotherapy is then administered.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity a composition as described herein. The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition as described herein can be administered over a period of time, such as over a 5 minute, 10 minute, 15 minute, 20 minute, or 25 minute period.

Pharmaceutical Compositions and Methods of Administration

The methods described herein include the use of pharmaceutical compositions comprising or consisting of the conjugates described herein as an active ingredient.

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., chemotherapeutics or other anti-cancer agents.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Pharmaceutical compositions, dosage forms, dosage regimens, adjuvants, and effective amounts can be extrapolated from the data in the detailed description or from the disclosures in the patent publications cited herein and incorporated by reference.

Other lipid-conjugate formulations that may be employed in the invention include those set forth in U.S. Pat. No. 10,730,899 and 10,426,753. However, not all lipid conjugate formulations are alike.

### EXAMPLES

The invention is further described in the following examples, which do not limit the scope of the invention described in the claims.

### Example 1. Nano-Engineered Disruption of Heat Shock Protein 90 (Hsp90) Targets Drug-Induced Resistance and Relieves Natural Killer Cell Suppression in Breast Cancer

Materials and Methods

The following materials and methods were used in Example 1.

Animal Welfare and Human Samples

All in vivo experiments were performed in compliance with active IACUC protocol approved through Harvard Medical School and Brigham and Women's Hospital, and in accordance with institutional guidelines, supervised on-site by veterinary staff. Mice with tumors were closely monitored by careful clinical examination to detect deterioration of their physical condition and sacrificed at any sign of stress. Human samples for ex vivo experiments were obtained from patients clinically diagnosed with TNBC and were collected by Mitra Biotech under institutional review board (IRB) approval with written informed consent from each patient.

Materials

Radicicol was a kind gift from Dr. Leslie Gunatilaka (University of Arizona). All chemical reagents were of analytical grade, used as supplied without further purification and purchased from Signal-Aldrich, unless indicated. Recombinant human cytokines were reconstituted in a solution containing 0.1 mM acetic acid and 0.1% BSA (Peprotech).

Cell Culture and Generation of Drug Tolerant Cancer Cells (DTCCs)

Human MDA-MB-231, MDA-MB-468, MDA-MB-436, MCF-7, mammary carcinoma 4T-1 cells (ATCC) and SUM159 (Bioivt) were purchased in the last 10 years and cultured in 10% fetal bovine serum in DMEM or RPMI media (Invitrogen, Carlsbad CA, USA). Cell lines were validated for absence of mycoplasma prior to use, by the sourcing agency. Cells were used within 10 passages from frozen stock vials obtained from the sourcing agency. NK-92MI (ATCC) were cultured according to manufacturer protocol. Primary human peripheral blood CD56+ natural killer cells (Stem Cell Technologies, catalog #70037) were cultured using Immunocult XF T-cell expansion media (Stem Cell Technologies) with 10% FBS and 100 U/ml human recombinant IL-2. 3-dimensional tumor spheroid NanoCulture plates were used whenever indicated (MBLI, Woburn, MA).

Gene Knockdown

siRNA gene knockdown was performed on cells at a concentration of 5×10⁴ cells ml⁻¹. Pre-validated Silencer Select siRNA targeting (sense sequences) MICA (siRNAs ID #1: s8772, ID #2: s458040; Thermo Fisher Inc., Rockford, IL, USA), and were transfected using lipofectamine 2000 (Invitrogen, Grand Island, NY, USA) following manufacturer's protocol. Scrambled siRNA was used as a control.

Cell Viability Assays

Cell viability was measured as described using 3-(4,5-dimethylthiazol-2-yl)-5-(3-carboxymethoxyphenyl)-2-(4-sulfophenyl)-2H-tetrazolium (MTS; reagent, Promega, Madison, WI, USA) or water-soluble tetrazolium salts (WST reagent; Dojindo Molecular Technologies Inc., Rockville, MD, USA) following manufacturer's protocol and absorbance was read at the recommended UV wavelength (450 nm) using BioTek microplate reader (BioTek Instruments Inc., Winooski, VT, USA). To evaluate the pharmacological interaction of different combinations of drugs, we followed the method proposed by Chou et al. (23).

Phosphorylation Arrays

The Proteome Profiler Human Phospho Array (R&D systems, Minneapolis MN, USA) was used to identify phosphorylated residues affiliated with different proteins. Following the Bradford protein analysis assay to normalize total protein content, cell lysate from the indicated cell line was applied to the phosphorylation membranes following manufacturer's protocol. Optical densities were determined by Image J software (NIH.gov) and Adobe CS5. Reference spots were used to normalize between array membranes.

Immunoblotting

Protein samples were resolved by SDS-PAGE and transferred to PVDF membranes prior to incubation at 4° C. with indicated primary antibodies, mTOR and pMTOR^{Ser2448}, pAKT^{Thr308} and AKT, Phospho-p44/42 MAPK (Erk1/2)^{Thr202/Tyr204}, p44/42 MAPK (Erk1/2) pPRAS40^{Thr246} pSTAT3^{Tyr705}, STAT3, PRAS40 and β-Actin antibodies were purchased from Cell Signaling Technology, pHck^{Tyr410} (Thermo Fisher Scientific) MICA/B (R&D Systems, Minneapolis, MN) and HSF-1 and HSF-1^{Ser326} (abcam). Western blotting images chosen as representative depictions in the figures demonstrate equivalent results taken from biological replicates (N≥3).

Flow Cytometry

Cells were cultured as indicated, fixed with 4% paraformaldehyde, washed twice with PBS and blocked in 10% goat serum (v/v). Cells were incubated with fluorescently labeled antibodies for NKG2D, CD158a, NKB1, CD244, KLRG1 (BioLegend, San Diego, CA), or MICA/B (R&D Systems, Minneapolis, MN) overnight at 4° C. and analyzed (C6 Accuri cyomteters Inc. Ann Arbor, MI). Data analysis was performed using FlowJo software (Tree Star Inc., Ashland OR) and Accuri cFlow plus software to obtain and confirm mean fluorescent intensity (GNU.org). Isotype IgG control was used to subtract for background noise.

Ex Vivo Human Tumor Experiments

Human TNBC was collected immediately after surgical resection (See supplemental Table 2 for metadata). Matched-patient non-heparinized blood (5-10 mL) was also collected at the time of biopsy in BD-Vacutainer tubes (Becton Dickinson) following published protocol and established quality control criteria (24). Tissue slices were maintained in customized tumor matrix protein (TMP) coated plates as described in prior report (25). Tissue fragments (approximately 300 μm-2 mm in size) were treated with the indicated drugs at the clinical max concentration (Cmax) for 72 hours as determined by published literature on each drug pharmacokinetic profile (See supplemental Table 3 for related drug concentrations used). DMSO was used as a vehicle control.

Cytokine Analysis

Media was collected from parental cell lines or DTCCs cultured for indicated time points, centrifuged (‘neat’) and the resulting supernatant was aliquoted and stored at −80° C. Using thawed conditioned media (25 μL ‘neat’ media), a panel of 41 cytokines and chemokines were profiled using the MILLIPLEX MAP Human Cytokine/Chemokine Magnetic Bead Panel (MilliporeSigma, Burlington, MA, USA) according to the manufacturer's instructions and plates were read on the Luminex200 (Luminex Corp., Austin, TX, USA). Analyte measurements were reported using the MILLIPLEX analyst software (MilliporeSigma, Burlington, MA, USA).

In Vivo Experiments

4T1 mouse mammary carcinoma cells (1×10⁶ cells) were injected into the mammary fat pads of 5-6-week-old female balb/c mice (BALB/cAnNCrl, Charles River, Strain Code: 028). Docetaxel (DTX) was dissolved in pure ethanol at a concentration of 50 mg/mL mixed 1:1 with polysorbate 80 (Tween 80) and brought to a final working concentration with 5% glucose in PBS. Once tumors became palpable (˜100 mm³), docetaxel, radicicol, DocRad-NP or vehicle treatments were administered intravenously (i.v.). on indicated days at the indicated doses. Tumor volumes were quantified using digital calipers (Starlett, Athol, MA) by a third party unaware of treatment conditions.

Immunofluorescence and Confocal Microscopy

Cells were permeabilized by incubation with 0.5% Triton X-100 at 4° C. for 10 min, washed three times with 1×PBS-T (1× PBS+0.05% Tween 20) and blocked using 10% BSA solution (dilution with PBS-T) at room temperature for 1 hour. The samples were stained with a primary antibodies: Hsp90 (Cell signaling technology), phosphorylated HSF-1^{Ser326} (Novus Biological) and phosphorylated Hck^{Ty410} (Thermofisher Scientific). For nuclear staining, the samples were counter stained with 4′,6-diamidino-2-phenylindole (DAPI; Thermo Fisher Scientific). Images were taken on a Nikon Eclipse Ti camera (Nikon Instruments) with NIS Elements Imaging Software (3.10). Confocal fluorescence imaging was performed on Zeiss LSM 800, Airyscan Confocal Laser Scanner Microscope with Zen 2.3 software. Post processing of the images was completed either in Image J or Zen lite software.

Immunohistochemistry (IHC) and Multiplex IHC (mIHC) Analysis

For murine tissue IHC, FFPE sections were incubated with the following primary antibodies; phosphorylated PRAS40^{Thr246} (clone C77D7), STAT3^{Tyr705} (D3A7) (Cell Signaling Technology, Danvers MA), CD49b (PA5-87012, ThermoFisher), MULT-1 (ABIN966609, antibodies online) and Rae-1 (PA5-93166, Invitrogen). Sections were then incubated with a HRP-conjugated secondary antibody (SignalStain® Boost IHC Detection Reagent; Cell Signaling Technology). Chromogenic development of signal was performed using 3,3′-diaminobenzidine (DAB Peroxidase Substrate Kit; Vector Laboratories). Terminal deoxynucleotidyl transferase dUTP nick end labeling (TUNEL) was used following manufacturer protocol (FITC kit, Genscript, Piscataway NJ). For ex vivo human tumor experiments, tissue was prepared from FFPE in serial 4 μm sections and cut onto charged slides, which were stained with hematoxylin and eosin (H&E) for pathological determination of tumor viability and area (determined by a clinical pathologist), cleaved caspase-3 in vitro diagnostic (IVD) antibody (Cat #229, Biocare) or stained with a 4-plex panel of fluorophore dyes (Opal DAPI (Cat #FP1490), Discovery FAM (Cat #760-243, green), Discovery Cy5 (Cat #760-238)) with corresponding primary marker antibodies (FAM-CD56 IVD antibody (Clone #MRQ-420, Ventana, Cat #790-4596), Anti-CD3 IVD antibody (Clone #2GV6, rabbit monoclonal, Ventana, Cat #790-4341), anti-pan keratin (PanCK; clone AE1/AE3/PCK26, Ventana, Cat #760-2595)) selected for profiling natural killer cells (DAPI⁺PanCK⁻CD56⁺CD3⁻).

Multiplex IHC (mIHC) Image Analysis

H&E stains were annotated digitally by a clinical pathologist (David Goldman, MD, co-author) to designate tumor tissue, non-tumor tissue and stromal areas using the HALO™ digital image analysis software version 2.3.1.2089.70 (Indica Labs, Corrales, NM, USA) to establish tumor, non-tumor and stroma ROI (regions of interest). ROI groups were then trained based on ‘ground truth’ and cell populations were segmented and optimized using the DAPI stain. Once all algorithms had been fully developed, there were bulk applied to the appropriate patient, establishing a data set identifying the absolute count and spatial distribution of DAPI⁺PanCK⁻CD56⁺CD3⁻ cells in tumor, non-tumor and stromal ROI. HALO Spatial analysis (Indica Labs, Corrales, NM, USA) module was used for plotting the NK data set containing the requisite X and Y coordinate map. Computer software settings and details are provided in the supplemental information file. For spearman correlation analysis, data were normalized and read into the R statistical computing package “car”. Data tables were created caspase 3 high and caspase 3 low samples (respectively). These data were fed into a variety of visualization packages (GGPlot, GGPairs, scatterplotMatrix, and corr). For Correlation analysis—The R corrplot package was utilized to visually interpret the output from the above analysis. Spearman output was visualized by creating a heatmap to express the individual correlation values that were observed.

### Synthesis of Radicicol-Cholesterol Conjugate

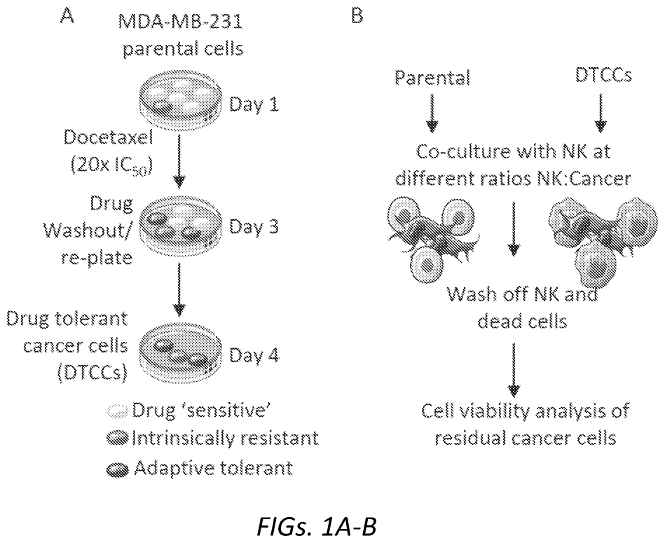

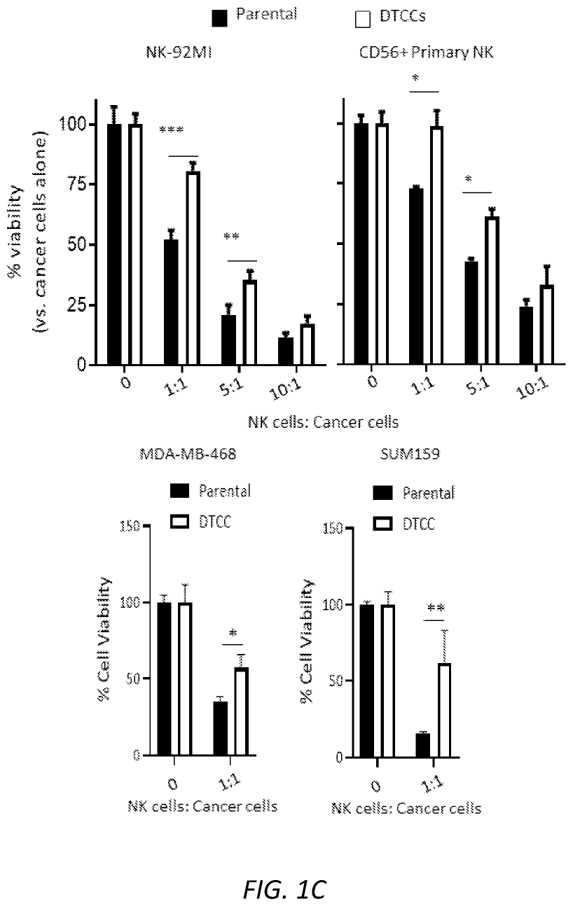

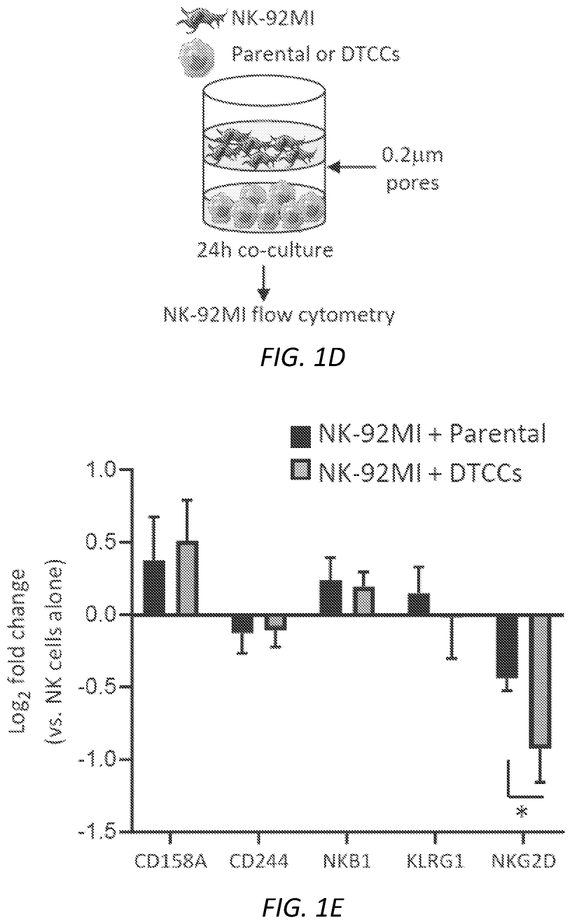

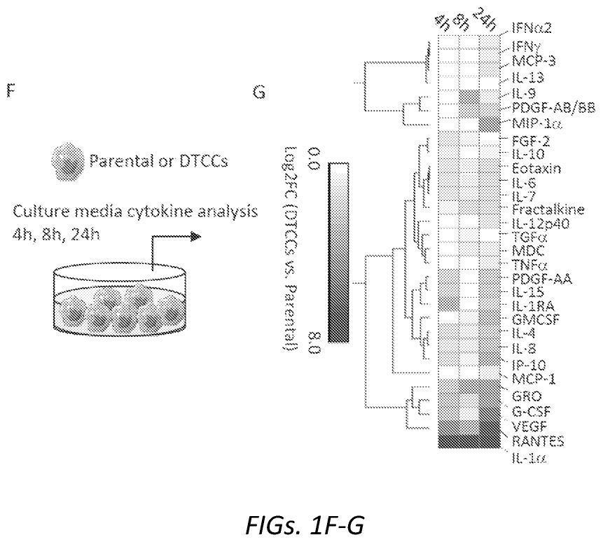

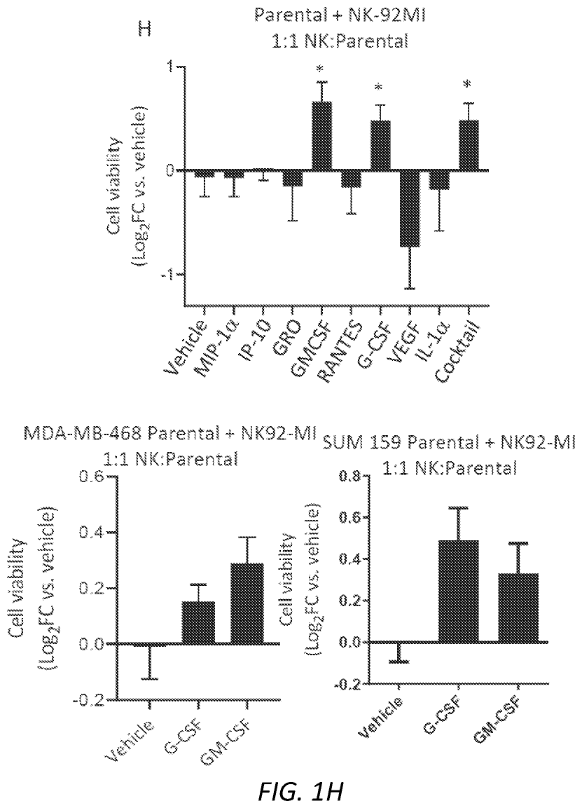

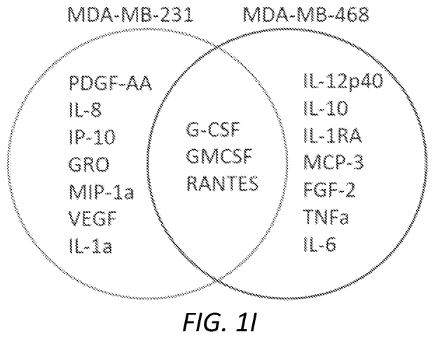

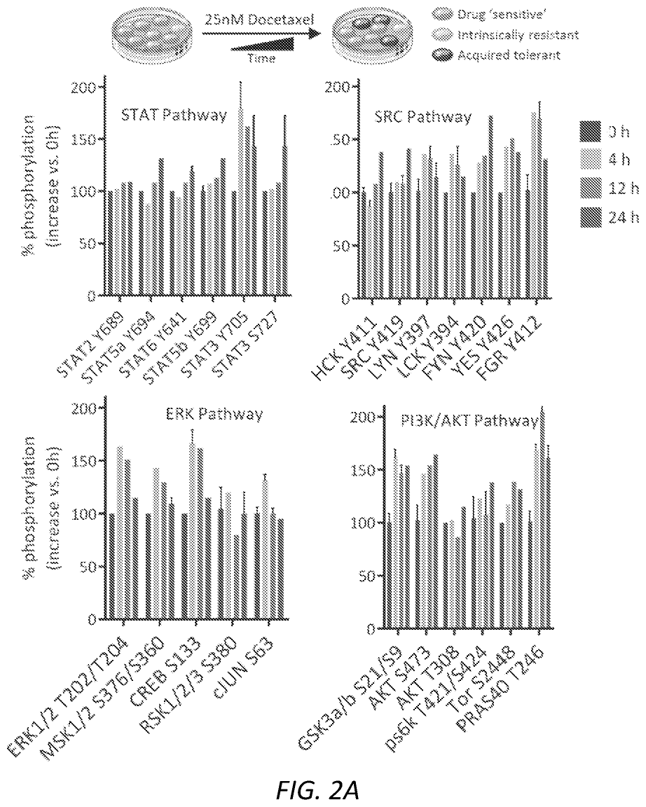

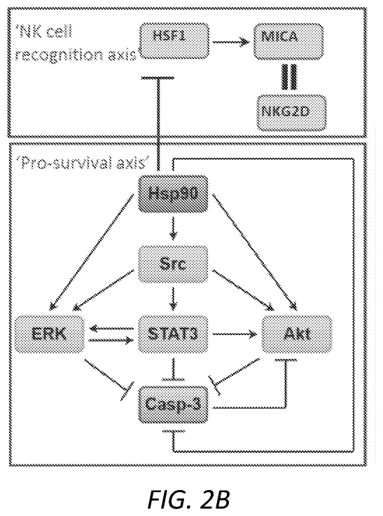

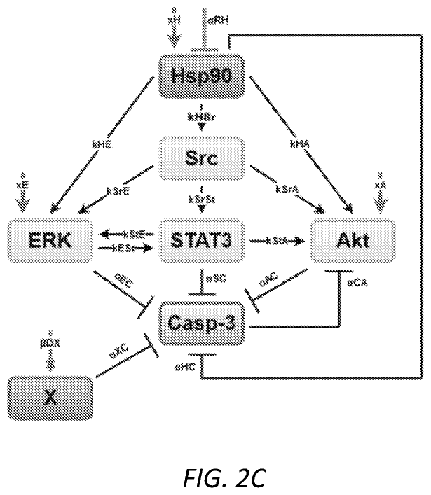

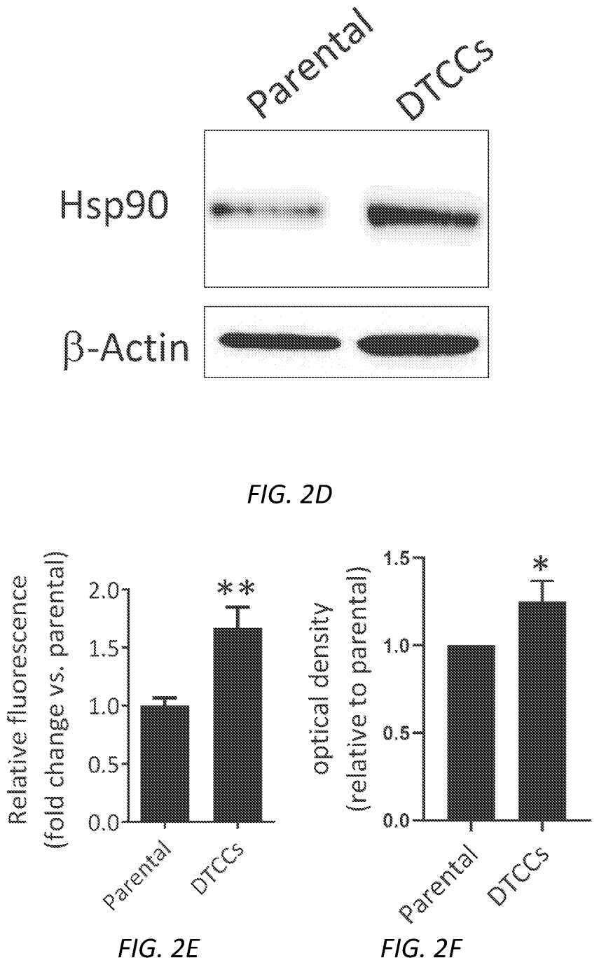

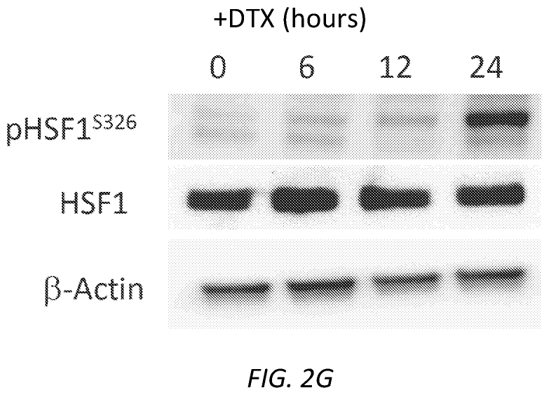

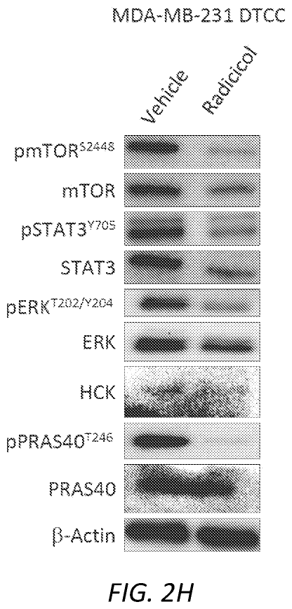

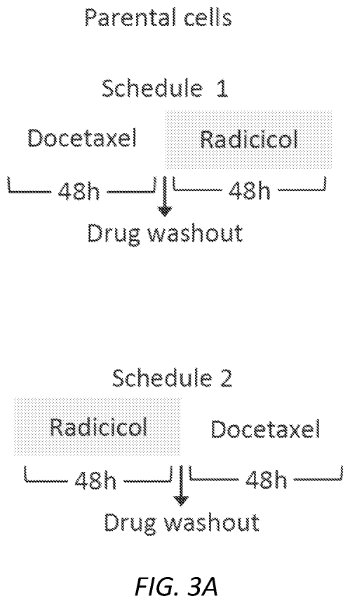

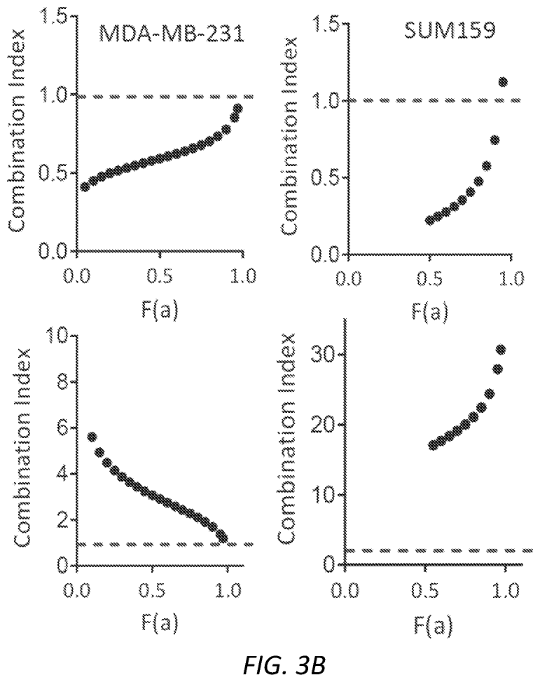

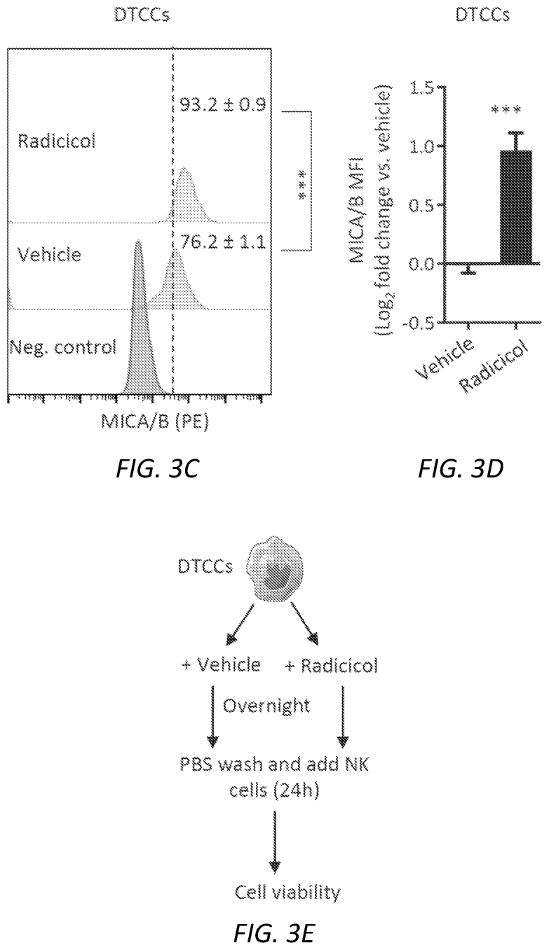

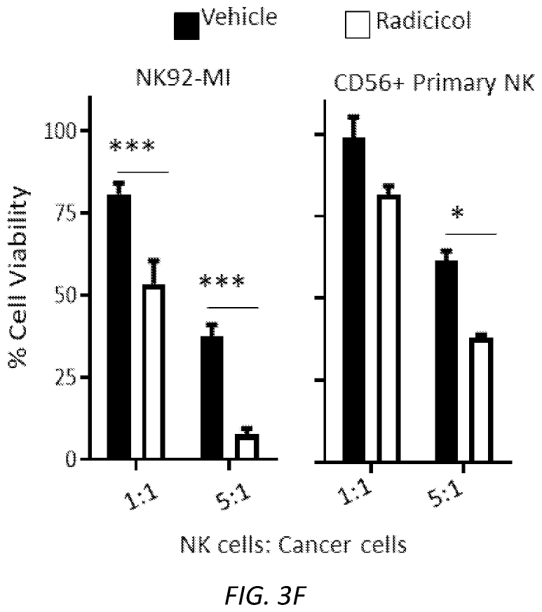

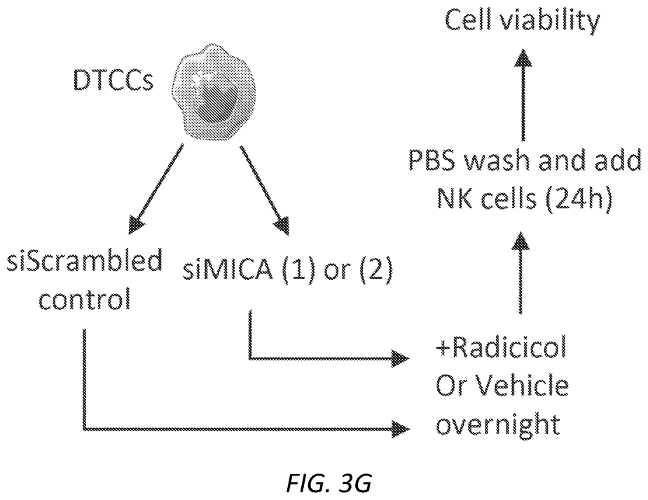

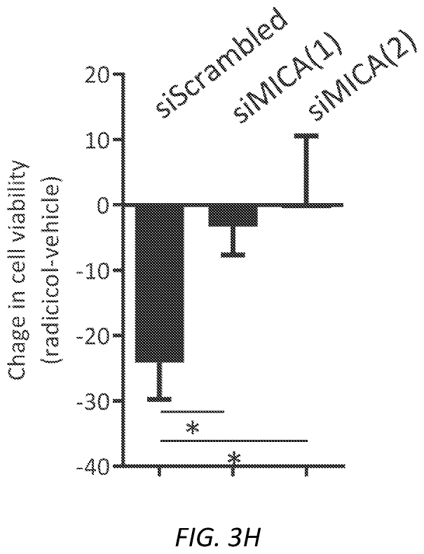

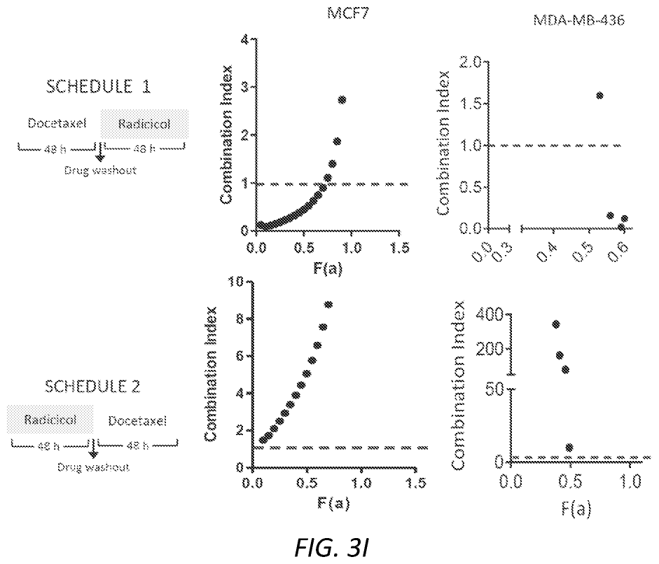

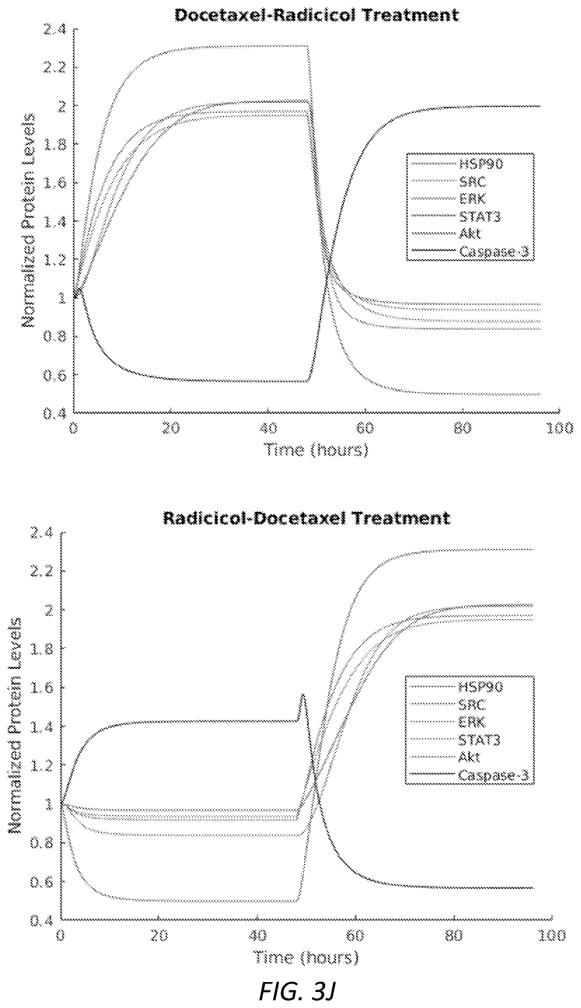

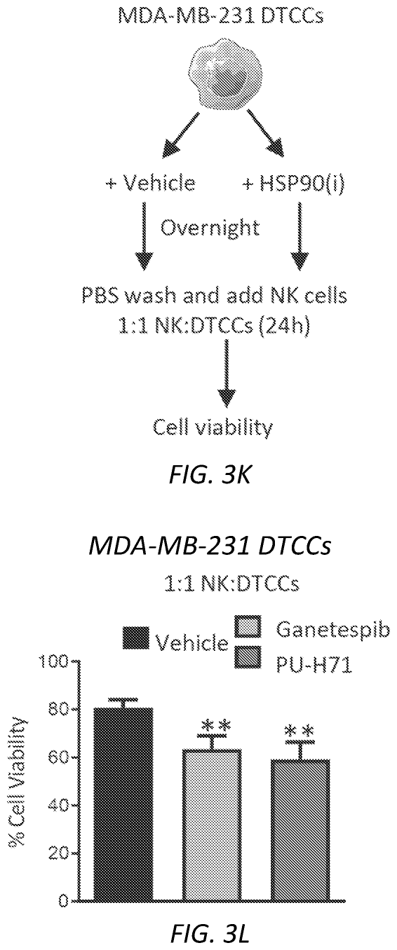

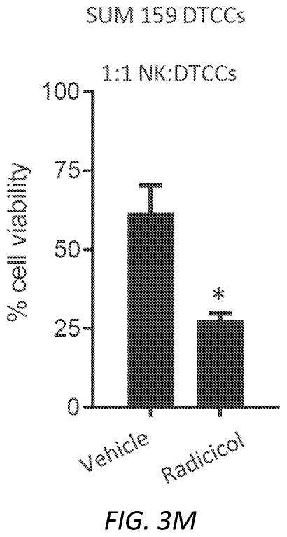

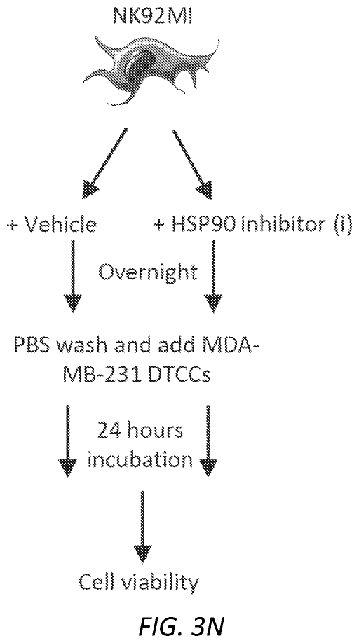

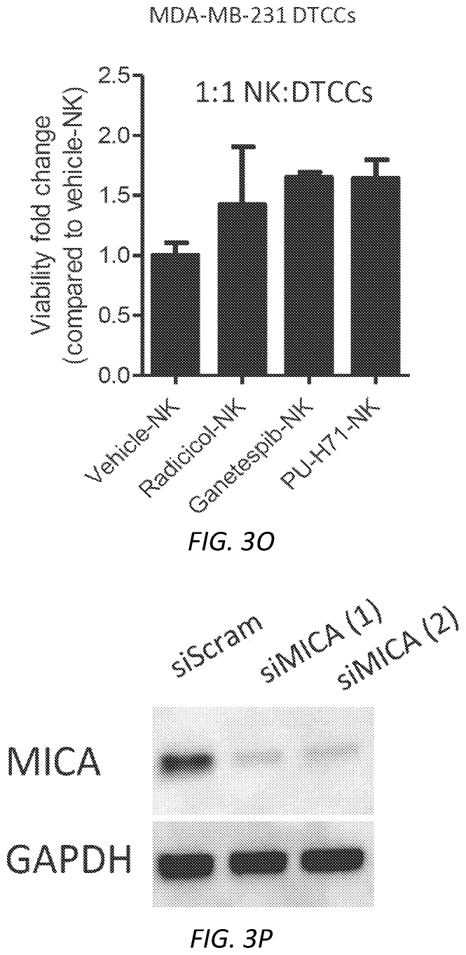

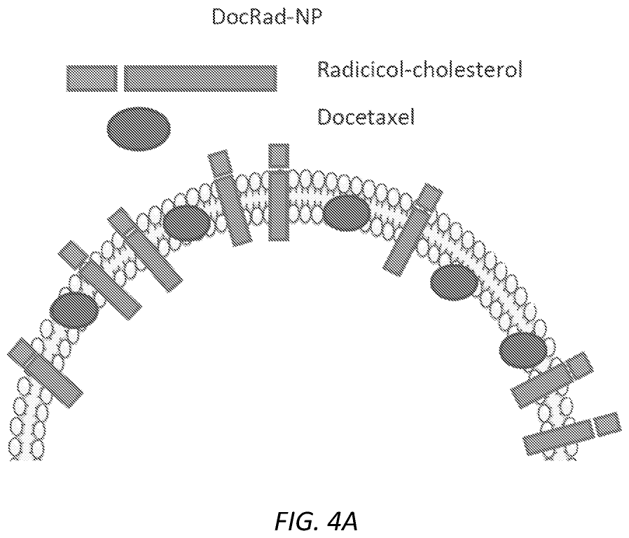

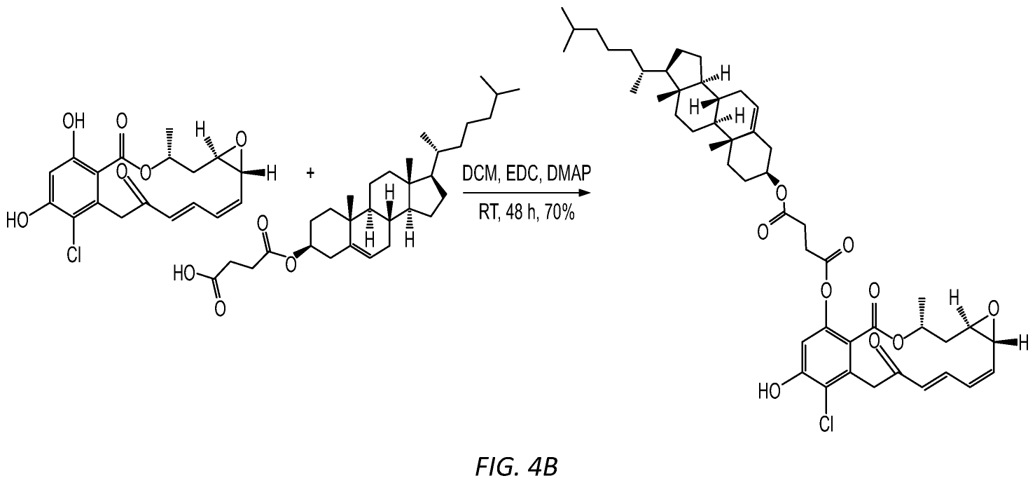

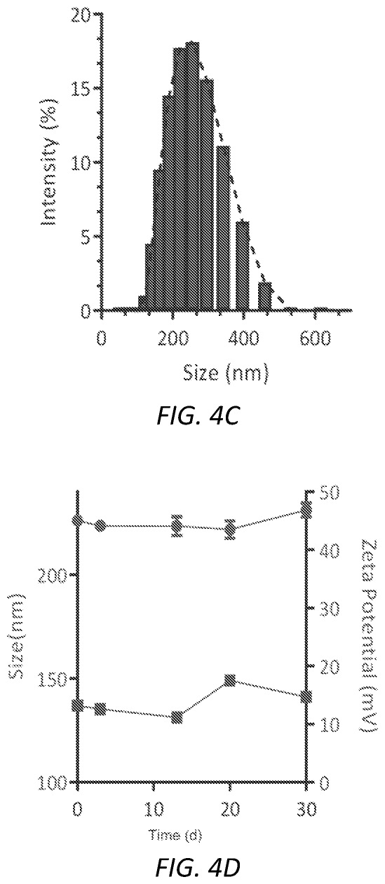

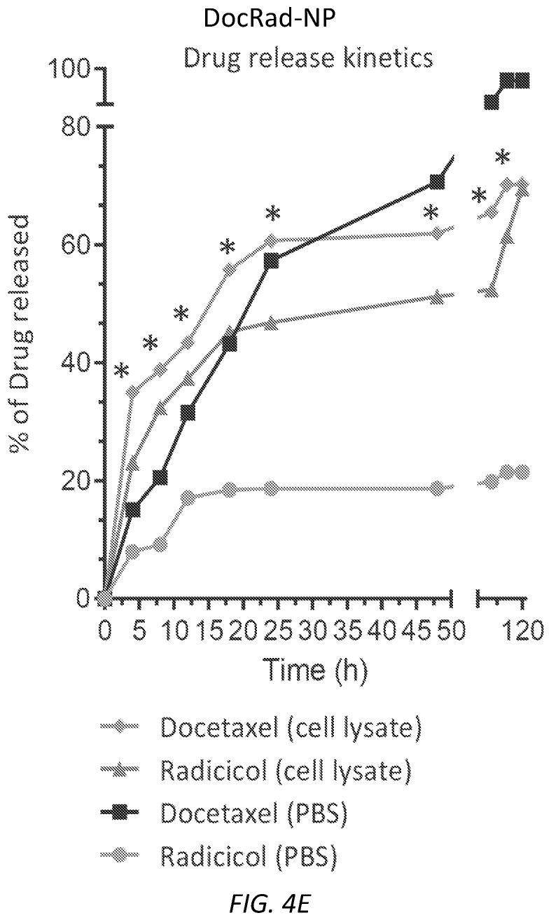

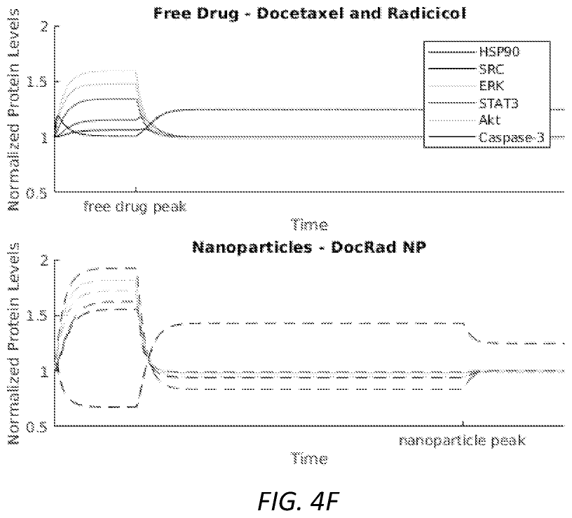

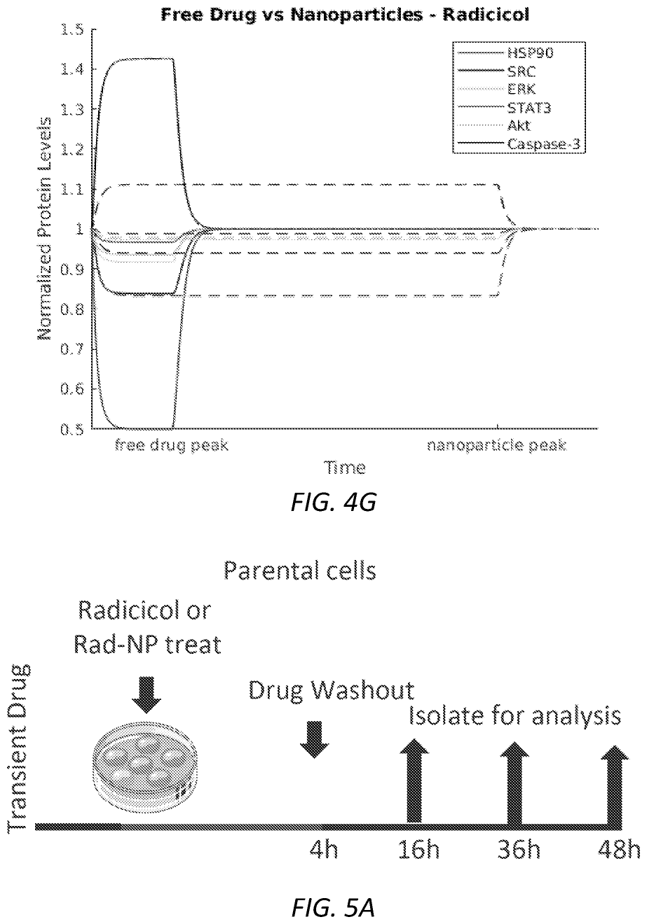

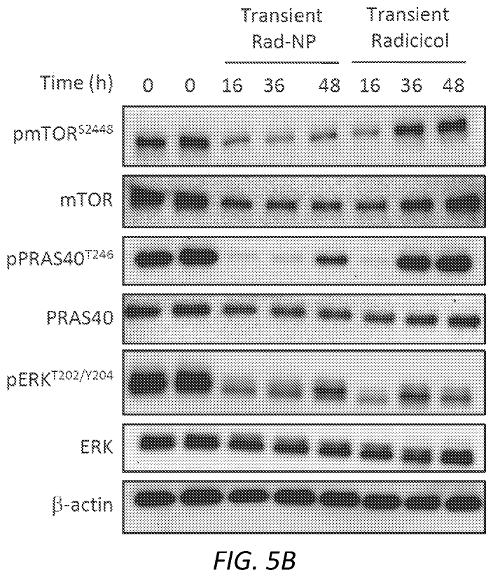

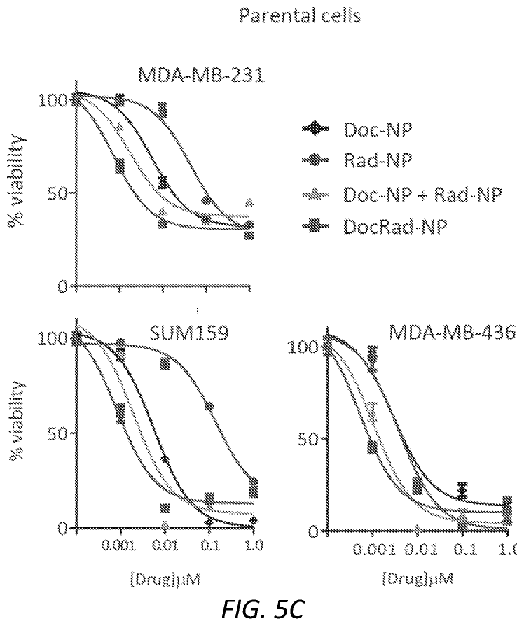

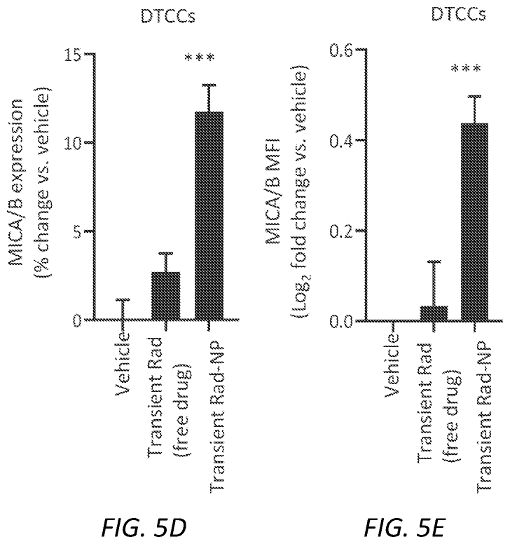

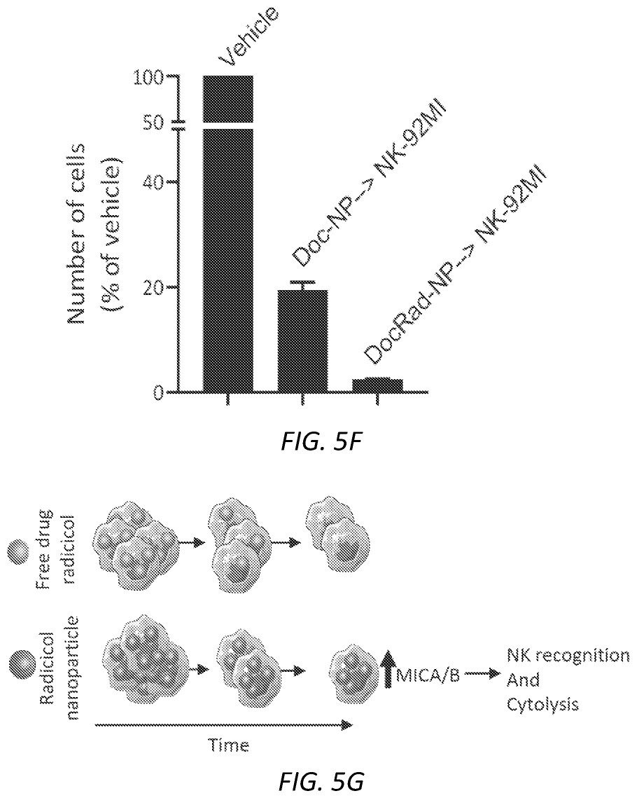

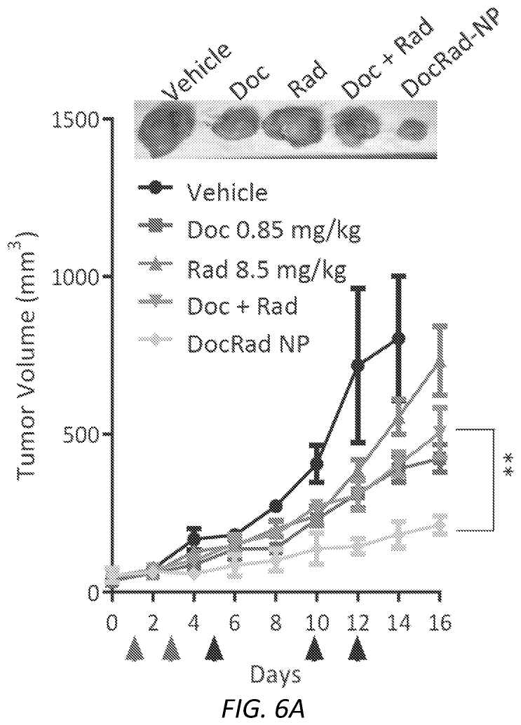

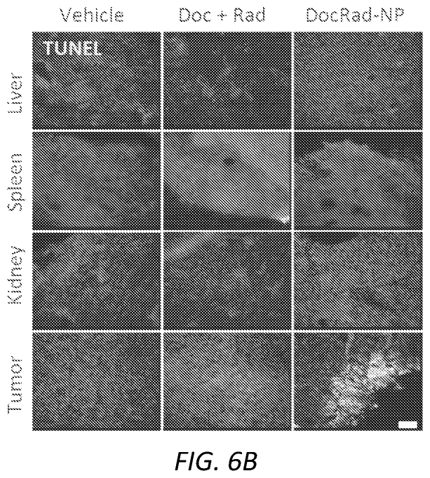

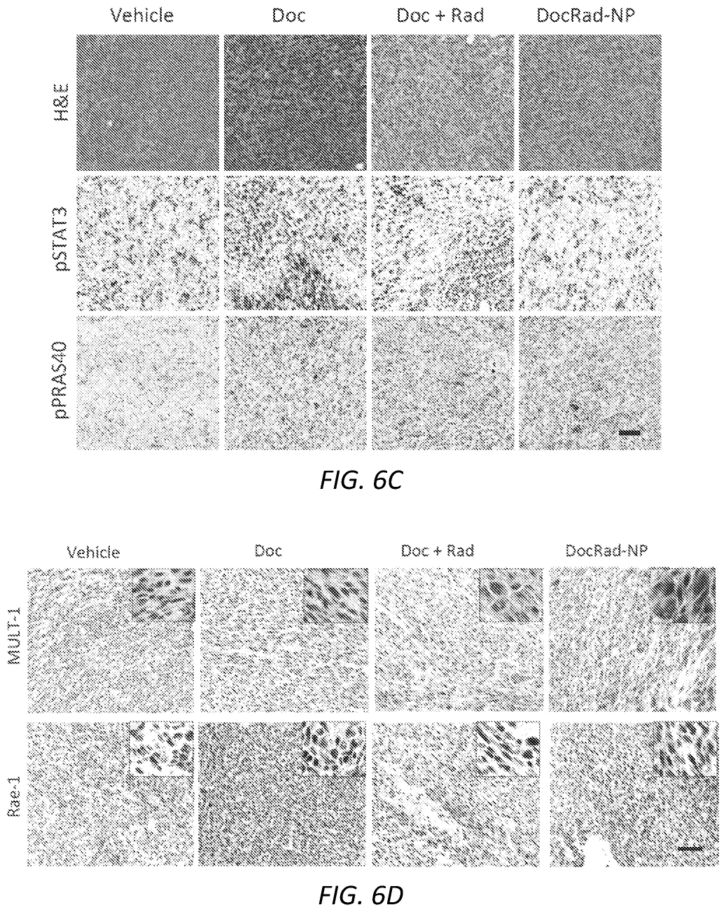

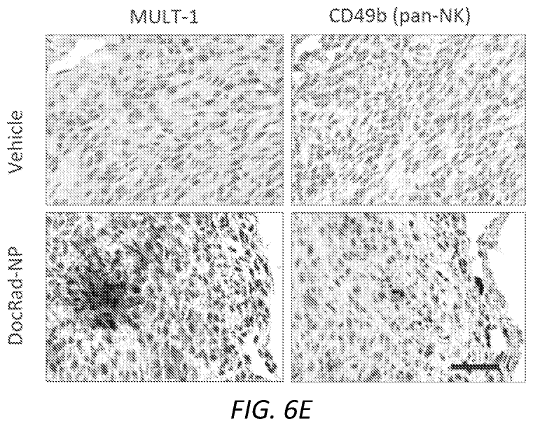

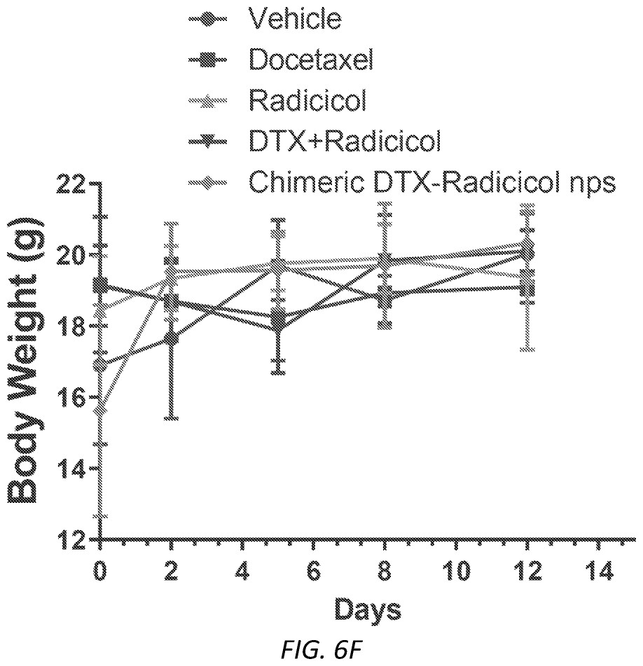

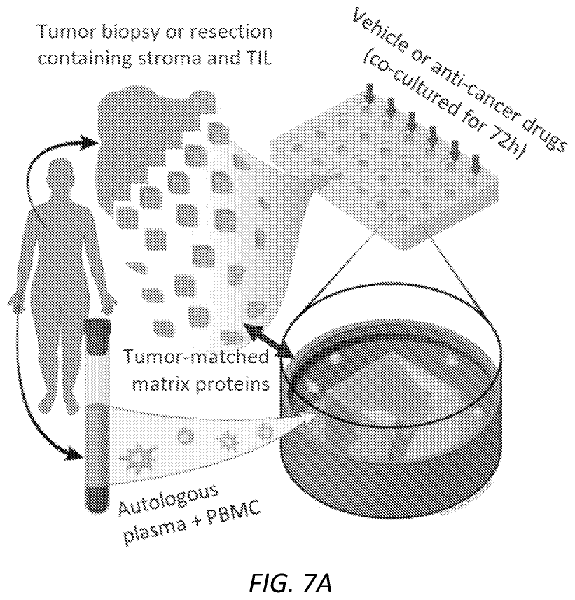

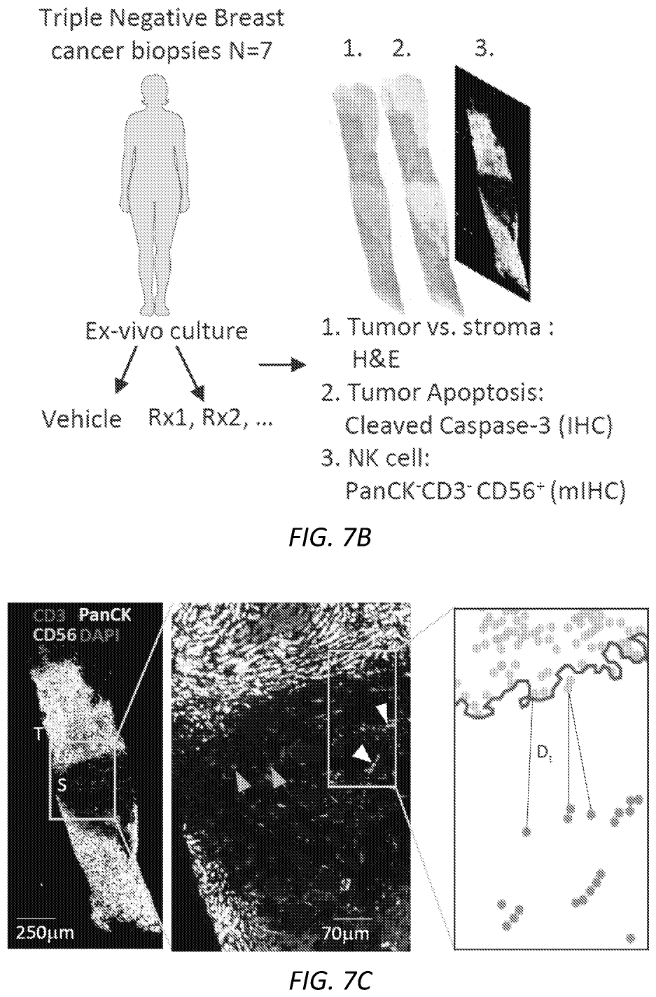

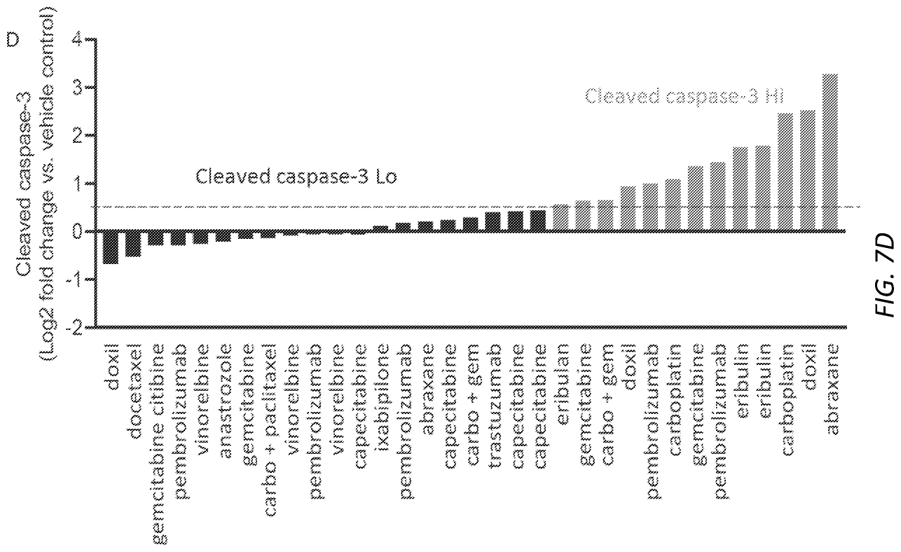

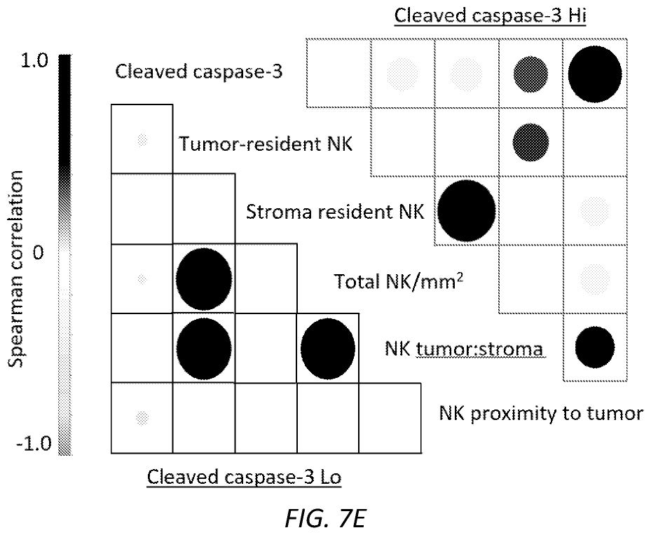

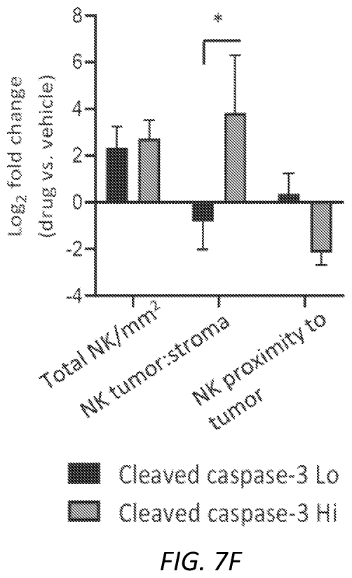

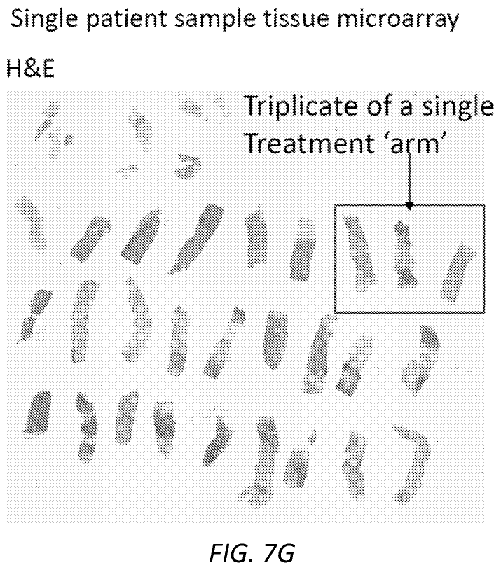

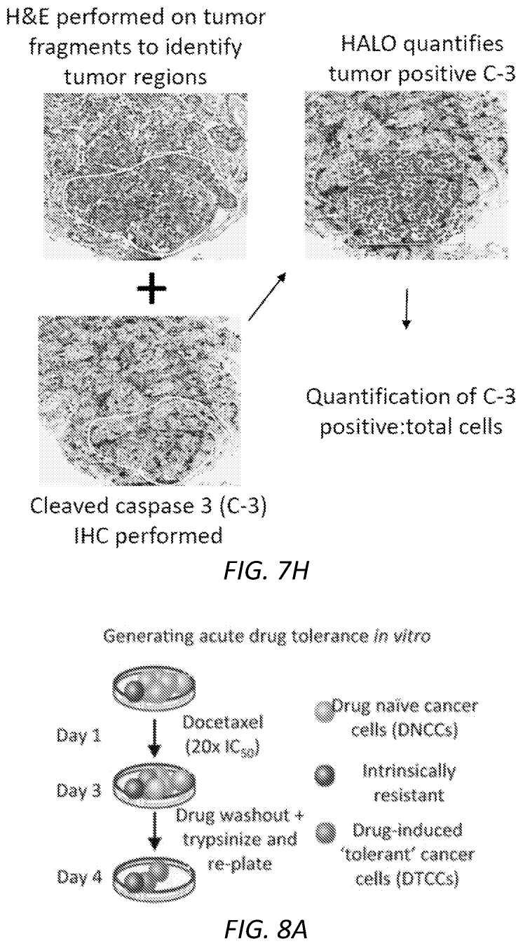

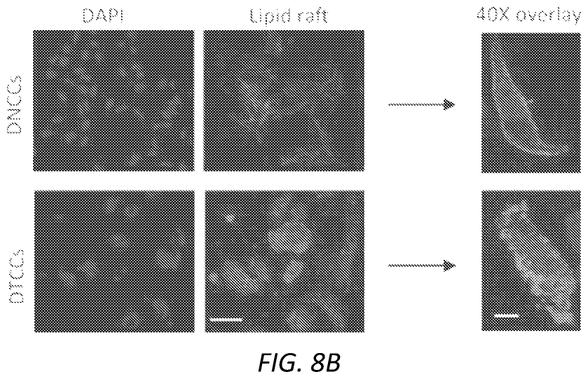

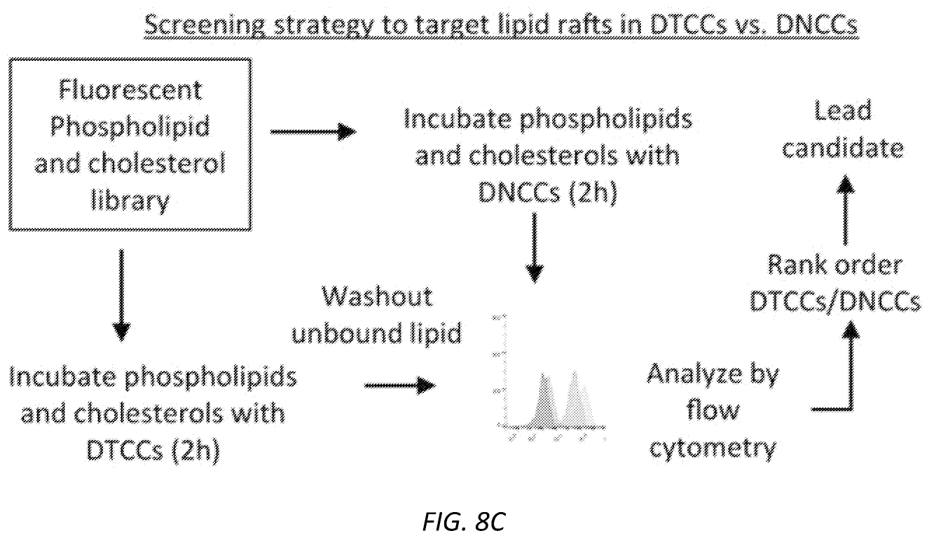

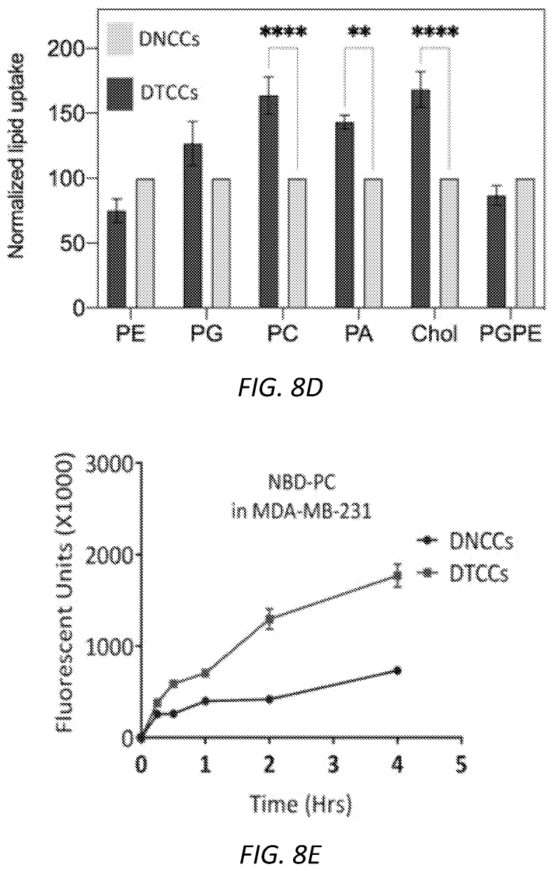

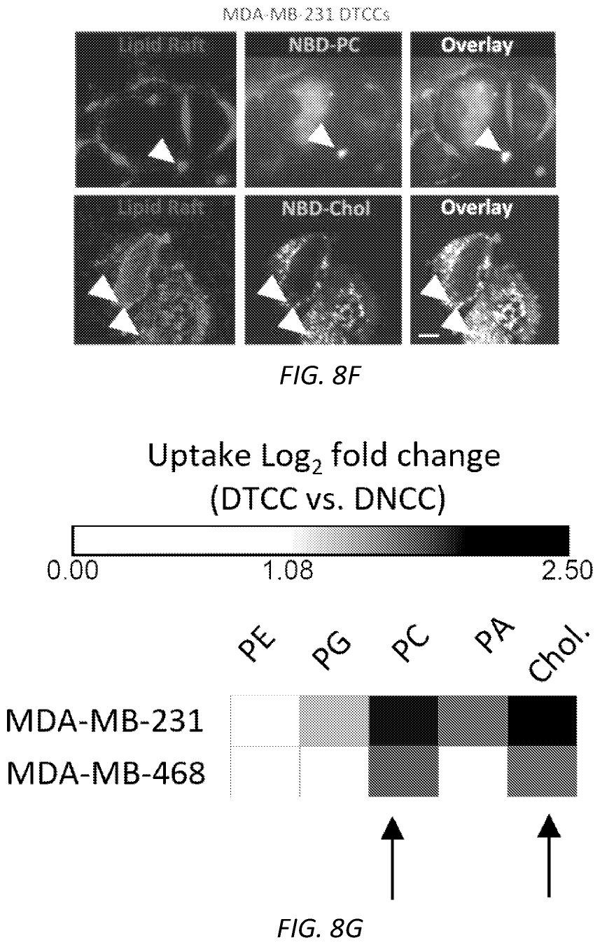

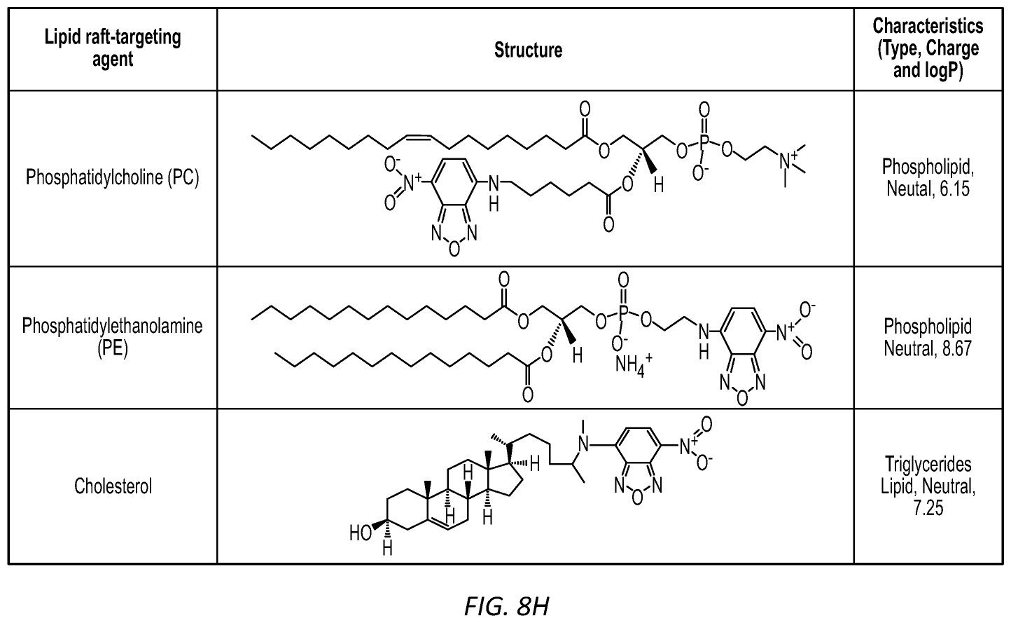

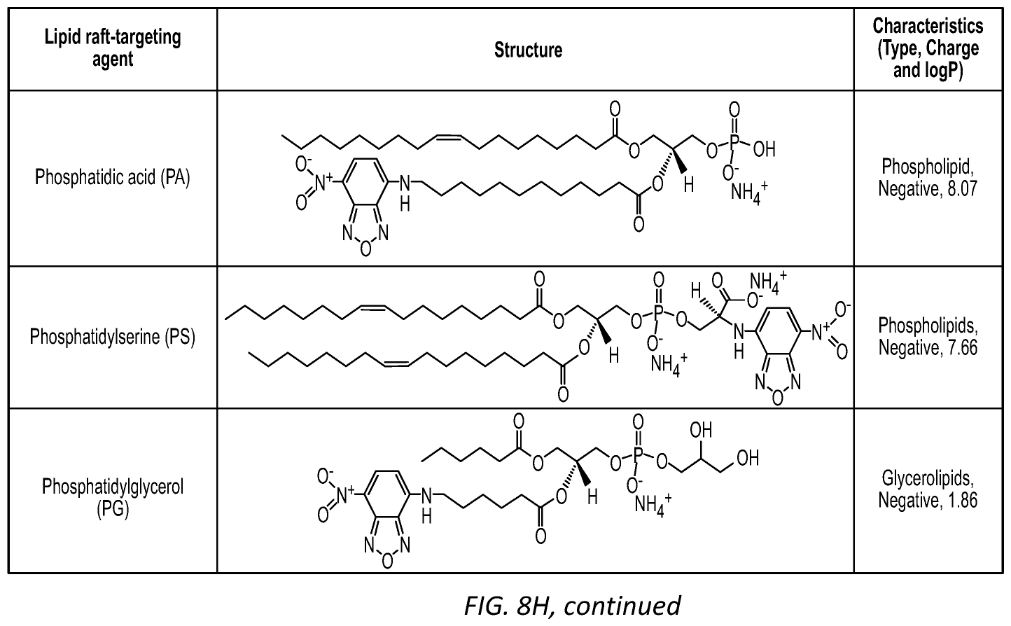

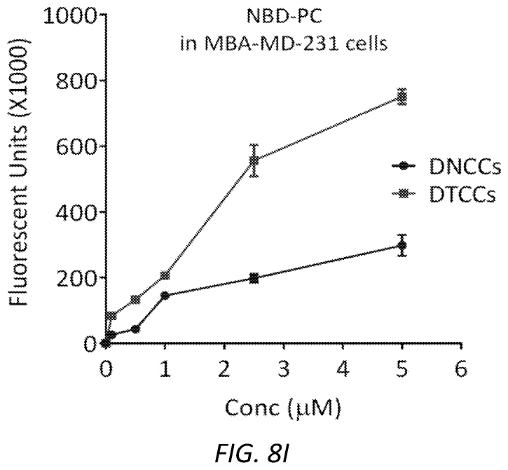

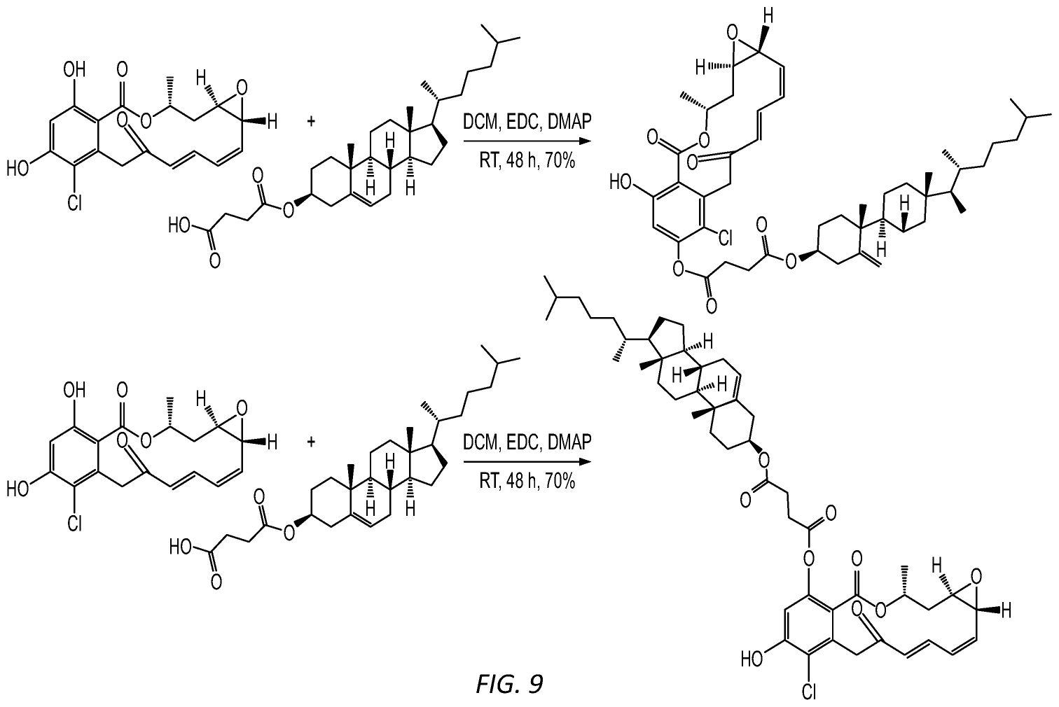

Cholesterol (500 mg, 1.29 mmol) was dissolved in 5 mL of anhydrous pyridine. Succinic anhydride (645 mg, 6.45 mmol) and catalytic amount of DMAP was added to the reaction mixture to form a clear solution. The reaction mixture was stirred for 12 hours under argon atmosphere. Removal of pyridine was carried out under vacuum and the crude residue was diluted in 30 mL DCM, washed with 1N HCl (30 mL) and water (30 mL) and the organic layer was separated and dried over anhydrous sodium sulfate, filtered and concentrated in vacuo. Completion of the reaction was confirmed by TLC in 1:99 Methanol: DCM solvent mixture. Radicicol (25 mg) was dissolved in 2 mL anhydrous DCM followed by addition of 1.2 M equivalent of cholesterol hemisuccinate, EDC and DMAP. The reaction mixture was stirred at room temperature for 48 hours under argon. Upon completion of reaction as monitored by TLC, the solvent was evaporated under vacuum and the crude product was purified by column chromatography, eluting with DCM: methanol gradient, to give radicicol-cholesterol conjugate as a yellow solid. The obtained conjugate was analyzed by ¹H NMR and Mass spectrometry. See FIG. 9 for reaction schemas).

### Synthesis and Characterization of Supramolecular Nanoparticles (SNPs)

Docetaxel, Radicicol-cholesterol conjugate, L-α-phosphatidylcholine, and DSPE-PEG2000 at 0.01:0.09:0.6:0.3 molar ratios were dissolved in 1.0 mL DCM. Resulting clear solution was evaporated and thoroughly dried. The resulting thin film was hydrated with PBS with constant rotation at 60° C. for 1 hours to get white turbid solution containing supramolecular nanoparticles (SNPs). SNPs were eluted through a Sephadex column and extruded through 0.4 μm polycarbonate membrane using mini-extruder. 10 μL of nanoparticles solution was diluted to 1 mL using DI water and 3 sets of 10 measurements each were performed at 90 degree scattering angle to get the average particle size by Dynamic Light Scattering method using Zetasizer Nano ZS90 (Malvern, UK). The zeta potential was measured using a Zetasizer ZS90 with the nanoparticles diluted in water for measurement according to the manufacturer's manual.

Release Kinetics Studies

Drug loaded supramolecular nanoparticles (1 mg drug/mL, 5 mL) were suspended in PBS buffer (pH 7.4), 4T1 cell lysate and sealed in a dialysis tube (MWCO=3500 Dalton, Spectrum Lab). The dialysis tube was suspended in 1 L PBS (pH 7.4) with gentle stirring to simulate the infinite sink tank condition. A 100 μL portion of the aliquot was collected from the incubation medium at predetermined time intervals and replaced by equal volume of PBS buffer, and the released drug was quantified by HPLC and plotted as cumulative drug release.

Materials

All reactions were performed under inert conditions unless otherwise indicated. Dichloromethane (DCM), anhydrous DCM, Methanol, Cholesterol, Dimethylamino Pyridine (DMAP), Succinic Anhydride, Sodium Sulfate, Pyridine, 1-Ethyl-3-(3-dimethylaminopropyl)carbodiimide (EDC), L-α-phosphatidylcholine and Sephadex G-25 were purchased from Sigma-Aldrich. Docetaxel (DTX) was purchased from LC laboratories. 1,2-Distearoyl-sn-Glycero-3-Phosphoethanolamine-N-[Amino(Polythylene Glycol)2000] and the mini handheld Extruder kit (including 0.2 μm Whatman Nucleopore Track-Etch Membrane, Whatman filter supports and 1.0 mL Hamiltonian syringes) were bought from Avanti Polar Lipids Inc. Analytical thin-layer chromatography (TLC) was performed using precoated silica gel Aluminum sheets 60 F₂₅₄ bought from EMD Laboratories. Column chromatography was conducted using silica gel (230-400 mesh) from Qualigens. ¹H NMR spectra were recorded on Bruker DPX 400 MHz spectrometer. Spectra were analyzed with Mest-Re—C Lite (Mestrelab Research) and/or XWinPlot (Bruker Biospin). Electrospray ionization mass spectra were recorded on a Micromass Q Tof 2 (Waters) and data were analyzed with MassLynx 4.0 (Waters) software.

Multiplex IHC (mIHC) Image Analysis

The H&E stains were annotated digitally by a clinical pathologist (David Goldman, MD, co-author) to designate tumor tissue, non-tumor tissue and stromal areas using the HALO™ digital image analysis software version 2.3.1.2089.70 (Indica Labs, Corrales, NM, USA). These annotations were then copied directly across from the H&E .tiff files using the annotations tools within HALO to the multiplex fluorescent .tiff files to establish tumor, non-tumor and stroma ROI (regions of interest). Each tumor fragment in the whole slide TMA was then segregated for individual tumor section analysis through annotation layers. Pan-CK positive staining in multiplex images was used to assist tumor designation. Using the HALO tissue classifier module version 2.0 (Indica Labs, Corrales, NM, USA) all ROI groups were input as separate classes in a random forest algorithm and ground truths (definite positive stain examples) were designated as training regions for quantitative assessment of tissue areas. Adjustments were made by training border regions to increase overall algorithm accuracy. The High Plex FL version 3.0 (Indica Labs, Corrales, NM, USA) module was used to segment cell populations within the previously designated ROI with thresholds based on the same ground truths established from the classifier training. Recognition and threshold scoring was optimized using the DAPI stain through adjustment of nuclear contrast, segmentation aggressiveness and nuclear intensity parameters. CD56⁺ and CD3⁺FL signals were then interpreted by matching to DAPI phenotyped cells due to the unsuitability of CD marker segmentation. After optimization of all fluorescent signals and phenotyping of cells the the High Plex FL algorithm was tested against a sample set of 20% of the total section library and subsequently adjusted for optimum segmentation and phenotyping. Due to the heterogeneity and differing cancer type of the patient samples alternative algorithm versions were developed for each patient as to ensure accuracy. Once all algorithms had been fully developed, there were bulk applied to the appropriate patient, establishing a data set identifying the absolute count and spatial distribution of DAPIPanCK⁻ CD56⁺CD3⁻ cells in tumor, non-tumor and stromal ROI.

Following segmentation and phenotyping the HALO Spatial analysis (Indica Labs, Corrales, NM, USA) module was used for plotting the NK data set containing the requisite X and Y coordinate map. Nearest neighbor analysis was performed in all ROIs between NK cells, while proximity analysis of NK to tumor area was calculated within stromal ROIs. Density analysis was also performed by dividing the total counts of the NK populations in each ROI by the respective ROI area (mm²) in the corresponding tumor fragment. In order to compare between fragments and patient samples, analysis of NK cell distance (i.e. proximity) to tumor interface was performed as a ratio to the stromal area per tissue fragment analyzed. Similarly, the analysis of NK in the tumor vs. stroma (i.e. tumor:stroma) was performed as a determination of the total number of NK cells in the tumor or stroma, which is normalized to the tumor or stroma area, respectively, and a ratio is obtained from that normalized value. For spearman correlation analysis, data were normalized and read into the R statistical computing package “car”. Data tables were created caspase 3 high and caspase 3 low samples (respectively). These data were fed into a variety of visualization packages (GGPlot, GGPairs, scatterplotMatrix, and corr). For Correlation analysis—The R corrplot package was utilized to visually interpret the output from the above analysis. Spearman output was visualized by creating a heatmap to express the individual correlation values that were observed.

### In Silico Model Construction

a) Key interactions

Based on a screen of the literature, Hsp90 plays a key role in promoting Src, ERK, and Akt activity. Hsp90 modulates Src activity, specifically the transportation of Src into the plasma membrane where Src is activated[1]. Hsp90 indirectly regulates ERK activity, and Hsp90 inhibition results in a decrease of activated ERK through the Raf-MEK-ERK pathway[2]. Finally, Hsp90 supports and regulates Akt activation as part of its function of apoptosis regulation[3][4]. In our model, we consider these reactions by having Hsp90 activate Src, ERK, and Akt. It should be noted that the formation of the protein complex between Hsp90 and Akt has been shown to extend the half-life of Akt[5]. This is not explicitly included in our model since the minimal model only considers proteins in the activated state, as discussed below in the Mathematical model section. Incorporating this information is one of the ways in which the presented model could be extended.

We also consider the interactions between these other proteins. Src plays a key role in activating ERK, STAT3, and Akt activity. Src kinase is an activator of ERK since it modulates growth factor-induced activation of the MAPK cascade[6]. Src directly binds with STAT3, leading to phosphorylation and activation of STAT3[7], in the cellular processes of cell growth and transformation[8]. It has also been shown that STAT3 inhibition is associated with decreased Src activation[9, 10]. Src is an activating kinase upstream of Akt[11], and inhibition of Src signaling results in decreased Akt[10]. In our model, we consider these reactions by having Src activate ERK, STAT3, and Akt. ERK activates STAT3, and STAT3 may mediate ERK activation through cytokines[12]. In our model, we include this by having ERK and STAT3 activate each other. Also, STAT3 activation contributes to Akt phosphorylation, and STAT3 inhibition results in less Akt activity[13]. In our model, we include this by having STAT3 activate Akt. Since Hsp90, ERK, STAT3, and Akt are prosurvival proteins, we have each of these proteins inhibiting caspase-3 in our model. In addition, an increase in caspase-3 has been observed with a decrease in Akt levels[14], so our model includes caspase-3 inhibiting Akt.

b) Drug effects

At the beginning of the in silico experiments, the initial cell population has not been treated with any drugs, i.e. the cells are in a drug-naïve state. However, we are considering a population of drug-tolerant cells (DTCC), which include cells that may acquire drug resistance and cells that may have intrinsic drug resistance. The drugs under consideration are a cytotoxic drug, docetaxel, and an Hsp90-inhibitor, radicicol. Docetaxel administration, results in an activation of ERK and Akt[15]. Since resistance to docetaxel has been seen to be associated with increased expression of Hsp90[16, 17], and the data obtained in this study indicates Hsp90 is increased in DTCC compared to parental cells, we assume that DTCC have increased Hsp90 levels. We also assume that docetaxel causes a cell to become more dependent on the Hsp90 survival pathway and less dependent on other survival and anti-apoptotic pathways.

Radicicol works through Hsp90-inhibition, which has anti-proliferative effects and results in cell death in cancer cells but not normal cells[3]. Since Hsp90 regulates many critical proteins, Hsp90 inhibition has the potential to inhibit a range of critical cancer pathways, leading to the degradation of pro-survival proteins[2]. Specifically, Hsp90 inhibition results in an increase in caspase-3 and caspase-7 levels and a decrease in Akt levels, all of which stop the growth of cells and lead to increased apoptosis[14]. Hsp90 inhibition also results in decreased levels of activated ERK through the Raf-MEK-ERK pathway[2]. These results solidify that Hsp90 activates ERK and Akt.

The aforementioned Hsp90-related pro-survival proteins are only a subset of the survival pathways within a cell, i.e. drug-naïve cells depend on other survival and anti-apoptotic pathways than the Hsp90-related pathway. Here, we assume that docetaxel sensitizes cells so that they are dependent solely on the Hsp90 pro-survival pathway. A DTCC that has been exposed to docetaxel will then have no dependence on other survival or anti-apoptotic pathways.

c) Mathematical Model

Using a systems biology approach, we constructed a minimal chemical reaction network based on the aforementioned proteins, interactions, and drug effects. Each protein can exist in an active or inactive state, usually corresponding to phosphorylated and dephosphorylated states. However, to reduce the complexity of the model, each protein is normalized and modelled only in its activated state. It is assumed that each protein has constant production and exponential decay, in addition to its specific interactions with other proteins and drugs. Activation of proteins is assumed to be dependent on the amount of activated protein available as well as the amount of activator protein available. Inhibition by proteins is dependent only on the amount of (inhibiting) protein available. Finally, it is assumed that protein levels are at equilibrium in untreated cells.

To model the drug dynamics, we assumed that the uptake of drug into a cell is constant which is then metabolized, leading to exponential decay. This convention neglects depletion of the drug in the microenvironment, thus assuming that the external drug concentration is constant. It is assumed that the drug is only in the cell's microenvironment for a finite amount of time, beginning with tₛₜₐᵣₜ and ending with t_{end}. This correspond to the in vitro experiments where drug is introduced into the cell microenvironment and then washed out after a specified amount of time (e.g. two days). Once the drug was washed out, it was no longer in the cell microenvironment and ceased to enter the cell. As illustrated in the chemical reaction network we assume that docetaxel activates Hsp90, ERK, and Akt and that radicicol inhibits Hsp90. We assume that the activation/inhibition rate is dependent on the amount of drug available in the cell. The equations describing the drug dynamics are given by:

$$\begin{matrix}\frac{d [ Doc ]}{dt} = {b}_{D} ( t ) - {d}_{D} [ Doc ] \\ {b}_{D} ( t ) = \{ \begin{matrix}{b}_{D} & {t}_{start} \leq t \leq {t}_{end} \\ 0 & otherwise\end{matrix}\end{matrix}$$

$$\begin{matrix}\frac{d [ Rad ]}{dt} = {b}_{R} ( t ) - {d}_{R} [ Rad ] \\ {b}_{R} ( t ) = \{ \begin{matrix}{b}_{R} & {t}_{start} \leq t \leq {t}_{end} \\ 0 & otherwise\end{matrix}\end{matrix}$$

As stated earlier (see above (b) Drug effects), we assumed docetaxel causes cells to become solely dependent on the Hsp90-dependent chemical network for survival. To model this, an additional species, X, was included in the model to represent the other survival and anti-apoptotic pathways that are independent of the Hsp90-dependent network. This species, X, is assumed to be constant until docetaxel treatment. As docetaxel sensitizes the cell, X is removed from the network since it is no longer relevant or necessary for the cell's survival. Since we use an inhibitor of Hsp90, it is clear that the effects are directly related to the inhibition of this protein itself. Moreover, this protein directly interacts with the survival signaling molecules in the model, which further supports this conclusion.

These reaction rates were used to construct a system of ordinary differential equations representing the protein and drug dynamics. The naming conventions for the model parameters are as follows: bₚᵣₒₜₑᵢₙ for production constants, bₚᵣₒₜₑᵢₙ₂ for inhibition constants, dₚᵣₒₜₑᵢₙ for decay constants, k_{reacting protein-activated protein} for reaction constants, α_{reacting protein-inhibited protein} for inhibition constants, xₚᵣₒₜₑᵢₙ for activation by docetaxel, and β_{reacting protein-inhibited protein} for removal of protein from the model. The time evolution of the proteins in the network are described by the following:

$$\frac{d [ Hsp 90 ]}{dt} = \frac{{b}_{H 1} + {x}_{H} [ Doc ]}{{b}_{H 2} + {α}_{RH} [ Rad ]} - {d}_{H} [ Hsp 90 ]$$

$$\frac{d [ Src ]}{dt} = {b}_{Sr} + {k}_{HSr} [ Hsp 90 ] [ Src ] - {d}_{Sr} [ Src ]$$

$$\frac{d [ ERK ]}{dt} = {b}_{E} + {x}_{E} [ Doc ] + {k}_{HE} [ Hsp 90 ] [ ERK ] + {k}_{SrE} [ Src ] [ ERK ] + {k}_{StE} [ STAT 3 ] [ ERK ] - {d}_{E} [ ERK ]$$

$$\frac{d [ STAT 3 ]}{dt} = {b}_{St} + {k}_{SrSt} [ Src ] [ STAT 3 ] + {k}_{ESt} [ ERK ] [ STAT 3 ] - {d}_{St} [ STAT 3 ]$$

$$\frac{d [ Akt ]}{dt} = \frac{{b}_{A 1} + {x}_{A} [ Doc ]}{{b}_{A 2} + {α}_{CA} [ Casp 3 ]} + \text{} {k}_{HA} [ Hsp 90 ] [ Akt ] + \text{} {k}_{SrA} [ Src ] [ Akt ] + {k}_{StA} [ STAT 3 ] [ Akt ] - {d}_{A} [ Akt ]$$

$$\frac{d [ Casp 3 ]}{dt} = \frac{{b}_{C 1}}{\begin{matrix}{b}_{C 1} + {α}_{HC} [ Hsp 90 ] + {α}_{EC} [ ERK ] + \\ {α}_{StC} [ STAT 3 ] + {α}_{AC} [ Akt ] + {α}_{XC} [ X ] - {d}_{C} [ Casp 3 ]\end{matrix}}$$

$$\frac{d [ X ]}{dt} = {β}_{DX} [ X ] [ Doc ]$$

With regards to the effect of the drug on the chemical reaction network, we made the following assumptions: a) the initial population of cells are in a drug-naïve state; b) as the cells move through the treatment, DTCC are present which are a mixture of intrinsically resistant cells and acquired resistant cells; c) cells in the presence of drug are not proliferating due to stress; d) docetaxel activates Hsp90, ERK and Akt; e) docetaxel-treated cells become more dependent on the Hsp90 pro-survival pathway; and f) radicicol inhibits Hsp90.
d) Parameter Estimation

| # | Name | Parameter Description | Units | Value |
|---|---|---|---|---|
| 1 | b_{H2} | Inhibition scaling constant for Hsp90 | — | 8.54 |
| 2 | b_{A2} | Inhibition scaling constant for Akt | — | 15.93 |
| 3 | b_{C2} | Inhibition scaling constant for | — | 0.19 |
| | | Caspase-3 | | |
| 4 | b_{D} | Intake rate of docetaxel | mol/hr | 2.06 |
| 5 | b_{R} | Intake rate of radicicol | mol/hr | 1 |
| 6 | d_{H} | Decay rate of Hsp90 | hr⁻¹ | 1.28 |
| 7 | d_{Sr} | Decay rate of Src | hr⁻¹ | 1.26 |
| 8 | d_{E} | Decay rate of ERK | hr⁻¹ | 16.69 |
| 9 | d_{St} | Decay rate of STAT3 | hr⁻¹ | 2.87 |
| 10 | d_{A} | Decay rate of Akt | hr⁻¹ | 17.61 |
| 11 | d_{C} | Decay rate of Caspase-3 | hr⁻¹ | 7.7 |
| 12 | d_{D} | Decay rate of docetaxel | hr⁻¹ | 0.20 |
| 13 | d_{R} | Decay rate of radicicol | hr⁻¹ | 0.26 |
| 14 | k_{HE} | Activation rate of ERK by Hsp90 | hr⁻¹ | 1.6 |
| 15 | k_{HSr} | Activation rate of Src by Hsp90 | hr⁻¹ | 0.35 |
| 16 | k_{HA} | Activation rate of Akt by Hsp90 | hr⁻¹ | 1.36 |
| 17 | k_{SrE} | Activation rate of ERK by Src | hr⁻¹ | 0.53 |
| 18 | k_{SrSt} | Activation rate of STAT3 by Src | hr⁻¹ | 1.00e⁻⁶ |
| 19 | k_{SrA} | Activation rate of Akt by Src | hr⁻¹ | 0.85 |
| 20 | k_{ESt} | Activation rate of STAT3 by BERK | hr⁻¹ | 1 |
| 21 | k_{StE} | Activation rate of ERK by STAT3 | hr⁻¹ | 1.08 |
| 22 | k_{StA} | Activation rate of Akt by STATS | hr⁻¹ | 0.07 |
| 23 | α_{HC} | Inhibition strength of Hsp90 on | — | 6.43 |
| | | Caspase-3 | | |
| 24 | α_{EC} | Inhibition strength of ERK on | — | 0.38 |
| | | Caspase 3 | | |
| 25 | α_{StC} | Inhibition strength of STAT3 on | — | 1 |
| | | Caspase | | |
| 26 | α_{AC} | Inhibition strength of Akt on | — | 1.06 |
| | | Caspase-3 | | |
| 27 | α_{CA} | Inhibition strength of Caspase-3 on | — | 1.42 |
| | | Akt | | |
| 28 | α_{RH} | Inhibition strength of radicicol on | mol⁻¹ | 2.25 |
| | | Hsp90 | | |
| 29 | α_{XC} | Inhibition strength of X on | — | 2.22 |
| | | Caspase-3 | | |
| 30 | x_{H} | Activation constant of docetaxel on | mol⁻¹ · hr⁻¹ | 1.4 |
| | | Hsp90 | | |
| 31 | x_{E} | Activation constant of docetaxel on | mol⁻¹ · hr⁻¹ | 0.54 |
| | | ERK | | |
| 32 | x_{A} | Activation constant of docetaxel on | mol⁻¹ · hr⁻¹ | 14.63 |
| | | Akt | | |
| | b_{H1} | (Uninhibited) “production” rate of | hr⁻¹ | 10.91 |
| | | Hsp90 | | |
| | b_{Sr} | ″Production″ rate of Src | hr⁻¹ | 0.91 |
| | b_{E} | “Production” rate of ERK | hr⁻¹ | 13.48 |
| | b_{St} | “Production” rate of STAT3 | hr⁻¹ | 1.87 |
| | b_{A1} | (Uninhibited) “production” rate of | hr⁻¹ | 265.92 |
| | | Akt | | |
| | b_{C1} | (Uninhibited) “production” rate of | hr⁻¹ | 86.83 |
| | | Caspase-3 | | |
| | β_{DX} | “Removal” rate of X | mol⁻¹ · hr⁻¹ | 1 |

Once the final model was constructed, the genetic algorithm in MATLAB was used to explore the multi-dimensional parameter space to find a local minimum for the error between the simulation results and the normalized quantification of the Western blots from the docetaxel-radicicol treatment sequence. Given the large parameter space, the following constraints were used to limit the algorithm to relevant parameter possibilities: a) parameters must be strictly positive so that every reaction is accounted for in the model; b) the network is at equilibrium in the drug-naïve cell; c) drug decay rates must be sufficiently slow to ensure that there is a lingering effect from the drug once the drug is no longer being taken in by the cell; d) drug decay rates must be sufficiently fast to ensure that drug levels decrease after the drug is no longer being taken in by the cell; and e) drug intake rates must be sufficiently slow to ensure that the protein levels do not saturate too early, i.e. the protein dynamics are changing/saturating in a relevant timescale. Although the parameter fit was not unique, it was sufficiently close to model the protein dynamics and within the range of biologically relevant parameters as compared to similar models in the literature. Additionally, the removal rate of X was set arbitrarily to ensure a smooth and complete transition to a docetaxel-treated cell that is dependent only on the Hsp90-dependent survival pathways. It should be noted that these parameter values may not be within the biological range since the mathematical model is mostly phenomenological and simplifies the interactions between the prosurvival proteins of interest. In other words, there may be multiple reactions and interactions captured in a single parameter.

e) Sensitivity Analysis

Local sensitivity analysis of the caspase-3 extremum during the docetaxel-radicicol treatment schedule was completed to identify the key parameters in bringing about cell death as well as to confirm that our parameters resulted in a realistic representation. Each parameter was positively perturbed individually by 1% of its nominal value, and the resulting parameter sets were simulated. In each simulation, the extremum of caspase-3 (from steady state) was calculated and then used to calculate the change in caspase-3 extremum (from its nominal value, i.e. using the original parameter fit). The change in caspase-3 extremum was divided by the change in the parameter to give us the absolute sensitivity of each parameter. This value was then divided by the nominal caspase-3 extremum to calculate the relative sensitivities. The absolute value of each of these relative sensitivities was then calculated to identify the key parameters. Local sensitivity analysis was repeated for different perturbation values ranging from 10⁻⁵ to 10⁻² with qualitatively similar results.

The analysis identified the four most influential parameters (ordered from most to least) as #5 intake rate of radicicol, #28 inhibition strength of radicicol on Hsp90, #13 decay rate of radicicol, and #1 inhibition scaling constant for Hsp90. Note that the parameter numbers correspond to the table of fit parameters. The inhibition scaling constant for Hsp90 is inherent to the nature of Hsp90, which may be outside of our control. However, the intake rate, decay rate and inhibition strength of radicicol indicate the importance of radicicol as a follow-up drug to docetaxel in this new treatment sequence. Improving the efficacy of radicicol on Hsp90 in the docetaxel-radicicol treatment sequence may improve this treatment.

f) Drug Delivery Comparison

Nanoparticle (NP) drug delivery and free drug delivery for docetaxel and radicicol and radicicol alone were simulated for comparison. For both cases, the drug was administered at the beginning of the experiment. The only difference between the two administrations was the slower release of radicicol into the cell microenvironment for the NP drug delivery due to the cholesterol binding to radicicol in the NP design. This was modelled by decreasing the intake rate of radicicol and increasing the time for which the drug is being taken into the cell so that the total amount of drug is the same for free drug and NP drug delivery. When we considered radicicol only, we found that the NP structure resulted in a lesser but longer lasting effect. For docetaxel and radicicol together, we found that the two-drug nanoparticles (DocRad-NP) took greater advantage of the sensitizing effect of docetaxel. In the free drug case, both drugs were in effect concurrently, allowing for radicicol to take effect before the cell was completely sensitized to Hsp90. In the case of the NP, due to the slow-release kinetics of radicicol, docetaxel is able to completely sensitize the cell to Hsp90. Thus, there was more time for radicicol to act on the sensitized cell, resulting in increased caspase-3 levels.

Statistics

Statistical analysis was performed using Prism software (GraphPad) determined by ANOVA analysis followed by a Newman-Keuls post hoc test when values were represented between multiple groups, and, unless otherwise noted, two-tailed Student's t-test used to identify statistical significance between individual groups. 2-way ANOVA was employed to track significance between groups from in vivo tumor volume experiments.

### Example 1.1. Drug-Induced Resistant Cancer Cells Diminish Immune Surveillance and Cytolytic Activity of NK Cells Following Induction of Granulocyte Stimulating Cytokines, In Vitro

To interrogate the activity of NK cells in the presence of drug naïve vs. drug-induced resistant (i.e. tolerant) TNBC cells, we deployed an in vitro co-culture model. We generated a population of drug tolerant cancer cells (hereafter referred to as DTCCs) that temporarily display a hybrid epithelial-mesenchymal cell state implicated in therapy failure in multiple humanized models (12). Briefly, the parental TNBC cell line, MDA-MB-231, was exposed to docetaxel, a routine cancer chemotherapy for TNBC (26), at >20× the published IC₅₀ (FIG. 1A). We co-cultured constitutively active NK cells (NK-92MI) or CD56+ primary human peripheral blood NK cells with either parental cells or DTCCs to assess cytolytic activity (FIG. 1B). We determined that NK cells were incapable of killing DTCCs compared to parental cells across multiple TNBC models tested with different genetic backgrounds (FIG. 1C). To study NK cell activity, we used a co-culture experimental design in which tumor spheroids, generated using a NanoCulture system (27), are separated from NK cells by a 0.2 μm porous membrane that restricts diffusion to secreted factors such as growth factors, cytokines, chemokines and lipids (FIG. 1D). Using flow cytometry, we evaluated several NK inhibitory and activating biomarkers including the well-established activation marker, natural killer group 2 member D (NKG2D), which plays a key role in NK activity (18). Notably, the expression of NKG2D and minimally-expressed NKG2C, which bind and elicit immune responses on ligand receptors such as MICA/B (28), were significantly diminished on NK cells in co-culture with DTCCs vs. the parental cells (FIG. 1E). In parallel, we noted MICA/B was diminished on cancer cells in response to taxanes and on DTCCs. To elucidate a mechanism underlying the decrease of NKG2D on NK cells, we isolated cell culture supernatant from parental cells or DTCCs and interrogated the excreted cytokine milieu using multiplex Luminex cytokine analysis over the course of 24 hours (FIG. 1F). Clusters of cytokines that affiliated with DTCCs vs. parental cells emerged (FIG. 1G), yet a smaller cohort was found to overlap among two independent TNBC cell lines tested, which included regulated on activation, normal T cell expressed and secreted (RANTES), granulocyte colony stimulating factor (G-CSF) and granulocyte macrophage colony stimulating factor (GM-CSF) (FIG. 1I). Based on these results, we attempted to phenocopy the DTCC microenvironment in parental cells by introducing the top-induced cytokines or a cocktail of those that clustered together by Euclidean distance in the Luminex array (i.e. VEGF, G-CSF, GRO, RANTES and ILlu). Cell viability analysis confirmed that G-CSF, GM-CSF alone and in combination with other cytokines were the only ones tested to recapitulate the DTCC TIME in a parental cell line (FIG. 1H). Indeed, using flow cytometry we confirmed a reduction of NKG2D expression on NK cells, which is consistent with reports in other physiologic contexts (29,30).

### Example 1.2. Hsp90 Simultaneously Suppresses NK Cell Recognition and Cancer Cell Survival Axes in Drug-Induced Resistant Cancer Cells

Next, we deployed systems biology and computational modeling to establish a chemical reaction network that integrated drug-induced protein kinetics with a systems biology approach. We used this strategy to infer drug effect on the DTCC phenotype using a framework that provided some mathematical certainty in which we could ‘toggle’, in silico, the effect of pathway perturbations. To do this, we interrogated phosphorylation status of proteins in DTCCs compared to parental cells and integrated these observations with empirical evidence from a kinetic analysis of drug-induced protein phosphorylation to establish the system of proteins and parameters that are induced by therapy, rather than ‘drug selected’. This approach implicated multiple protein families and pathways that are induced in the DTCCs and activated in discrete, time-dependent patterns following drug pressure in drug naïve parental cells (FIG. 2A). The systems biology and chemical reactions network that was built from these empirical observations, and from a review of the literature, identified Hsp90 as a potential ‘node’ with the closest determined relationship between a pro-survival phenotype in DTCCs as well as modulator of NK cell recognition of tumor cells, which functions via suppression of MICA/B through sequestration of the heat shock factor 1 (HSF-1) (31) (FIGS. 2B-C). We confirmed an increased expression of Hsp90 in the DTCCs compared to parental cells using fluorescent microscopy and western blot (FIGS. 2C-F) and found that docetaxel drug pressure induced the active form of HSF-1 (Ser326) (FIG. 2G) (32), which appeared to sequester in the perinuclear space of DTCCs vs. parental cells (FIG. 2G). Disruption of Hsp90 using the macrocyclic anti-fungal antibiotic, radicicol (33), or various other small molecule inhibitors including ganetespib (34) and PU—H71 (35), reversed cytoplasmic sequestration of HSF-1^{Ser326} and activation of pro-survival proteins in DTCCs as evidenced by confocal microscopy and western blot, respectively (FIG. 2G-H).

### Example 1.3. Ordering Taxanes Before Hsp90 Inhibitors Augments Anti-Cancer Effects and Re-Invigorates NK Cell Surveillance and Cytolysis, In Vitro

The timing and sequence of anticancer drug combinations is an important consideration, which influence responses to therapy (36). Indeed, simultaneous administration of taxanes in combination with targeted inhibitors was previously determined to be suboptimal (12). We tested the hypothesis that sequencing Hsp90 inhibitors and docetaxel can optimize anticancer effects while also improving immune detection by NK cells via NKG2D receptor ligand expression. We tested the anticancer effect of different dosing schedules on cancer cell viability, timing the separation of docetaxel and radicicol in discrete order (FIG. 3A). Values from cell viability analysis were used to plot the fraction affected (F(a)), which indicates the fraction of cells inhibited by treatment administered. A combination index (CI) was calculated at each F(a), CI below 1 indicates synergism and above 1 indicates antagonism (23). Schedule 2 (radicicol before docetaxel) resulted in antagonism. In contrast, schedule 1 (docetaxel before radicicol) resulted in synergism across all cell lines (FIGS. 3B and 3I). To validate these results, we simulated protein signaling and cell death by considering the reaction rates of the chemical reaction network, which were used to construct a system of ordinary differential equations to represent the protein and drug dynamics. The genetic algorithm in MATLAB was then used to explore our multi-dimensional parameter space and find a local minimum for the error between the simulation results and the in vitro data. With the given parameter fit, in silico experiments confirmed a direct correlation between docetaxel and radicicol sequencing and the effect on Hsp90 pathway induction and perturbation when drugs are administered in discrete sequence (FIG. 3J).

We next tested how Hsp90 disruption affects NK cell recognition and cytolysis in cells using the optimized temporal schedule (i.e., docetaxel before radicicol). Indeed, Hsp90 inhibition ‘primed’ tumor cells, significantly increasing both the intensity of expression and percent (%) positive-expressing MICA/B cells, (FIGS. 3C,D and Table 1). Moreover, co-cultures of NK cells with DTCCs that had been ‘primed’ were significantly more sensitive to NK cytolysis, as determined by cell viability analyses (FIGS. 3E-F and 3K-M). We confirmed this effect was indeed tumor cell-dependent by treating NK cells with Hsp90 inhibitors and determining there was no change in tumor cell cytolysis vs. vehicle control (FIG. 3N-0). Finally, we used siRNA gene knockdown of MICA (FIG. 3P) and determined that NK cells lost a significant proportion of their cytolytic capacity in MICA-knockdown DTCCs (MICA^{KD}), which we had ‘primed’ by overnight inhibition of Hsp90 (FIGS. 3G,H). The in silico modeling data together with in vitro evidence support a rationale for sequencing docetaxel prior to radicicol, but not inversely, as a means to improve antitumor effects while simultaneously re-priming tumor cells for NK cell cytolysis.

TABLE 1
Expression of MICA/B in multiple TNBC DTCC lines under
pressure of multiple Hsp90 inhibitors

| | Vehicle | Radicicol | |
|---|---|---|---|
| Cell line | MICA (%) ± SEM | MICA (%) ± SEM | P-value |
| MDA-MB-231 | 76.2 ± 1.1 | 93.2 ± 0.9 | 1.0 × 10⁻⁶ |
| DTCCs | | | |
| MDA-MB-468 | 54.7 ± 3.9 | 96.8 ± 1.8 | 1.7 × 10⁻⁴ |
| DTCCs | | | |
| SUM 159 | 30.6 ± 0.9 | 74.8 ± 0.5 | 4.0 × 10⁻⁶ |
| DTCCs | | | |
| | Vehicle | Ganetespib | |
| Cell line | MICA (%) ± SEM | MICA (%) ± SEM | P-value |
| MDA-MB-231 | 76.2 ± 1.1 | 82.5 ± 2.2 | 0.0031 |
| DTCCs | | | |
| MDA-MB-468 | 54.7 ± 3.9 | 70.1 ± 3.0 | 0.036 |
| DTCCs | | | |
| SUM 159 | 30.6 ± 0.9 | 35.4 ± 6.0 | 0.3 |
| DTCCs | | | |
| | Vehicle | PU-H71 | |
| Cell line | MICA (%) ± SEM | MICA (%) ± SEM | P-value |
| MDA-MB-231 | 76.2 ± 1.1 | 79.5 ± 0.4 | 0.0161 |
| DTCCs | | | |
| MDA-MB-468 | 54.7 ± 3.9 | 71.9 ± 4.2 | 0.042 |
| DTCCs | | | |
| SUM 159 | 30.6 ± 0.9 | 33.3 ± 4.1 | 0.3 |
| DTCCs | | | |

### Example 1.4. Characterizing 2-In-1 Nanomedicines with Rapid Release of Docetaxel and Sustained Release of the Hsp90 Inhibitor, Radicicol

Cancer nanomedicines are useful tools to differentially release drug payloads in distinct, controlled, temporal constraints(37) or co-delivery of two drugs to control spatial distribution of drugs (38). In this study, our evidence suggested that (1) drug order was important to improve the anticancer effect of the combination of docetaxel and radicicol and (2) DTCCs suppress NK cells via prolonged secretion of extracellular factors, which can be remedied by sustained inhibition of Hsp90. Given the physiological limitations of drugs, which have shortened half lives in vivo compared to in vitro cell cultures, we hypothesized that a 2-in-1 drug delivery strategy (docetaxel and radicicol chimera) would achieve two goals: (1) fast release of docetaxel will eliminate the drug sensitive population in the tumor; (2) sustained release of radicicol suppresses survival and optimally improves tumor immunity in the residual tumor population by boosting expression of NK activity ligand receptors. In silico drug delivery comparisons predicted improved anticancer outcomes using a time-delayed ‘chimeric’ approach (FIG. 4F-G). Next, we engineered a nanoparticle (NP) containing both docetaxel and radicicol (DocRad-NP) wherein radicicol is conjugated to cholesterol and held in the lipid bilayer, designed for slow release, while free-form docetaxel was encapsulated for rapid release (FIGS. 4A-B). The dual payload NP was constructed using a thin film hydration followed by an extrusion approach. Dynamic light scattering confirmed the formation of a supramolecular nanostructure of 225±42 diameter (FIG. 4C) where the ζ-potential and size were consistent over time at 4° C. (FIG. 4D). Release kinetics show minimal release of radicicol NPs in PBS (20%) when compared to release in cell lysate (65%) over 125 hours (FIG. 4E). Notably, differential release kinetics of docetaxel and radicicol were observed such that rapid release of docetaxel was seen within 4 hours compared to radicicol (35% vs. 23%, respectively) with a clear separation in release kinetics followed by saturation of docetaxel (70%) observed at 96 hours compared to equivalent level of release of radicicol (p<0.05), which was not achieved until 120 hours (FIG. 4E). We reasoned that DocRad-NP may serve as a suitable tool to selectively toggle the release of the chemotherapy agent (docetaxel) and the Hsp90 inhibitor (radicicol) in a time-dependent fashion to achieve optimal cell kill and sustained ‘priming’ of any residual cancer mass.

### Example 1.5. Sustained Release of Radicicol Primes Drug-Induced Resistance Via NKG2D Ligand Receptors in Nanoparticle Formulation Vs. Free Drug, In Vitro

Next, we characterized the pharmacodynamics and in vitro efficacy of DocRad-NP. DTCCs were treated with the radicicol-NP or free-form (free drug) followed by an immediate wash-out after 4 hours and analyzed at 16, 36 and 48 hours later by western blotting (FIG. 5A). The radicicol-NP-treated cells showed sustained inhibition of phosphorylated proteins up to and beyond 48 hours compared to free drug, which showed rescue of signaling disruption by 36 hours in most cases (FIG. 5B). Furthermore, cell viability analysis in drug naïve parental cells indicated that DocRad-NP achieved 50% cell killing at concentrations below those of the single drug-loaded NP individually or together (FIG. 5C). Next, we interrogated MICA/B expression in the context of free drug radicicol or radicicol-NP. Transient (4 h) treatment of the radicicol-NP resulted in a significant increase in the % positive expression of MICA/B on DTCCs compared to the free drug (p<0.001) and improved cytolytic capacity of NK cells in the residual tumor cell fraction (FIGS. 5D-F). These data suggested an improvement of antitumor effects while simultaneously priming of the residual tumor mass for NK surveillance (FIG. 5G).

### Example 1.6. DocRad-NPs Reduce Tumor Burden, In Vivo, and Prime Residual Tumor Cells for NK Cell Surveillance Via NKG2D Ligand Receptor Expression

Next, we used an in vivo immune-competent orthotopic syngeneic mammary carcinoma model (4T-1 in Balb/C), and treated mice with either docetaxel, radicicol, a combination of individual compounds or DocRad-NP at equivalent drug concentrations. In a previous report we showed that maximum tolerated dose of docetaxel chemotherapy in this syngeneic model will induce the DTCC phenotypic transition, in vivo (12). First, we determined that DocRad-NP displayed superior anticancer efficacy compared to equivalently-dosed individual or combination drugs via significant reduction in tumor burden over the course of treatment (p<0.01 by two-way ANOVA) (FIG. 6A). Using terminal deoxynucleotidyl transferase dUTP nick end labeling (TUNEL) we evaluated tumor-specific killing and organ toxicity, which confirmed DocRad-NP increased cell death in the residual tumor while avoiding other organs and systemic toxicities (FIGS. 6B and 6F). Using IHC on the residual tumor tissue, we tested pathway activation of Hsp90 via expression of phosphorylated the proline-rich Akt substrate of 40 kDa (PRAS40) and STAT3. IHC indicated that DocRad-NP sustained pathway inhibition up to four days beyond the last dosing, evidenced by reduced antibody staining at day 16 compared to the free drug combination (FIG. 6C). Blinded pathology assessment indicated over-expression of the NKG2D murine ligand receptor Murine ULBP-Like Transcript 1 (MULT-1) in the DocRad-NP cohort, while another receptor, retinoic acid early inducible gene 1 (Rae-1), remained of similar expression status across cohorts (FIG. 6D). Serial sections confirmed that regions of high MULT-1 expression tended to localize with increased incidence of CD49b⁺ cells, an indication of NK cells (39), while treatment conditions with regions of low expressing MULT-1 showed minimal infiltration (FIG. 6E).

### Example 1.7. NK Cells Affiliate with Drug-Induced Cell Death in Human Tumor Samples, Ex Vivo

Our data demonstrate an important dynamic relationship between NK cells and drug-induced death. We sought to confirm the important role of CD56+NK cells in response to multiple clinically-approved drugs as they affiliate with anticancer response or resistance in human tumors. To elucidate this, we deployed a human autologous ex vivo tumor model using primary human TNBC (Table 2). Fragments of living, fresh tumor biopsies and autologous patient-derived peripheral blood mononuclear cells (PBMC) were cultured on a substrate of tumor matrix proteins following a previously published procedure (25) (FIG. 7A). To this, we introduced clinically-approved (and off-label) anticancer drugs at their respective pharmacokinetic clinical max (Cₘₐₓ) (n=7, Table 3).

We developed a ‘sequential imaging’ strategy to study the TIME using 4 μm serial sections from formalin fixed paraffin embedded (FFPE) tissue following drug treatment, ex vivo, to locate: (1) regions of tumor vs. stroma (hematoxylin and eosin; H&E), (2) drug-induced apoptosis by cleaved caspase-3 and (3) NK cells via multiplex immunohistochemistry (mIHC; PanCK⁻CD3⁻CD56⁻), which demarcate a population poised for activity, excreting high amounts of immune-stimulating cytokines (40) (FIGS. 7B, 7G, 7H). This imaging approach allowed us to identify regions of tumor that were positive for apoptosis while also allowing us to pinpoint the location of NK cells within tumor vs. stroma (see methods, FIG. 7C).

We then performed several quantitative measurements: First, drug-induced cleaved caspase-3 in the tumor region determined as a log 2 fold change increase of 0.5 calculated between vehicle and drug (FIG. 7D). To quantify the role of NK cells with high drug-induced death (i.e. caspase-3 Hi) we deployed the HALO multiplex IHC software platform to quantify five independent parameters related to the location and density of NK cells within the tumor vs. stroma as well as the proximity to the tumor interface following drug pressure, which are features of TIL that contribute an anticancer effect (19). We then used Spearman correlation rank order analysis to compare these with drug effect. We made two key observations: 1) we identified a higher correlation within the five cellular metrics in the caspase-3 Hi vs. Lo cohorts, with a direct affiliation between NK cells within the tumor vs. stroma and proximity to tumor interface in relationship to drug-induced caspase-3 (FIG. 7E), and 2) a significant increase in NK cell density within the tumor (p<0.05) as well as a trend towards diminishing of the distance between NK cells and tumor in the caspase-3 Hi vs Lo cohort when comparing the vehicle treatment to drug treatment (FIG. 7F). These preliminary observations support a critical role for the dynamics of NK cells as they are linked to drug-induced cell death in human cancers.

TABLE 2
Patient demographic details and metadata for ex vivo experiments.

| | | | Anatomic | | | | |
|---|---|---|---|---|---|---|---|
| | Age, | | site of | Hormonal | Primary/ | | |
| Patient | years | Gender | specimen | Status | Metastasis | Grade | Drug treatments |
| IIOC-00a | 60 | Female | Liver | TNBC | Metastatic | Poorly | Vehicle, vinorelbine |
| | | | | | | differentiated | |
| | | | | | | carcinoma | |
| IIOC-00b | 52 | Female | Right | TNBC | Metastatic | Well | Vehicle, carbo + gem, |
| | | | anterior | | | diffrentiated | gemcitabine, doxil, |
| | | | chest wall | | | lobular | capecitabine, |
| | | | | | | carcinoma | pembrolizumab, eribulin, |
| | | | | | | | abraxane |
| IIOC-00c | 84 | Female | Liver | TNBC | Metastatic | Poorly | Vehicle, |
| | | | | | | diffrentiated | carbo + paclitaxel |
| | | | | | | carcinoma | |
| IIOC-00d | 37 | Female | Liver | TNBC | Metastatic | Poorly | Vehicle, carbo + gem, |
| | | | | | | diffrentiated | carboplatin, gemcitabine, |
| | | | | | | carcinoma | eribulin, |
| | | | | | | | prembrolizumab, |
| | | | | | | | capecitabine |
| IIOC-00e | 67 | Female | Left breast | TNBC | Metastatic | Poorly | Vehicle, pembrolizumab, |
| | | | | | | diffrentiated | anastrozole, |
| | | | | | | carcinoma | trastuzumab, |
| | | | | | | | gemcitabine, |
| | | | | | | | vinorelbine, eribulin, |
| | | | | | | | capecitabine |
| IIOC-00f | 60 | Female | Posterior | TNBC | Metastatic | Moderately | Vehicle, ixabipilone, |
| | | | right | | | diffrentiated | pembrolizumab, |
| | | | hepatic | | | carcinoma | docetaxel, vinorelbine, |
| | | | lobe | | | | doxil |
| IIOC-00g | 70 | Female | Liver | TNBC | Metastatic | Poorly | Vehicle, doxil, |
| | | | | | | differentiated | capecitabine, |
| | | | | | | carcinoma | gemcitabine citibine, |
| | | | | | | | eribulin, abraxane, |
| | | | | | | | paclitaxel, carboplatin, |
| | | | | | | | pembrolizumab |

TABLE 3
Drug concentrations for ex vivo experiments.

| Drug | Cmax (concentration used) | |
|---|---|---|
| Ixabepilone | 252 | ng/ml |
| Gemcitabine | 22.3 | μg/ml |
| Pembrolizumab | 65.7 | μg/ml |
| Vinorelbine | 126.6 | ng/ml |
| Anastrozole | 13.7 | mg/ml |
| Carboplatin | 37.1 | μg/ml |
| Paclitaxel | 6.91 | μM |
| Abraxane | 22 | μg/ml |
| Trastuzumab | 239 | μg/mL |
| Doxil | 0.93 | μg/ml |
| Docetaxel | 2 | μg/ml |
| Eribulin | 371 | ng/ml |
| Capecitabine | 3.9 | μg/ml |

### Example 2. Lipid-Targeted Nanotherapeutics Increase Killing of Drug-Induced Resistant Cancer Cells

Despite the risks associated with cytotoxic cancer chemotherapies, such as taxanes and anthracyclines, they remain a key part of treatment for more than half a century (64). A developing paradigm to improve the delivery of drugs directly to tumors and reduce toxicity to normal tissue and cells is the blending of engineering with biology (65). A variety of materials and technologies have been deployed to achieve this goal, which includes the use of lipids, polymers, inorganic carriers, hydrogels and even plasmonic strategies that exploit thermal dynamics (65). However, drug resistances have been shown to affect both the therapies themselves and the bioengineering strategies that are used to improve treatment response (67). Therefore, rational development of engineered nanotherapeutics that harness discoveries in cancer biology and drug resistance may overcome many of these challenges.

Nanotherapeutics for cancer often harness protein and nucleic acid biomarkers to target payloads (68). For example, decorating nanoparticles with aptamers, antibodies, proteins and small peptides, such as RGD, have shown improvement in reaching tumor cells and avoiding some of the toxicity associated with the cytotoxic payloads (66). However, resistance mechanisms such as endosomal recycling and molecular biological signals that rely on cell survival pathways can limit the efficacy of these approaches (67). For example, we recently made the discovery that dense lipid rafts are induced and accumulate on the surface of taxane-experienced cancer cells, which have phenotypically switched to a drug tolerant state (69). This phenotype allows cells to circumvent cytotoxic chemotherapy through a mechanism that involves binding of caveolin-1, scaffolding of Src Family Kinase (SFK)/hemopoietic cell kinase (Hck) and translocation of nuclear proteins that inhibit apoptosis (69). Separately, resistance to nanomedicines can manifest through extrinsic and physical barriers including endosomal recycling (70). This unique mechanism of resistance has been challenged using membrane fusion, osmotic pressure, nanoparticle swelling and membrane destabilization to bind and disrupt the endosomal packages (71). However, emerging approaches including plasmonics may provide novel opportunities to release drug payloads in a manner that potentially circumvents resistance via endosomal recycling (72). Harnessing these discoveries to improve the uptake of anticancer drugs into subpopulations of refractory cells, circumvent molecular and physical barriers to treatment response to enhance cell killing is a critical milestone in drug development.

Bioengineering-based cancer therapies that can improve anticancer activity in tumors and preferentially target mechanisms of resistance is a final frontier in the quest for durable clinical responses. In this study, we leveraged discoveries in cancer biology and cancer drug resistance to facilitate the design of two distinct nanotechnology-based therapeutic tools. In this example, we described how drug-induced resistance mechanisms can be exploited by engineered drug conjugates to deliver cell signaling disruptors that improve anticancer response.

Materials and Methods:

The following materials and methods were used in this Example.

Chemicals:

Cetyltrimethylammonium bromide (CTAB), Ethylene glycol (EG), Ammonia solution, Tetraethyl orthosilicate (TEOS), Ammonium nitrate (NH₄NO₃), Hydrazine (35 wt % in H2O), sodium azide, dimethyl sulfoxide (DMSO), Phosphate buffered saline (PBS), Hoechst 33342, paraformaldehyde, and Adriamycin were purchased from Sigma-Aldrich, MO, USA. 3-aminopropyltriethoxysilane (APS) was purchased from Gelest, PA, USA. Nanopure deionized (DI) water (18.1 MQ cm) was produced in house. 3-(4,5-Dimethylthiazol-2-yl)-2,5-Diphenyltetrazolium Bromide (MTT) was purchased from Thermo Fisher Scientific, MA, USA. MCF-7 cell line was obtained from ATCC, VA, USA. NBD-Ceramide (N-[12-[(7-nitro-2-1,3-benzoxadiazol-4-yl)amino]dodecanoyl]-D-erythro-sphingosine or C6 ceramide: (6-((N-(7-Nitrobenz-2-Oxa-1,3-Diazol-4-yl)amino)hexanoyl)Sphingosine)), NBD PC (1-oleoyl-2-{6-[(7-nitro-2-1,3-benzoxadiazol-4-yl)amino]hexanoyl}-sn-glycero-3-phosphocholine, 810132), NBD PA (1-oleoyl-2-{12-[(7-nitro-2-1,3-benzoxadiazol-4-yl)amino]dodecanoyl}-sn-glycero-3-phosphate (ammonium salt); 810176), NBD Cholesterol (25-[N-[(7-nitro-2-1,3-benzoxadiazol-4-yl)methyl]amino]-27-norcholesterol; 810250), NBD-PE (1,2-dimyristoyl-sn-glycero-3-phosphoethanolamine-N-(7-nitro-2-1,3-benzoxadiazol-4-yl) 810144), NBD-PG (1-oleoyl-2-{12-[(7-nitro-2-1,3-benzoxadiazol-4-yl)amino]dodecanoyl}-sn-glycero-3-[phospho-rac-(1-glycerol)](ammonium salt); 810166) and NBD-PGPE has been purchased from avanti polar lipids, USA. TopFluor PGPE: 1-palmitoyl-2-glutaroyl-sn-glycero-3-phosphoethanolamine-N-[4-(dipyrrometheneboron difluoride)butanoyl](ammonium salt).

Cell culture and generation of drug tolerant cancer cells in vitro:

MDA-MB-231 (ATCC) were cultured in DMEM containing 10% fetal bovine serum, MDA-MB-468 (ATCC), SUM-159 (ATCC), MCF-7 (ATCC), HeLa (ATCC) and 4T-1 mammary carcinoma cells (ATCC) were cultured in DMEM or RPMI containing 10% fetal bovine serum (Invitrogen, Carlsbad CA, USA) at 37° C. and 5% CO2. Generation of drug tolerance was performed as follows: cancer cells were plated at a density of 0.5×10⁵ cells ml⁻¹ and allowed to adhere for 24 hours onto cell culture plates. When the cells reached ˜75% confluency, they were treated with the cytotoxic drug, Docetaxel, at indicated concentrations for 48 hours and utilized for subsequent assays. Following washes with PBS, adherent cells were trypsinized and re-plated at a density of 2×10⁵ cells ml⁻¹ and cultured in serum-containing medium. After 24 hours incubation, the floating cells were removed and the remaining cells were washed with 1×PBS and considered to be the drug tolerant cancer cells (DTCCs). A population of drug naïve parental cancer cells (DNCCs) was always cultured alongside the DTCCs and fresh medium was added at the same interval that the DTCCs received fresh media.

Lipid Raft Imaging:

DNCCs or DTCCs were generated as described above and plated in eight chamber glass slides (BD Biosciences, San Jose, CA) at a concentration of 10⁵ cells/mL. Before lipid raft analysis, cells were first exposed to fluorescent lipids (NBD-PC or NBD-cholesterol; fluorescent in the green spectrum) at the indicated concentrations. Subsequently, lipid rafts were labelled by Vybrant™ Alexa Fluor™ 594 Lipid Raft Labeling Kit according to manufactures protocol. Briefly, cells were washed with PBS and CT-B (Cholera toxin subunit B) has been added with basal media (2 μg/ml) and incubated for 1 h at 4° C. Cells were washed 3 times with chilled PBS and Anti-CT-B (5 μL/mL in basal media) has been added to that. After incubation for 30 min at 4° C., the cells were fixed with 4% paraformaldehyde for 20 min. Cells were stained with DAPI and imaged by fluorescence or confocal microscopy. Fluorescent images were obtained using three channels on a NIKON Eclipse TI-U microscope with a 20× ELDW, 10 or 40× Plan-Apo objective lens (Nikon, Melville, NY). NIS Elements Viewer version 3.22 (Nikon, Melville, NY) software was used to capture the images to file.

Lipid Uptake by Flow Cytometry:

Cells were cultured as indicated, exposed to fluorescent lipids for the indicated amount of time and washed twice with PBS. Cells were then processed by flow cytometry to analyze fluorescent intensity of cells (Accuri C6, Bectin Dickinson Biosciences). Data analysis was performed using FlowJo software (Tree Star Inc., Ashland OR) and Accuri cFlow plus software to obtain and confirm mean fluorescent intensity and proportion of positively expressed cells. Vehicle control was used to subtract for background noise and determine lipid uptake as a proportion of positive fluorescent signal and fluorescent signal intensity for all cells analyzed.

MTT assay:

MCF-7 cells were seeded in a 96-well plate (0.32 cm² growth area) at a density of 10⁵ cells per well and cultured to test the cytotoxicity of A-NOA and iNOVS. We then added them into the medium, respectively, for 72 h in 5% CO₂ at 37° C. At the end of the incubation, MTT solution (0.1 mg/mL) was added and incubated for another 4 h. The medium was then replaced with DMSO (50%) per well, and the absorbance was monitored using a microplate reader (Bio-TekELx800) at the wavelength of 595 nm. The cytotoxicity was expressed as the percentage of cell viability compared to untreated control cells. The optical density (OD) of the sample was measured at 570 nm with a microplate reader. The cytotoxicity (=(A−B)/A×100, where A is the absorbance of the cells incubated with the culture medium and Bis the absorbance of the cells incubated with the nanoparticles or the free molecules).

In Vitro Cell Viability Analyses:

Cells were seeded in a 96-well plate at a density of 10⁵ cells per well and cultured in 5% CO₂ at 37° C. for 48 or 72 h to test the cytotoxicity of SK101, SK-TS-101. Drugs were added into the medium for indicated amount of time. At the end of the incubation, 25 μL (MTS solution; Promega) was added and incubated for another 4 h. The medium was then replaced with 100 μL of dimethyl sulfoxide (DMSO) per well, and the absorbance was monitored using a microplate reader (Bio-TekELx800) at the wavelength of 595 nm. The cytotoxicity was expressed as the percentage of cell viability compared to untreated control cells. The optical density (OD) of the sample was measured at 570 nm with a microplate reader.

Calculation of Drug sensitivity index for drug screening was achieved using MTS assay results as follows:

$$Drug sensitivity index ( D S I ) = - \{ \frac{\begin{matrix}Average viability for drug ‘ X ’ in \\ DNCCs ( 0.01 , 0.1 , 1. , 10 μM )\end{matrix}}{\begin{matrix}Average viability for drug ‘ X ’ in \\ DTCCs ( 0.01 , 0.1 , 1. ,\end{matrix}} \}$$

$$Higher D S I = more effective kill in DTCCs$$

In Vivo Studies:

Syngeneic mice model was generated using 4T1 breast cancer cells. Cells (1×10⁶) were implanted subcutaneously in the flanks of 5-week-old female BALB/c mice. Once the tumor size was 35 mm³, the mice were treated with vehicle or docetaxel (10 mg/Kg) twice on alternate days. Further, depending on the treatment groups, the mice were treated everyday with vehicle, SK-101(25 mg/Kg), or SKTS-101 conjugate drug (25 mg/Kg equivalent). The tumors were measured using a Vernier caliper, and tumor volume (Vt) was calculated using the formula, L×B2/2, where L is the longest, and W is the shortest dimension. Tissues were harvested for further studies and the weight of the harvested tumors from each of the mice groups were also measured. All animal procedures were approved by the Harvard Institutional Use and Care of Animals Committee.

### Synthesis of SK-101 and SKTS-101

### Synthetic Procedure and Characterization Data for SKTS-101

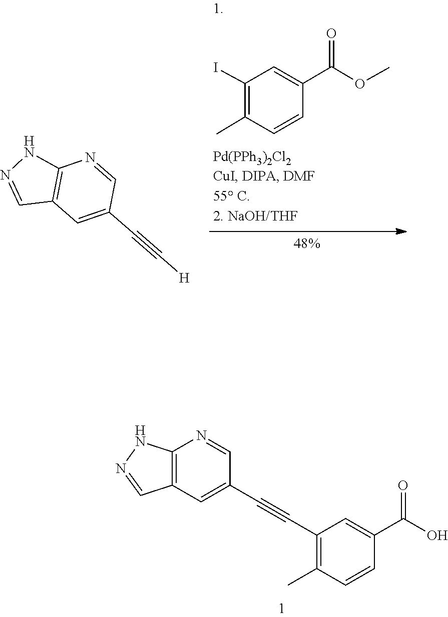 [SMILES: COC(=O)c1ccc(C)c(I)c1 ; C#Cc1cnc2[nH]ncc2c1 ; Cc1ccc(C(=O)O)cc1C#Cc1cnc2[nH]ncc2c1]

### Synthesis of 3-[(1H-pyrazolo[3,4-b]pyridin-5-yl)ethynyl]-4-methylbenzoic acid (2)

To a dry flask were added 5-ethynyl-1H-pyrazolo[3,4-b]pyridine (260 mg, 1.8 mmol), methyl 3-iodo-4-methylbenzoate (500 mg, 1.8 mmol), copper(I) iodide (35 mg, 0.18 mmol), and bis(triphenylphosphine)palladium(II) dichloride (63 mg, 0.09 mmol). The flask was evacuated and filled with nitrogen; and diisopropylamine (1 mL, 0.72 g, 7.2 mmol), and dry degassed DMF (10 mL) were added. Nitrogen was bubbled through the solution for 5 minutes. The reaction was then heated at 55° C. for 8 h. The reaction was subsequently taken up in EtOAc (100 mL) and the solution was filtered through celite, and washed with sat. NH₄Cl, sat. NaHCO₃, and brine (100 mL each). The organic layer was dried over Na₂SO₄, and filtered. The volatiles were evaporated, and the residue was suspended in Et₂O (50 mL) and stirred for 16 h. The suspension was filtered, and washed with additional Et₂O. The solid was collected and dissolved in THF (50 mL) and 1 M NaOH (50 mL), and stirred for 8 h. The solution was then cooled in an ice-bath and acidified by addition of 1 N HCl. The precipitate was filtered, collected, and washed with cold EtOAc, and Et₂O, and dried under vacuum. The product, 3-[(1H-pyrazolo[3,4-b]pyridin-5-yl)ethynyl]-4-methylbenzoic acid (2), was obtained without the need for further purification in 48% yield (240 mg, 0.86 mmol), over 2 steps.

¹H NMR (400 MHz, DMSO-d6): δ=2.55 (s, 3H), 7.46 (d, J=8.0 Hz, 1H), 7.84 (d, J=8.0 Hz, 1H), 8.05 (s, 1H), 8.20 (s, 1H), 8.50 (s, 1H), 8.71 (s, 1H).

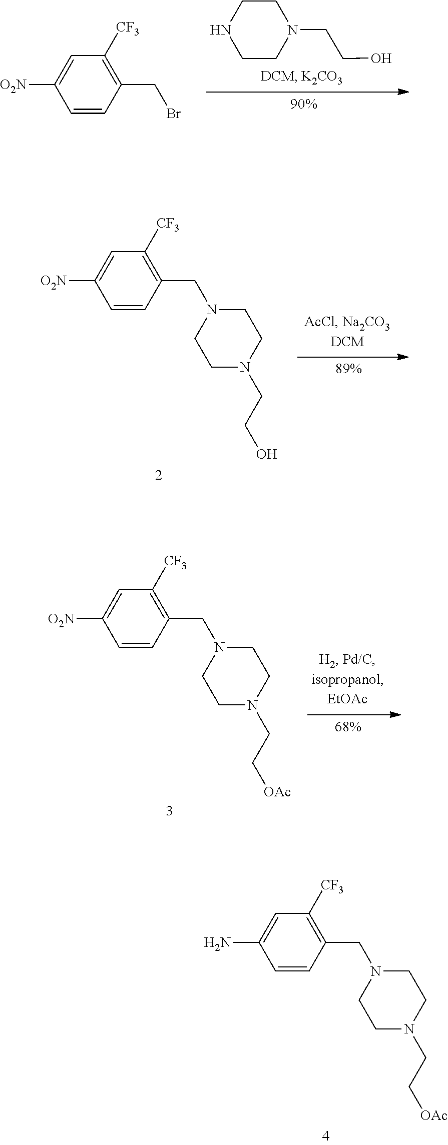 [SMILES: O=[N+]([O-])c1ccc(CBr)c(C(F)(F)F)c1 ; OCCN1CCNCC1 ; O=[N+]([O-])c1ccc(CN2CCN(CCO)CC2)c(C(F)(F)F)c1 ; CC(=O)OCCN1CCN(Cc2ccc([N+](=O)[O-])cc2C(F)(F)F)CC1 ; CC(=O)OCCN1CCN(Cc2ccc(N)cc2C(F)(F)F)CC1]

### Synthesis of 2-{4-[4-nitro-2-(trifluoromethyl)benzyl]piperazin-1-yl}ethan-1-ol (3)

To a dry flask were added 1-(bromomethyl)-4-nitro-2-(trifluoromethyl)benzene (500 mg, 1.8 mmol), K₂CO₃ (243 mg, 1.8 mmol), and dry DCM (10 mL). The solution was cooled in an ice-bath, and 2-(piperazin-1-yl)ethan-1-ol was added dropwise. (260 mg, 0.26 mL, 2.0 mmol). The reaction was allowed to stir for 8 h under nitrogen. The reaction was again cooled in ice, filtered, and volatiles were evaporated. The residue was purified by silica gel chromatography, using a gradient of DCM:MeOH:TEA (99:0:1 to 90:9:1). The product 3 was obtained in 90% yield (540 mg, 1.62 mmol).

### Synthesis of 2-{4-[4-nitro-2-(trifluoromethyl)benzyl]piperazin-1-yl}ethyl acetate (4)

To a dry flask were added 2-{4-[4-nitro-2-(trifluoromethyl)benzyl]piperazin-1-yl}ethan-1-ol (2) (540 mg, 1.62 mmol), and K₂CO₃ (243 mg, 1.8 mmol), and dry DCM (10 mL). The solution was cooled in an ice-bath, and acetyl chloride (127 mg, 0.11 mL, 1.62 mmol) was added dropwise to the solution. The reaction was allowed to stir for 8 h under nitrogen. The reaction was again cooled in ice, filtered, and washed with sat. NaHCO₃ (10 mL), and brine (10 mL). The organic layer was dried over Na₂SO₄, and filtered. The volatiles were evaporated. The residue was purified by silica gel chromatography, using a gradient of DCM:MeOH:TEA (99:0:1 to 90:9:1). The product 3 was obtained in 89% yield (525 mg, 1.4 mmol).

¹H NMR (400 MHz, CDCl₃): δ=2.04 (s, 3H), 2.51-2.55 (m, 8H), 2.63 (t, J=6.0 Hz, 2H), 3.72 (s, 2H), 4.18 (t, J=6.0 Hz, 2H), 8.08 (d, J=8.8 Hz, 1H), 8.35 (d, J=8.4 Hz, 1H), 8.47 (s, 1H).

### Synthesis of 2-{4-[4-amino-2-(trifluoromethyl)benzyl]piperazin-1-yl}ethyl acetate (4)

To a flask containing 2-{4-[4-nitro-2-(trifluoromethyl)benzyl]piperazin-1-yl}ethyl acetate (3) (525 mg, 1.4 mmol) were added degassed EtOAc (5 mL), degassed isopropanol (5 mL), and Pd/C (10% wt.) (250 mg). The flask was cooled, evacuated and the atmosphere replaced with H₂. The reaction was stirred at rt, and monitored for completion by LC/MS (about 6 h). The reaction was filtered through celite, and the volatiles were evaporated. The residue was further purified by silica gel chromatography, using a gradient of DCM:MeOH:TEA (99:0:1 to 90:9:1). The product 4 was obtained in 68% yield (330 mg, 0.95 mmol).

¹H NMR (400 MHz, CDCl₃): δ=2.04 (s, 3H), 2.44-2.53 (m, 8H), 2.62 (t, J=6.0 Hz, 2H), 3.51 (s, 2H), 3.76 (br s, 2H), 4.18 (t, J=6.0 Hz, 2H), 6.77 (d, J=8.4 Hz, 1H), 6.90 (s, 1H), 7.44 (d, J=8.4 Hz, 1H). LRMS: m/z calcd for C₁₆H₂₂F₃N₃O₂ [M+H]⁺: 346.2; found: 346.2.

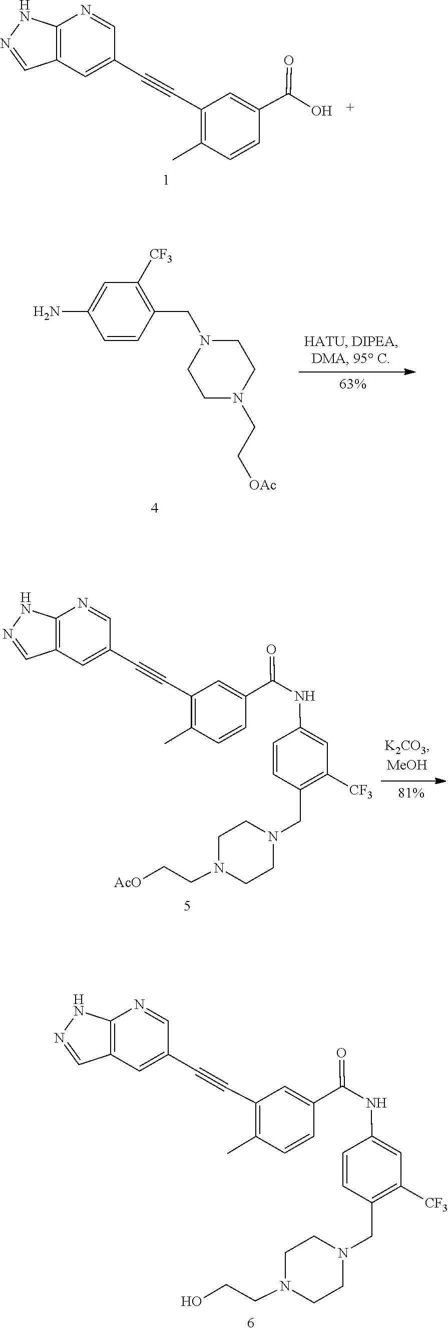 [SMILES: Cc1ccc(C(=O)O)cc1C#Cc1cnc2[nH]ncc2c1 ; CC(=O)OCCN1CCN(Cc2ccc(N)cc2C(F)(F)F)CC1 ; CC(=O)OCCN1CCN(Cc2ccc(NC(=O)c3ccc(C)c(C#Cc4cnc5[nH]ncc5c4)c3)cc2C(F)(F)F)CC1 ; Cc1ccc(C(=O)Nc2ccc(CN3CCN(CCO)CC3)c(C(F)(F)F)c2)cc1C#Cc1cnc2[nH]ncc2c1]

### Synthesis of 2-[4-(4-{3-[(1H-pyrazolo[3,4-b]pyridin-5-yl)ethynyl]-4-methylbenzamido}-2-(trifluoromethyl)benzyl)piperazin-1-yl]ethyl acetate (5)

To a dry flask were added carboxylic acid 2 (120 mg, 0.43 mmol), aniline 4 (150 mg, 0.43 mmol), and HATU (181 mg, 0.47 mmol). The flask was put under N₂ and dry DMA (3 mL) and dry DIPEA (122 mg, 0.17 mL, 0.95 mmol) were added. The reaction was heated at 95° C. for 8 h. The reaction was then taken up in EtOAc (50 mL) and washed with sat. NaHCO₃ (50 mL), and brine (2×50 mL). The organic layer was dried over Na₂SO₄, and filtered. The volatiles were evaporated. The residue was purified by silica gel chromatography, using a gradient of DCM:MeOH:TEA (99:0:1 to 90:9:1). The product 5 was obtained in 63% yield (165 mg, 0.27 mmol). LRMS: m/z calcd for C₃₂H₃₁F₃N₆O₃[M+H]⁺: 605.2; found: 605.2.

### Synthesis of 3-[(1H-pyrazolo[3,4-b]pyridin-5-yl)ethynyl]-N-(4-{[4-(2-hydroxyethyl)piperazin-1-yl]methyl}-3-(trifluoromethyl)phenyl)-4-methylbenzamide (6)

To a dry flask were added intermediate 5 (165 mg, 0.27 mmol), K₂CO₃ (15 mg, 0.1 mmol), and dry MeOH (3 mL). The reaction was stirred for 8 h at rt. The reaction was filtered, and the volatiles evaporated. The residue was purified by silica gel chromatography, using a gradient of DCM:MeOH:TEA (99:0:1 to 85:14:1). The product SK-101 was obtained in 81% yield (122 mg, 0.22 mmol).

LRMS: m/z calcd for C₃₀H₂₉F₃N₆O₂ [M+H]⁺: 563.2; found: 563.2.

1H NMR: (400 MHz, DMSO-d6): □ 10.55 (s, 1H), 8.74 (d, J=2.0 Hz, 1H), 8.53 (d, J=2.0 Hz, 1H), 8.26 \ddot{n} 8.18 (m, 3H), 8.07 (dd, J=8.5, 2.2 Hz, 1H), 7.93 (dd, J=8.0, 2.0 Hz, 1H), 7.71 (d, J=8.5 Hz, 1H), 7.53 (d, J=8.1 Hz, 1H), 4.35 (s, 1H), 3.56 (s, 2H), 3.48 (d, J=4.2 Hz, 2H), 2.59 (s, 3H), 2.38 (t, J=6.4 Hz, 9H).

13C NMR (100 MHz, DMSO-d6): □ 164.70, 151.00, 132.98, 132.11, 131.26, 130.57, 129.92, 123.52, 122.16, 113.99, 111.76, 91.90, 88.27, 79.19, 20.43.

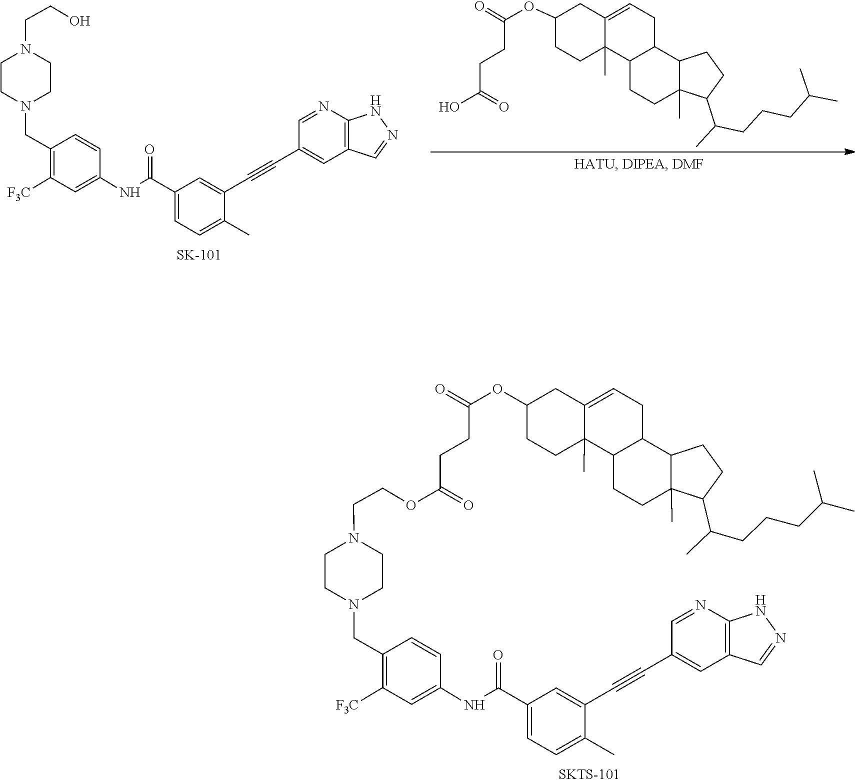 [SMILES: Cc1ccc(C(=O)Nc2ccc(CN3CCN(CCO)CC3)c(C(F)(F)F)c2)cc1C#Cc1cnc2[nH]ncc2c1 ; CC(C)CCCC(C)C1CCC2C3CC=C4CC(OC(=O)CCC(=O)O)CCC4(C)C3CCC12C ; Cc1ccc(C(=O)Nc2ccc(CN3CCN(CCOC(=O)CCC(=O)OC4CCC5(C)C(=CCC6C5CCC5(C)C(C(C)CCCC(C)C)CCC65)C4)CC3)c(C(F)(F)F)c2)cc1C#Cc1cnc2[nH]ncc2c1]

### Synthesis of 2-(4-(4-(3-((1H-pyrazolo[3,4-b]pyridin-5-yl)ethynyl)-4-methylbenzamido)-2-(trifluoromethyl)benzyl)piperazin-1-yl)ethyl (10,13-dimethyl-17-(6-methylheptan-2-yl)-2,3,4,7,8,9,10,11,12,13,14,15,16,17-tetradecahydro-1H-cyclopenta[a]phenanthren-3-yl) succinate

To a clean and dry flask were added compound 6 (30 mg, 0.053 mmol), DIPEA (0.212 mmol), HATU (0.1 mmol) and DMF. The reaction mix was stirred for 10 min at room temp under inert atmosphere. Cholesteryl hemisuccinate (0.08 mmol) and the mixture were stirred for 24 h. The reaction was monitored by TLC and the expected compound was purified by silica gel chromatography, using a gradient of DCM:MeOH: (100:0 to 90:10). The final product SKTS-101 was obtained in 40% yield (21.8 mg, 0.02 mmol).

1H NMR (400 MHz, Chloroform-d): δ 8.74 (d, J=1.9 Hz, 1H), 8.29 (d, J=1.9 Hz, 1H), 8.06-7.99 (m, 1H), 7.97 (s, 1H), 7.89 (d, J=9.5 Hz, 1H), 7.83-7.73 (m, 2H), 7.70 (dd, J=5.7, 3.3 Hz, 1H), 7.53 (dd, J=5.7, 3.3 Hz, 1H), 7.40 (d, J=8.1 Hz, 1H), 5.35 (s, 1H), 4.59 (s, 1H), 4.23 (d, J=28.6 Hz, 3H), 3.66 (s, 2H), 3.15 (s, 1H), 2.75 (s, 2H), 2.70-2.53 (m, 8H), 2.31 (d, J=7.3 Hz, 2H), 1.85 (d, J=12.1 Hz, 4H), 1.64-1.17 (m, 26H), 1.01 (s, 3H), 0.88 (m, 14H). ¹³C NMR (100 MHz, DMSO-D6): δ 209.53, 203.20, 196.33, 165.19, 155.83, 151.48, 145.32, 144.18, 142.94, 138.85, 133.46, 132.59, 131.74, 131.05, 130.40, 128.64, 128.64, 127.74, 124.00, 123.45, 122.64, 114.47, 112.23, 92.37, 88.74, 79.66, 57.45, 52.70, 49.03, 44.56, 37.12, 20.90.

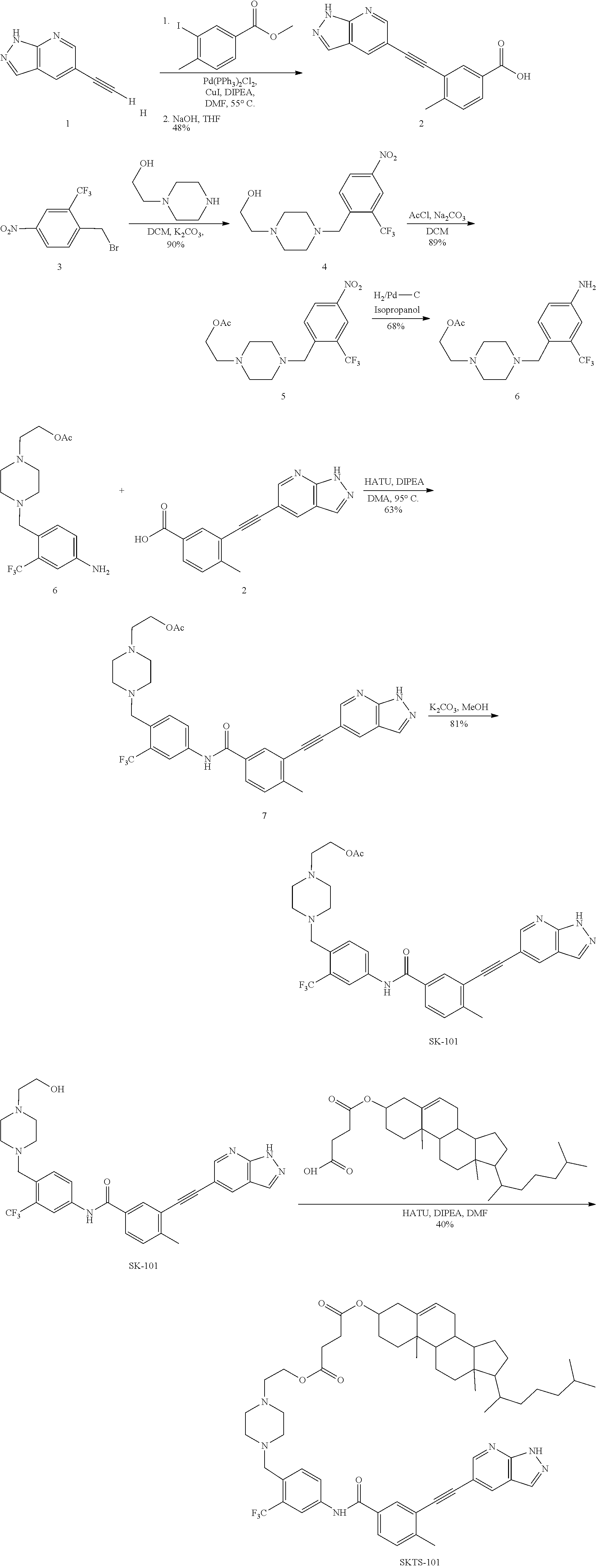

### Example 2.1. Screening Lipid Moieties that Preferentially Target Drug Tolerant Cancer Cells (DTCCs)

Cancer cells that have undergone acquired drug-induced resistance, or tolerance, can be collaterally sensitive to rationally-derived combination drug regimens (76, 77). To optimize for a combination regimen in drug tolerant cancer cells, we deployed an in-vitro model using the TNBC cell line, MDA-MB-231 (69). Briefly, cells were exposed to a high dose of docetaxel, a taxane chemotherapy routinely used in first-line TNBC (78), and selected cells based on their capacity to re-adhere after acute population outgrowth. The persisting cells are referred to hereafter as drug tolerant cancer cells (DTCCs) (FIG. 8A). We previously reported that DTCCs express a high concentration of plasma membrane lipid rafts compared to drug naïve cancer cells (DNCCs) (69). Indeed, we confirmed this phenomenon using epifluorescent imaging of lipid rafts via detecting lipid raft bound cholera toxin (FIG. 8B). Next, we developed a lipid-raft targeted screening protocol involving flow cytometry of fluorescently labeled lipids, which are characterized by different neutral or negative charges as well as unique log P values (FIG. 8H). Preferential binding and uptake into DTCCs was then evaluated (FIG. 8C). Based on this screen, we determined that phosphatidylcholine (PC) and cholesterol resulted in significantly increased uptake into DTCCs vs. DNCCs and, to a lesser degree, phosphatidic acid (PA) at levels higher than the other lipids tested (FIG. 8D). Indeed, we determined this effect was both dose and time dependent (FIGS. 8E and 8I). We focused on PC and cholesterol and assessed binding onto lipid rafts of DTCCs using fluorescent staining and co-localization experiments (FIG. 8F, light arrows). Finally, we determined that PC and cholesterol bound with the highest degree of specificity in multiple TNBC cell lines using epithelial-like MDA-MB-468 (FIG. 8G, black arrows). Based on this information we concluded that either PC or cholesterol could function as a moiety to selectively target the induction of lipid rafts that develop on DTCCs, and thus phosphatidyl choline or cholesterol can be used to improve drug target and drug uptake.

### REFERENCES

1. DeSantis C E, Ma J, Goding Sauer A, Newman L A, Jemal A. Breast cancer statistics, 2017, racial disparity in mortality by state. CA Cancer J Clin 2017; 67:439-48
2. Dent R, Trudeau M, Pritchard K I, Hanna W M, Kahn H K, Sawka C A, et al. Triple-negative breast cancer: clinical features and patterns of recurrence. Clin Cancer Res 2007; 13:4429-34
3. Collignon J, Lousberg L, Schroeder H, Jerusalem G. Triple-negative breast cancer: treatment challenges and solutions. Breast Cancer (Dove Med Press) 2016; 8:93-107
4. Pal S K, Childs B H, Pegram M. Triple negative breast cancer: unmet medical needs. Breast Cancer Res Treat 2011; 125:627-36
5. Yao H, He G, Yan S, Chen C, Song L, Rosol T J, et al. Triple-negative breast cancer: is there a treatment on the horizon? Oncotarget 2017; 8:1913-24
6. Villanueva M T. Cancer immunotherapy: Searching in the immune checkpoint black box. Nat Rev Drug Discov 2017; 16:599
7. Kwa M J, Adams S. Checkpoint inhibitors in triple-negative breast cancer (TNBC): Where to go from here. Cancer 2018; 124:2086-103
8. Katz H, Alsharedi M. Immunotherapy in triple-negative breast cancer. Med Oncol 2017; 35:13
9. Schmid P, Adams S, Rugo H S, Schneeweiss A, Barrios C H, Iwata H, et al. Atezolizumab and Nab-Paclitaxel in Advanced Triple-Negative Breast Cancer. N Engl J Med 2018; 379:2108-21
10. Redmond K M, Wilson T R, Johnston P G, Longley D B. Resistance mechanisms to cancer chemotherapy. Front Biosci 2008; 13:5138-54
11. Pisco A O, Huang S. Non-genetic cancer cell plasticity and therapy-induced sternness in tumour relapse: ‘What does not kill me strengthens me’. Br J Cancer 2015; 112:1725-32
12. Goldman A, Majumder B, Dhawan A, Ravi S, Goldman D, Kohandel M, et al. Temporally sequenced anticancer drugs overcome adaptive resistance by targeting a vulnerable chemotherapy-induced phenotypic transition. Nat Commun 2015; 6:6139
13. Zhao R, Davey M, Hsu Y C, Kaplanek P, Tong A, Parsons A B, et al. Navigating the chaperone network: an integrative map of physical and genetic interactions mediated by the hsp90 chaperone. Cell 2005; 120:715-27
14. Lu X, Xiao L, Wang L, Ruden D M. Hsp90 inhibitors and drug resistance in cancer: the potential benefits of combination therapies of Hsp90 inhibitors and other anti-cancer drugs. Biochem Pharmacol 2012; 83:995-1004
15. Fruman D A, Rommel C. PI3K and cancer: lessons, challenges and opportunities. Nat Rev Drug Discov 2014; 13:140-56
16. Wang H, Lu M, Yao M, Zhu W. Effects of treatment with an Hsp90 inhibitor in tumors based on 15 phase II clinical trials. Mol Clin Oncol 2016; 5:326-34
17. Binnewies M, Roberts E W, Kersten K, Chan V, Fearon D F, Merad M, et al. Understanding the tumor immune microenvironment (TIME) for effective therapy. Nat Med 2018; 24:541-50
18. Vivier E, Tomasello E, Baratin M, Walzer T, Ugolini S. Functions of natural killer cells. Nat Immunol 2008; 9:503-10
19. Yuan Y. Spatial Heterogeneity in the Tumor Microenvironment. Cold Spring Harb Perspect Med 2016; 6
20. Osipov A, Saung M T, Zheng L, Murphy A G. Small molecule immunomodulation: the tumor microenvironment and overcoming immune escape. J Immunother Cancer 2019; 7:224
21. Bradley C A. CD24—a novel ‘don't eat me’ signal. Nat Rev Cancer 2019; 19:541
22. Groh V, Steinle A, Bauer S, Spies T. Recognition of stress-induced MHC molecules by intestinal epithelial gammadelta T cells. Science 1998; 279:1737-40
23. Chou T C. Drug combination studies and their synergy quantification using the Chou-Talalay method. Cancer Res 2010; 70:440-6
24. Smalley M, Przedborski M, Thiyagarajan S, Pellowe M, Verma A, Brijwani N, et al. Integrating Systems Biology and an Ex vivo Human Tumor Model Elucidates PD-1 Blockade Response Dynamics. iScience 2020; 23:101229
25. Majumder B, Baraneedharan U, Thiyagarajan S, Radhakrishnan P, Narasimhan H, Dhandapani M, et al. Predicting clinical response to anticancer drugs using an ex vivo platform that captures tumour heterogeneity. Nat Commun 2015; 6:6169
26. Rangarao R, Smruti B K, Singh K, Gupta A, Batra S, Choudhary R K, et al. Practical consensus recommendations on management of triple-negative metastatic breast cancer. South Asian J Cancer 2018; 7:127-31
27. Arai K, Eguchi T, Rahman M M, Sakamoto R, Masuda N, Nakatsura T, et al. A Novel High-Throughput 3D Screening System for EMT Inhibitors: A Pilot Screening Discovered the EMT Inhibitory Activity of CDK2 Inhibitor SU9516. PLoS One 2016; 11:e0162394
28. Bauer S, Groh V, Wu J, Steinle A, Phillips J H, Lanier L L, et al. Activation of NK cells and T cells by NKG2D, a receptor for stress-inducible MICA. Science 1999; 285:727-9
29. Faisal M, Cumberland W, Champlin R, Fahey J L. Effect of recombinant human granulocyte-macrophage colony-stimulating factor administration on the lymphocyte subsets of patients with refractory aplastic anemia. Blood 1990; 76:1580-5
30. Schlahsa L, Jaimes Y, Blasczyk R, Figueiredo C. Granulocyte-colony-stimulatory factor: a strong inhibitor of natural killer cell function. Transfusion 2011; 51:293-305
31. Dayanc B E, Bansal S, Gure A O, Gollnick S O, Repasky E A. Enhanced sensitivity of colon tumour cells to natural killer cell cytotoxicity after mild thermal stress is regulated through HSF1-mediated expression of MICA. Int J Hyperthermia 2013; 29:480-90
32. Guettouche T, Boellmann F, Lane W S, Voellmy R. Analysis of phosphorylation of human heat shock factor 1 in cells experiencing a stress. BMC Biochem 2005; 6:4
33. Sharma S V, Agatsuma T, Nakano H. Targeting of the protein chaperone, HSP90, by the transformation suppressing agent, radicicol. Oncogene 1998; 16:2639-45
34. Lin T Y, Bear M, Du Z, Foley K P, Ying W, Barsoum J, et al. The novel HSP90 inhibitor STA-9090 exhibits activity against Kit-dependent and -independent malignant mast cell tumors. Exp Hematol 2008; 36:1266-77
35. Caldas-Lopes E, Cerchietti L, Ahn J H, Clement C C, Robles A I, Rodina A, et al. Hsp90 inhibitor PU—H71, a multimodal inhibitor of malignancy, induces complete responses in triple-negative breast cancer models. Proc Natl Acad Sci USA 2009; 106:8368-73
36. Lee M J, Ye A S, Gardino A K, Heijink A M, Sorger P K, MacBeath G, et al. Sequential application of anticancer drugs enhances cell death by rewiring apoptotic signaling networks. Cell 2012; 149:780-94
37. Davis M E, Chen Z G, Shin D M. Nanoparticle therapeutics: an emerging treatment modality for cancer. Nat Rev Drug Discov 2008; 7:771-82
38. Goldman A, Kulkarni A, Kohandel M, Pandey P, Rao P, Natarajan S K, et al. Rationally Designed 2-in-1 Nanoparticles Can Overcome Adaptive Resistance in Cancer. ACS Nano 2016; 10:5823-34
39. Arase H, Saito T, Phillips J H, Lanier L L. Cutting edge: the mouse NK cell-associated antigen recognized by DX5 monoclonal antibody is CD49b (alpha 2 integrin, very late antigen-2). J Immunol 2001; 167:1141-4
40. Poli A, Michel T, Theresine M, Andres E, Hentges F, Zimmer J. CD56 bright natural killer (NK) cells: an important NK cell subset. Immunology 2009; 126:458-65
41. Bae J, Munshi A, Li C, Samur M, Prabhala R, Mitsiades C, et al. Heat shock protein 90 is critical for regulation of phenotype and functional activity of human T lymphocytes and NK cells. J Immunol 2013; 190:1360-71
42. Maeda H, Nakamura H, Fang J. The EPR effect for macromolecular drug delivery to solid tumors: Improvement of tumor uptake, lowering of systemic toxicity, and distinct tumor imaging in vivo. Adv Drug Deliv Rev 2013; 65:71-9
43. Hu W, Wang G, Huang D, Sui M, Xu Y. Cancer Immunotherapy Based on Natural Killer Cells: Current Progress and New Opportunities. Front Immunol 2019; 10:1205
44. Souza-Fonseca-Guimaraes F, Cursons J, Huntington N D. The Emergence of Natural Killer Cells as a Major Target in Cancer Immunotherapy. Trends Immunol 2019; 40:142-58
45. Suck G, Odendahl M, Nowakowska P, Seidl C, Wels W S, Klingemann H G, et al. NK-92: an ‘off-the-shelf therapeutic’ for adoptive natural killer cell-based cancer immunotherapy. Cancer Immunol Immunother 2016; 65:485-92
46. Champsaur M, Lanier L L. Effect of NKG2D ligand expression on host immune responses. Immunol Rev 2010; 235:267-85
47. Gasser S, Orsulic S, Brown E J, Raulet D H. The DNA damage pathway regulates innate immune system ligands of the NKG2D receptor. Nature 2005; 436:1186-90
48. Cao, X., Tay, A., Guy, G. R., & Tan, Y. H. (1996). Activation and association of Stat3 with Src in v-Src-transformed cell lines. Molecular and Cellular Biology, 1595-1603.
49. Dou, F., Yuan, L.-D., & Zhu, J.-J. (2005). Heat Shock Protein 90 Indirectly Regulates ERK Activity by Affecting Raf Protein Metabolism. Acta biochimica et biophysica Sinica, 501-505.
50. Klinghoffer, R. A., Sachsenmaier, C., Cooper, J. A., & Soriano, P. (1999). Src family kinases are required for integrin but not PDGFR signal transduction. The EMBO Journal, 2459-2471.
51. Lamoureux, F., Thomas, C., Yin, M.-J., Kuruma, H., Beraldi, E., Fazli, L., . . . Gleave, M. E. (2011). Clusterin Inhibition Using OGX-011 Synergistically Enhances Hsp90 Inhibitor Activity by Suppressing the Heat Shock Response in Castrate-Resistant Prostate Cancer. Cancer research, 5838-5849.
52. Paulin, R., Meloche, J., Jacob, M. H., Bisserier, M., Courboulin, A., & Bonnet, S. (2011). Dehydroepiandrosterone inhibits the Src/STAT3 constitutive activation in pulmonary arterial hypertension. American Journal of Physiology—Heart and Circulatory Physiology, H1798-H1809.
53. Ram, P. T., & Iyengar, R. (2001). G protein coupled receptor signaling through the Src and Stat3 pathway: role in proliferation and transformation. Oncogene, 1601-1606.
54. Rodina, A., Vilenchik, M., Moulick, K., Aguirre, J., Kim, J., Chiang, A., . . . Chiosis, G. (2007). Selective compounds define Hsp90 as a major inhibitor of apoptosis in small-cell lung cancer. Nature Chemical Biology, 3, 498-507.
55. Rodina, A., Wang, T., Yan, P., Gomes, E. D., Dunphy, M. P., Pillarsetty, N., . . . Chiosis, G. (2016). The epichaperome is an integrated chaperome network that facilitates tumour survival. Nature, 397-401.
56. Singh, S., Trevino, J., Bora-Singhal, N., Coppola, D., Haura, E., Altiok, S., & Chellappan, S. P. (2012). EGFR/Src/Akt signaling modulates Sox2 expression and self-renewal of stem-like side-population cells in non-small cell lung cancer. Molecular Cancer, 73.
57. Sreedhar, A. S., Kalmar, E., Csermely, P., & Shen, Y.-F. (2004). Hsp90 isoforms: functions, expression and clinical importance. FEBS letters, 11-15.
58. Van Grol, J., Subauste, C., Andrade, R. M., Fujinaga, K., Nelson, J., & Subauste, C. S. (2010). HIV-1 Inhibits Autophagy in Bystander Macrophage/Monocytic Cells through Src-Akt and STAT3. PLoS one, e11733.
59. Workman, P., & Powers, M. V. (2007). Chaperoning cell death: a critical dual role for Hsp90 in small-cell lung cancer. Nature chemical biology, 455-457.
60. Wu, K., Chang, Q., Lu, Y., Qiu, P., Chen, B., Thakur, C., . . . Chen, F. (2013). Gefitinib resistance resulted from STAT3-mediated Akt. Oncotarget, 2430-2438.
61. Wu, Q., Wang, X., Wan, D., Li, J., & Yuan, Z. (2014). Crosstalk of JNK1-STAT3 is critical for RAW264.7 cell survival. Cellular Signalling, 2951-2960.
62. Xu, Y., & Lindquist, S. (1993). Heat-shock protein hsp90 governs the activity of pp60v-src kinase. Proceedings of the National Academy of Sciences of the United States of America, 7074-7078.
63. Yacoub, A., Han, S. I., Caron, R., Gilfor, D., Mooberry, S. L., Grant, S., & Dent, P. (2003). Sequence Dependent Exposure of Mammary Carcinoma Cells to Taxotere® and the MEK1/2 Inhibitor U0126 Causes Enhanced Cell Killing In Vitro. Cancer biology & therapy, 2(6), 670-676.
64. Schirrmacher, V., From chemotherapy to biological therapy: A review of novel concepts to reduce the side effects of systemic cancer treatment (Review). Int J Oncol, 2019. 54(2): p. 407-419.
65. Alexander-Bryant, A. A., W. S. Vanden Berg-Foels, and X. Wen, Bioengineering strategies for designing targeted cancer therapies. Adv Cancer Res, 2013. 118: p. 1-59.
66. Mitchell, M. J., et al., Engineering precision nanoparticles for drug delivery. Nat Rev Drug Discov, 2021. 20(2): p. 101-124.
67. Craig, M., et al., Engineering in Medicine To Address the Challenge of Cancer Drug Resistance: From Micro- and Nanotechnologies to Computational and Mathematical Modeling. Chem Rev, 2020.
68. Twomey, J. D., N. N. Brahme, and B. Zhang, Drug-biomarker co-development in oncology—20 years and counting. Drug Resist Updat, 2017. 30: p. 48-62.
69. Goldman, A., et al., Temporally sequenced anticancer drugs overcome adaptive resistance by targeting a vulnerable chemotherapy-induced phenotypic transition. Nat Commun, 2015. 6: p. 6139.
70. Slowing, I I, et al., Exocytosis of mesoporous silica nanoparticles from mammalian cells: from asymmetric cell-to-cell transfer to protein harvesting. Small, 2011. 7(11): p. 1526-32.
71. Smith, S. A., et al., The Endosomal Escape of Nanoparticles: Toward More Efficient Cellular Delivery. Bioconjug Chem, 2019. 30(2): p. 263-272.
72. Lee, S. E. and L. P. Lee, Biomolecular plasmonics for quantitative biology and nanomedicine. Curr Opin Biotechnol, 2010. 21(4): p. 489-97.
73. Wu, S. H., C. Y. Mou, and H. P. Lin, Synthesis of mesoporous silica nanoparticles. Chem Soc Rev, 2013. 42(9): p. 3862-75.
74. Robert I. Nooney, D. T., Yimei Chen, Robert Josephs, and Agnes E. Ostafin, Synthesis of Nanoscale Mesoporous Silica Spheres with Controlled Particle Size. Chemistry of Materials, 2002. 14(11): p. 4721-4728.
75. Lee, S. E., et al., Photonic gene circuits by optically addressable siRNA-Au nanoantennas. ACS Nano, 2012. 6(9): p. 7770-80.
76. Pluchino, K. M., et al., Collateral sensitivity as a strategy against cancer multidrug resistance. Drug Resist Updat, 2012. 15(1-2): p. 98-105.
77. Goldman, A., Tailoring combinatorial cancer therapies to target the origins of adaptive resistance. Molecular & Cellular Oncology, 2015. 3.
78. Pal, S. K., B. H. Childs, and M. Pegram, Triple negative breast cancer: unmet medical needs. Breast Cancer Res Treat, 2011. 125(3): p. 627-36.
79. Wang, Y., et al., GZD824 as a FLT3, FGFR1 and PDGFRalpha Inhibitor Against Leukemia In Vitro and In Vivo. Transl Oncol, 2020. 13(4): p. 100766.
80. Ye, W., et al., GZD824 suppresses the growth of human B cell precursor acute lymphoblastic leukemia cells by inhibiting the SRC kinase and PI3K/AKT pathways. Oncotarget, 2017. 8(50): p. 87002-87015.
81. Patel, A., et al., Novel roles of Src in cancer cell epithelial-to-mesenchymal transition, vascular permeability, microinvasion and metastasis. Life Sci, 2016. 157: p. 52-61.
82. Oh, N. and J. H. Park, Endocytosis and exocytosis of nanoparticles in mammalian cells. Int J Nanomedicine, 2014. 9 Suppl 1: p. 51-63.
83. Yanes, R. E., et al., Involvement of lysosomal exocytosis in the excretion of mesoporous silica nanoparticles and enhancement of the drug delivery effect by exocytosis inhibition. Small, 2013. 9(5): p. 697-704.
84. Fisher, B., et al., Doxorubicin-containing regimens for the treatment of stage II breast cancer: The National Surgical Adjuvant Breast and Bowel Project experience. J Clin Oncol, 1989. 7(5): p. 572-82.
85. Lipinski, C. A., et al., Experimental and computational approaches to estimate solubility and permeability in drug discovery and development settings. Adv Drug Deliv Rev, 2001. 46(1-3): p. 3-26.
86. Partlow, K. C., G. M. Lanza, and S. A. Wickline, Exploiting lipid raft transport with membrane targeted nanoparticles: a strategy for cytosolic drug delivery.

Biomaterials, 2008. 29(23): p. 3367-75.
87. Patra, J. K., et al., Nano based drug delivery systems: recent developments and future prospects. J Nanobiotechnology, 2018. 16(1): p. 71.
88. Vallet-Regi, M., et al., Mesoporous Silica Nanoparticles for Drug Delivery: Current Insights. Molecules, 2017. 23(1).
89. T, S. A., et al., Effect of dual stimuli responsive dextran/nanocellulose polyelectrolyte complexes for chemophotothermal synergistic cancer therapy. Int J Biol Macromol, 2019. 135: p. 776-789.
90. Park, J. H., et al., Gold Nanocage-Incorporated Poly(epsilon-Caprolactone) (PCL) Fibers for Chemophotothermal Synergistic Cancer Therapy. Pharmaceutics, 2019. 11(2).
91. Yin, T., et al., Multistimuli-responsive drug vehicles based on gold nanoflowers for chemophotothermal synergistic cancer therapy. Nanomedicine (Lond), 2018. 13(16): p. 1967-1983.
92. Xu, X., et al., Injectable, NIR/pH-Responsive Nanocomposite Hydrogel as Long-Acting Implant for Chemophotothermal Synergistic Cancer Therapy. ACS Appl Mater Interfaces, 2017. 9(24): p. 20361-20375.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A conjugate comprising a heat shock protein 90 (HSP90) inhibitor conjugated to a lipid.

2. The conjugate of claim 1, wherein the conjugate is an amphiphile.

3. The conjugate of claim 1, wherein the HSP90 inhibitor is radicicol or an analog thereof.

4. The conjugate of claim 3, wherein the HSP90 inhibitor is an analog of radicicol selected from KF25706, KF58333, radester, and pochonin D.

5. The conjugate of claim 1, wherein the lipid is a cholestanoid (preferably cholesterol), phosphatidylcholine (PC), phosphatidylethanolamine (PE), phosphatidic acid (PA), a phosphatidylserine (PS), or phosphatidylglycerol (PG).

6. The conjugate of claim 5, wherein the lipid is cholesterol or phosphatidylcholine (PC).

7. The conjugate of any of claims 1-2, wherein the HSP90 inhibitor is conjugated to the lipid via a linker.

8. The conjugate of claim 7, wherein the linker is selected from the group consisting of: —O—, —S—, —S—S—, —NR¹, —C(O)—, —C(O)O—, —C(O)NR¹, —SO—, —SO₂—, —SO₂NR¹—, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl; wherein one or more methylenes can be interrupted or terminated by O, S, S(O), SO₂, N(R¹)₂, C(O), C(O)O, C(O)NR¹, cleavable linking group, substituted or unsubstituted aryl, substituted or unsubstituted heteroaryl, substituted or unsubstituted heterocyclic, and wherein R¹ is hydrogen, acyl, aliphatic or substituted aliphatic, carbamate, or amide, pH-sensitive, glutathione sensitive, protease sensitive, peptide, disulfide, thioether, and β-glucuronide linkers.

9. The conjugate of claim 8, wherein the linker is C(O), C(O)CH₂CH₂C(O), or C(O)NH(CH₂)₂NHC(O)(CH₂)₂C(O).

10. The conjugate of claim 1, having the structure of Formula I or Formula II:

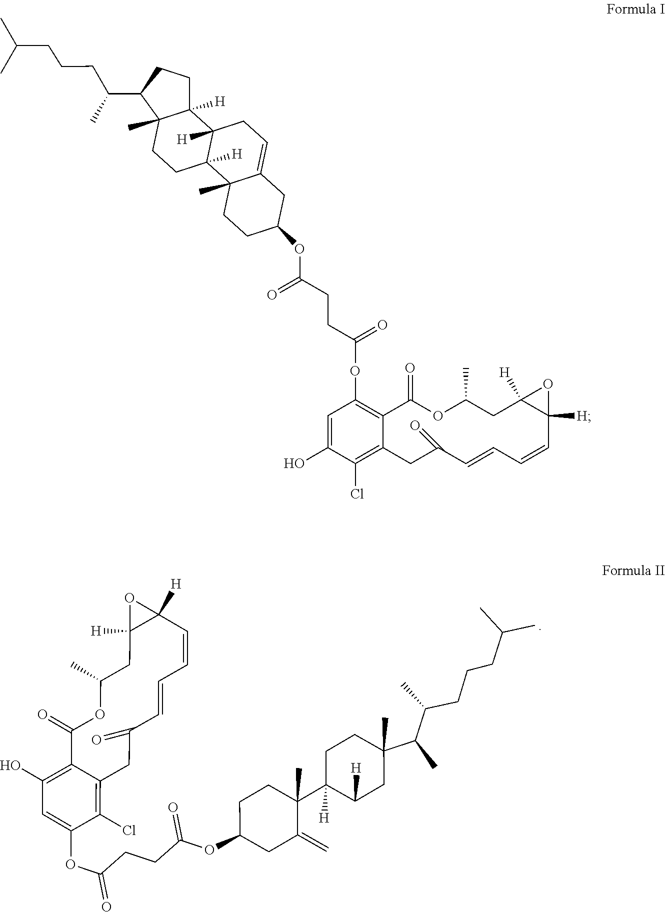

11. A composition comprising a conjugate of any of claims 1-10.

12. The composition of claim 11, wherein the composition comprises about 1% to about 99% (w/w) of the conjugate.

13. The composition claim 12, wherein the composition further comprises an additional lipid in addition to the conjugate.

14. The composition of claim 13, wherein the composition comprises about 1% to about 99% (w/w) of the additional lipid.

15. The composition of any of claims 11-14, wherein the additional lipid is a lipid conjugated with polyethylene glycol (PEG), optionally wherein the PEG conjugated lipid is selected from the group consisting of PEG conjugated diacylglycerols and dialkylglycerols, PEG-conjugated phosphatidylethanolamine and phosphatidic acid, PEG conjugated ceramides, PEG conjugated dialkylamines, PEG conjugated 1,2-diacyloxypropan-3-amines, and any combinations thereof.

16. The composition of claim 15, wherein the PEG conjugated lipid is 1,2-distearoyl-sn-glycem-3-phosphoethanolamine-N-[amino(polyethylene glycol)-2000] (DSPE-PEG2000).

17. The composition of any of claims 11-16, wherein the composition further comprises a phospholipid, preferably wherein the composition comprises about 1%> to about 99% (w/w) of the phospholipid.

18. The composition of claim 17, wherein the composition comprises the conjugate and the phospholipid in about 10:1 to about 1:10 ratio, and/or wherein the composition comprises the phospholipid and the lipid in about 10:1 to about 1:10 ratio.

19. The composition of claim 17, wherein the phospholipid is selected from phosphatidyl cholines, phosphatidyl cholines with acyl groups having 6 to 22 carbon atoms, phosphatidyl ethanolamines, phosphatidyl inositols, phosphatidic acids, phosphatidyl serines, sphingomyelin, phosphatidyl glycerols, and any combinations thereof, preferably wherein the phospholipid is selected from the group consisting of phosphatidylcholine, phosphatidylglycerol, lecithin, β,γ-dipalmitoyl-a-lecithin, sphingomyelin, phosphatidylserine, phosphatidic acid, N-(2,3-di(9-(Z)-octadecenyloxy))-prop-1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleyl-phosphatidylcholine, dimyristoyl phosphatidyl choline (DMPC), dioleoylphosphatidylethanolamine (DOPE), palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), -phosphatidylethanolamine (POPE), dioleoyl-phosphatidylethanolamine 4-(N-maleimidomethyl)-cyclohexane-1-carboxylate (DOPE-mal), and any combinations thereof.

20. The composition of claim 19, wherein the phosphatidylcholine is L-a-phosphatidylcholine.

21. The composition of any of claims 12-20, wherein the composition further comprises an anticancer agent in addition to the conjugate.

22. The composition of claim 21, wherein the anticancer agent is a taxane; a platinum compound, an alkylating agent; or an anti-metabolite.

23. The composition of claim 22, wherein the taxane is paclitaxel.

24. The composition of any of claims 12-23, wherein the composition comprises the conjugate, a PEG conjugated lipid, and a phospholipid.

25. The composition of claim 24, wherein the PEG conjugated lipid is DSPE-PEG2000 and the phospholipid is phosphatidylcholine.

26. The composition of claim 25, wherein the composition comprises the conjugate, the PEG conjugated lipid, and the phospholipid in ratio from about 10-0.1:10-0.1:10-0.1, or wherein the ratio is about 1.4:1:3 or about 10:5:1.

27. The composition of any of claim 12-26, wherein the composition is a nanoparticle, optionally a liposome or polymeric nanoparticle.

28. The composition of claim 27, wherein the nanoparticle is about 5 nm to about 500 nm in diameter, preferably wherein the nanoparticle 200-300 nm, or about 225 nm, in diameter.

29. A pharmaceutical composition comprising the conjugate or composition of any of claims 1-28, and a pharmaceutically acceptable carrier.

30. A method of treating cancer, the method comprising administering a therapeutically effective amount of the conjugate of any of claims 1-10 to a subject in need thereof.

31. The method of claim 30, further comprising administering an anticancer agent in addition to the conjugate.

32. The method of claim 31, wherein the anticancer agent is a taxane; a platinum compound, an alkylating agent; or an anti-metabolite, preferably wherein the taxane is paclitaxel, wherein the anticancer agent is administered before the conjugate of claims 1-10.

33. A method of treating cancer, the method comprising administering a therapeutically effective amount of the composition of any of claims 11-29 to a subject in need thereof.

34. The method of claims 30 to 33, wherein the cancer is selected from the group consisting of: breast cancer; ovarian cancer; glioma; gastrointestinal cancer; prostate cancer; carcinoma, lung carcinoma, hepatocellular carcinoma, testicular cancer; cervical cancer; endometrial cancer; bladder cancer; head and neck cancer; lung cancer; gastroesophageal cancer, and gynecological cancer, preferably wherein the cancer is triple negative breast cancer (TNBC).

35. The method of any of claims 30-34, further comprising administering one or more additional anti-cancer therapy to the patient.

36. The method of claim 32, wherein the additional therapy is selected from the group consisting of immunotherapy, preferably NK-cell based immunotherapy; surgery; chemotherapy, preferably a taxane; radiation therapy; thermotherapy; hormone therapy; laser therapy; anti-angiogenic therapy; and any combinations thereof; preferably wherein when the additional therapy is NK-cell based immunotherapy, the NK-cell based immunotherapy is administered after the composition of claims 11-29.

37. The conjugate of claims 1-10, the composition of any of claims 11-29, for use in a method of treating cancer in a subject in need thereof.

38. The conjugate or composition for the use of claim 37, wherein the cancer is selected from the group consisting of: breast cancer; ovarian cancer; glioma; gastrointestinal cancer; prostate cancer; carcinoma, lung carcinoma, hepatocellular carcinoma, testicular cancer; cervical cancer; endometrial cancer; bladder cancer; head and neck cancer; lung cancer; gastroesophageal cancer, and gynecological cancer, preferably wherein the cancer is triple negative breast cancer (TNBC).

39. The conjugate or composition for the use of claim 37 or 38, wherein the method further comprises co-administering one or more additional anti-cancer therapy to the patient.

40. The use of claim 39, wherein the additional therapy is selected from the group consisting of immunotherapy, preferably NK-cell based immunotherapy; surgery; chemotherapy; radiation therapy; thermotherapy; hormone therapy; laser therapy; anti-angiogenic therapy; and any combinations thereof.


SEQUENCE LISTING (3 sequences)

SEQ ID NO: 1 - protein, 9 aa, Artificial Sequence
  iRGD peptide
CRGDKGPDC

SEQ ID NO: 2 - protein, 7 aa, Artificial Sequence
  tCAR peptide
CARSKNK

SEQ ID NO: 3 - protein, 9 aa, Artificial Sequence
  Lyp-1 peptide
CGNKRTRGC